SEQUENCE LISTING (318 sequences)

SEQ ID NO: 1 - DNA, 148 bp, Saccharum spontaneum
  source 1..148: mol_type = genomic DNA
tagcccactaaaagaaagccttgcataacccttgatgtcactttattttggtttaagacagataagtctagctgagtaccttctcgtacttagggcgttgttcccattgttgttgtagatgattagatgtactacggctattgcgtca

SEQ ID NO: 2 - DNA, 169 bp, Saccharum spontaneum
  source 1..169: mol_type = genomic DNA
tagagcggagggcgttggagcgtctaggaatagatcgcgttctctccagtggcgagccgatctgagtgggaggacgtggatggggctcacgcggcgaggaggatgtggatgcggcgccgtcctggttcctgctcaaccacagcgacgctggcggctactgcacggtgga

SEQ ID NO: 3 - DNA, 237 bp, Saccharum spontaneum
  source 1..237: mol_type = genomic DNA
ctcgagcggtgtcggagaggaacagggggagctgccggatgatgacccggtcgtcggtggccccacccagctgacacgccaagcggaaatcggcgagccaaagttcgggtttggtctcgctgttgtacttggtgagagtcgtggggggcgaaaccgcgccgggagtatcatttcacgaatagccttactgaagacccgagggcccggaggctcgggtgaagggctacgatcctccga

SEQ ID NO: 4 - DNA, 142 bp, Saccharum spontaneum
  source 1..142: mol_type = genomic DNA
taccccaccaaaaagcctatcatgatccttggtgtcattttattttggttctgtcgggtaagtctagctgagtaccttctcgtactcagggctttgttcccacttgttgcagatggacagatgtagtatggttattgtatca

SEQ ID NO: 5 - DNA, 197 bp, Saccharum spontaneum
  source 1..197: mol_type = genomic DNA
gccaccggcctcgtggtcgtgagaccgaggagaacggtacctgggtatgggctggtagtcctcatccgtgctgtcagaagtcacatcctcaggaagatcagcaggctgagcgtcaagatcggcaagctctgcctctgcaagctcatcaactgtggcgtcttgctcagctgctgtctcaggaacctcaggtcgctcac

SEQ ID NO: 6 - DNA, 167 bp, Saccharum spontaneum
  source 1..167: mol_type = genomic DNA
gcggtgaataacgcgcaaagggtgagaaggctcacaccggatctacgggttacacagaggtacacatccatcgattctaacacgggcgtccggctcggcgccggggcgcggcgaggggtggagtggatagaactcgcagaagaagacgtgttactgtctgtccgcac

SEQ ID NO: 7 - DNA, 13 bp, synthetic construct
  misc_feature 1..13: note = Synthetic DNA
  source 1..13: mol_type = other DNA
taagagacagaga

SEQ ID NO: 8 - DNA, 13 bp, synthetic construct
  misc_feature 1..13: note = Synthetic DNA
  source 1..13: mol_type = other DNA
taagagacagagc

SEQ ID NO: 9 - DNA, 13 bp, synthetic construct
  misc_feature 1..13: note = Synthetic DNA
  source 1..13: mol_type = other DNA
taagagacagcgt

SEQ ID NO: 10 - DNA, 13 bp, synthetic construct
  misc_feature 1..13: note = Synthetic DNA
  source 1..13: mol_type = other DNA
taagagacagcta

SEQ ID NO: 11 - DNA, 13 bp, synthetic construct
  misc_feature 1..13: note = Synthetic DNA
  source 1..13: mol_type = other DNA
taagagacagctc

SEQ ID NO: 12 - DNA, 13 bp, synthetic construct
  misc_feature 1..13: note = Synthetic DNA
  source 1..13: mol_type = other DNA
taagagacaggct

SEQ ID NO: 13 - DNA, 13 bp, synthetic construct
  misc_feature 1..13: note = Synthetic DNA
  source 1..13: mol_type = other DNA
taagagacaggta

SEQ ID NO: 14 - DNA, 13 bp, synthetic construct
  misc_feature 1..13: note = Synthetic DNA
  source 1..13: mol_type = other DNA
taagagacaggtc

SEQ ID NO: 15 - DNA, 13 bp, synthetic construct
  misc_feature 1..13: note = Synthetic DNA
  source 1..13: mol_type = other DNA
taagagacagtac

SEQ ID NO: 16 - DNA, 13 bp, synthetic construct
  misc_feature 1..13: note = Synthetic DNA
  source 1..13: mol_type = other DNA
taagagacagtca

SEQ ID NO: 17 - DNA, 13 bp, synthetic construct
  misc_feature 1..13: note = Synthetic DNA
  source 1..13: mol_type = other DNA
taagagacagtga

SEQ ID NO: 18 - DNA, 13 bp, synthetic construct
  misc_feature 1..13: note = Synthetic DNA
  source 1..13: mol_type = other DNA
taagagacagttg

SEQ ID NO: 19 - DNA, 66 bp, synthetic construct
  misc_feature 1..66: note = Synthetic DNA
  source 1..66: mol_type = other DNA
caagcagaagacggcatacgagattcgtcagagtctcgtgggctcggagatgtgtataagagacag

SEQ ID NO: 20 - DNA, 66 bp, synthetic construct
  misc_feature 1..66: note = Synthetic DNA
  source 1..66: mol_type = other DNA
caagcagaagacggcatacgagatcgctagcggtctcgtgggctcggagatgtgtataagagacag

SEQ ID NO: 21 - DNA, 66 bp, synthetic construct
  misc_feature 1..66: note = Synthetic DNA
  source 1..66: mol_type = other DNA
caagcagaagacggcatacgagattctcagtagtctcgtgggctcggagatgtgtataagagacag

SEQ ID NO: 22 - DNA, 66 bp, synthetic construct
  misc_feature 1..66: note = Synthetic DNA
  source 1..66: mol_type = other DNA
caagcagaagacggcatacgagatcgtagatagtctcgtgggctcggagatgtgtataagagacag

SEQ ID NO: 23 - DNA, 66 bp, synthetic construct
  misc_feature 1..66: note = Synthetic DNA
  source 1..66: mol_type = other DNA
caagcagaagacggcatacgagatacgagcaggtctcgtgggctcggagatgtgtataagagacag

SEQ ID NO: 24 - DNA, 66 bp, synthetic construct
  misc_feature 1..66: note = Synthetic DNA
  source 1..66: mol_type = other DNA
caagcagaagacggcatacgagatatacgtgagtctcgtgggctcggagatgtgtataagagacag

SEQ ID NO: 25 - DNA, 66 bp, synthetic construct
  misc_feature 1..66: note = Synthetic DNA
  source 1..66: mol_type = other DNA
caagcagaagacggcatacgagatgtctagaggtctcgtgggctcggagatgtgtataagagacag

SEQ ID NO: 26 - DNA, 66 bp, synthetic construct
  misc_feature 1..66: note = Synthetic DNA
  source 1..66: mol_type = other DNA
caagcagaagacggcatacgagatagtcgacagtctcgtgggctcggagatgtgtataagagacag

SEQ ID NO: 27 - DNA, 66 bp, synthetic construct
  misc_feature 1..66: note = Synthetic DNA
  source 1..66: mol_type = other DNA
caagcagaagacggcatacgagatctcacagagtctcgtgggctcggagatgtgtataagagacag

SEQ ID NO: 28 - DNA, 66 bp, synthetic construct
  misc_feature 1..66: note = Synthetic DNA
  source 1..66: mol_type = other DNA
caagcagaagacggcatacgagatagacataggtctcgtgggctcggagatgtgtataagagacag

SEQ ID NO: 29 - DNA, 66 bp, synthetic construct
  misc_feature 1..66: note = Synthetic DNA
  source 1..66: mol_type = other DNA
caagcagaagacggcatacgagatagcgacgcgtctcgtgggctcggagatgtgtataagagacag

SEQ ID NO: 30 - DNA, 66 bp, synthetic construct
  misc_feature 1..66: note = Synthetic DNA
  source 1..66: mol_type = other DNA
caagcagaagacggcatacgagattgatagaggtctcgtgggctcggagatgtgtataagagacag

SEQ ID NO: 31 - DNA, 66 bp, synthetic construct
  misc_feature 1..66: note = Synthetic DNA
  source 1..66: mol_type = other DNA
caagcagaagacggcatacgagatgacgactggtctcgtgggctcggagatgtgtataagagacag

SEQ ID NO: 32 - DNA, 66 bp, synthetic construct
  misc_feature 1..66: note = Synthetic DNA
  source 1..66: mol_type = other DNA
caagcagaagacggcatacgagattgtgctcagtctcgtgggctcggagatgtgtataagagacag

SEQ ID NO: 33 - DNA, 66 bp, synthetic construct
  misc_feature 1..66: note = Synthetic DNA
  source 1..66: mol_type = other DNA
caagcagaagacggcatacgagatatgagctggtctcgtgggctcggagatgtgtataagagacag

SEQ ID NO: 34 - DNA, 66 bp, synthetic construct
  misc_feature 1..66: note = Synthetic DNA
  source 1..66: mol_type = other DNA
caagcagaagacggcatacgagattctctcacgtctcgtgggctcggagatgtgtataagagacag

SEQ ID NO: 35 - DNA, 66 bp, synthetic construct
  misc_feature 1..66: note = Synthetic DNA
  source 1..66: mol_type = other DNA
caagcagaagacggcatacgagatgcagatcagtctcgtgggctcggagatgtgtataagagacag

SEQ ID NO: 36 - DNA, 66 bp, synthetic construct
  misc_feature 1..66: note = Synthetic DNA
  source 1..66: mol_type = other DNA
caagcagaagacggcatacgagattctgcagagtctcgtgggctcggagatgtgtataagagacag

SEQ ID NO: 37 - DNA, 66 bp, synthetic construct
  misc_feature 1..66: note = Synthetic DNA
  source 1..66: mol_type = other DNA
caagcagaagacggcatacgagatacgtgagcgtctcgtgggctcggagatgtgtataagagacag

SEQ ID NO: 38 - DNA, 66 bp, synthetic construct
  misc_feature 1..66: note = Synthetic DNA
  source 1..66: mol_type = other DNA
caagcagaagacggcatacgagatcgcgtgaggtctcgtgggctcggagatgtgtataagagacag

SEQ ID NO: 39 - DNA, 66 bp, synthetic construct
  misc_feature 1..66: note = Synthetic DNA
  source 1..66: mol_type = other DNA
caagcagaagacggcatacgagatcatactacgtctcgtgggctcggagatgtgtataagagacag

SEQ ID NO: 40 - DNA, 66 bp, synthetic construct
  misc_feature 1..66: note = Synthetic DNA
  source 1..66: mol_type = other DNA
caagcagaagacggcatacgagattctacacagtctcgtgggctcggagatgtgtataagagacag

SEQ ID NO: 41 - DNA, 66 bp, synthetic construct
  misc_feature 1..66: note = Synthetic DNA
  source 1..66: mol_type = other DNA
caagcagaagacggcatacgagatgatagatcgtctcgtgggctcggagatgtgtataagagacag

SEQ ID NO: 42 - DNA, 66 bp, synthetic construct
  misc_feature 1..66: note = Synthetic DNA
  source 1..66: mol_type = other DNA
caagcagaagacggcatacgagatgagcgtacgtctcgtgggctcggagatgtgtataagagacag

SEQ ID NO: 43 - DNA, 66 bp, synthetic construct
  misc_feature 1..66: note = Synthetic DNA
  source 1..66: mol_type = other DNA
caagcagaagacggcatacgagatcagagacagtctcgtgggctcggagatgtgtataagagacag

SEQ ID NO: 44 - DNA, 66 bp, synthetic construct
  misc_feature 1..66: note = Synthetic DNA
  source 1..66: mol_type = other DNA
caagcagaagacggcatacgagatcatagatggtctcgtgggctcggagatgtgtataagagacag

SEQ ID NO: 45 - DNA, 66 bp, synthetic construct
  misc_feature 1..66: note = Synthetic DNA
  source 1..66: mol_type = other DNA
caagcagaagacggcatacgagatagatgctagtctcgtgggctcggagatgtgtataagagacag

SEQ ID NO: 46 - DNA, 66 bp, synthetic construct
  misc_feature 1..66: note = Synthetic DNA
  source 1..66: mol_type = other DNA
caagcagaagacggcatacgagatctcatctcgtctcgtgggctcggagatgtgtataagagacag

SEQ ID NO: 47 - DNA, 66 bp, synthetic construct
  misc_feature 1..66: note = Synthetic DNA
  source 1..66: mol_type = other DNA
caagcagaagacggcatacgagattatctatggtctcgtgggctcggagatgtgtataagagacag

SEQ ID NO: 48 - DNA, 66 bp, synthetic construct
  misc_feature 1..66: note = Synthetic DNA
  source 1..66: mol_type = other DNA
caagcagaagacggcatacgagatagagtatcgtctcgtgggctcggagatgtgtataagagacag

SEQ ID NO: 49 - DNA, 66 bp, synthetic construct
  misc_feature 1..66: note = Synthetic DNA
  source 1..66: mol_type = other DNA
caagcagaagacggcatacgagatgtgactaggtctcgtgggctcggagatgtgtataagagacag

SEQ ID NO: 50 - DNA, 66 bp, synthetic construct
  misc_feature 1..66: note = Synthetic DNA
  source 1..66: mol_type = other DNA
caagcagaagacggcatacgagatctatgctcgtctcgtgggctcggagatgtgtataagagacag

SEQ ID NO: 51 - DNA, 66 bp, synthetic construct
  misc_feature 1..66: note = Synthetic DNA
  source 1..66: mol_type = other DNA
caagcagaagacggcatacgagatctgactacgtctcgtgggctcggagatgtgtataagagacag

SEQ ID NO: 52 - DNA, 66 bp, synthetic construct
  misc_feature 1..66: note = Synthetic DNA
  source 1..66: mol_type = other DNA
caagcagaagacggcatacgagattatcagtagtctcgtgggctcggagatgtgtataagagacag

SEQ ID NO: 53 - DNA, 66 bp, synthetic construct
  misc_feature 1..66: note = Synthetic DNA
  source 1..66: mol_type = other DNA
caagcagaagacggcatacgagatgagtctgcgtctcgtgggctcggagatgtgtataagagacag

SEQ ID NO: 54 - DNA, 66 bp, synthetic construct
  misc_feature 1..66: note = Synthetic DNA
  source 1..66: mol_type = other DNA
caagcagaagacggcatacgagatcagtcgcagtctcgtgggctcggagatgtgtataagagacag

SEQ ID NO: 55 - DNA, 66 bp, synthetic construct
  misc_feature 1..66: note = Synthetic DNA
  source 1..66: mol_type = other DNA
caagcagaagacggcatacgagatgacatctagtctcgtgggctcggagatgtgtataagagacag

SEQ ID NO: 56 - DNA, 66 bp, synthetic construct
  misc_feature 1..66: note = Synthetic DNA
  source 1..66: mol_type = other DNA
caagcagaagacggcatacgagatctgtgacggtctcgtgggctcggagatgtgtataagagacag

SEQ ID NO: 57 - DNA, 66 bp, synthetic construct
  misc_feature 1..66: note = Synthetic DNA
  source 1..66: mol_type = other DNA
caagcagaagacggcatacgagataagaggcagtctcgtgggctcggagatgtgtataagagacag

SEQ ID NO: 58 - DNA, 66 bp, synthetic construct
  misc_feature 1..66: note = Synthetic DNA
  source 1..66: mol_type = other DNA
caagcagaagacggcatacgagatctagtacggtctcgtgggctcggagatgtgtataagagacag

SEQ ID NO: 59 - DNA, 66 bp, synthetic construct
  misc_feature 1..66: note = Synthetic DNA
  source 1..66: mol_type = other DNA
caagcagaagacggcatacgagataggagtccgtctcgtgggctcggagatgtgtataagagacag

SEQ ID NO: 60 - DNA, 66 bp, synthetic construct
  misc_feature 1..66: note = Synthetic DNA
  source 1..66: mol_type = other DNA
caagcagaagacggcatacgagatcatgcctagtctcgtgggctcggagatgtgtataagagacag

SEQ ID NO: 61 - DNA, 66 bp, synthetic construct
  misc_feature 1..66: note = Synthetic DNA
  source 1..66: mol_type = other DNA
caagcagaagacggcatacgagatgtagagaggtctcgtgggctcggagatgtgtataagagacag

SEQ ID NO: 62 - DNA, 66 bp, synthetic construct
  misc_feature 1..66: note = Synthetic DNA
  source 1..66: mol_type = other DNA
caagcagaagacggcatacgagatcctctctggtctcgtgggctcggagatgtgtataagagacag

SEQ ID NO: 63 - DNA, 66 bp, synthetic construct
  misc_feature 1..66: note = Synthetic DNA
  source 1..66: mol_type = other DNA
caagcagaagacggcatacgagatagcgtagcgtctcgtgggctcggagatgtgtataagagacag

SEQ ID NO: 64 - DNA, 66 bp, synthetic construct
  misc_feature 1..66: note = Synthetic DNA
  source 1..66: mol_type = other DNA
caagcagaagacggcatacgagattcctctacgtctcgtgggctcggagatgtgtataagagacag

SEQ ID NO: 65 - DNA, 66 bp, synthetic construct
  misc_feature 1..66: note = Synthetic DNA
  source 1..66: mol_type = other DNA
caagcagaagacggcatacgagatgacgtacagtctcgtgggctcggagatgtgtataagagacag

SEQ ID NO: 66 - DNA, 66 bp, synthetic construct
  misc_feature 1..66: note = Synthetic DNA
  source 1..66: mol_type = other DNA
caagcagaagacggcatacgagatgactgtaggtctcgtgggctcggagatgtgtataagagacag

SEQ ID NO: 67 - DNA, 66 bp, synthetic construct
  misc_feature 1..66: note = Synthetic DNA
  source 1..66: mol_type = other DNA
caagcagaagacggcatacgagattcagtacagtctcgtgggctcggagatgtgtataagagacag

SEQ ID NO: 68 - DNA, 66 bp, synthetic construct
  misc_feature 1..66: note = Synthetic DNA
  source 1..66: mol_type = other DNA
caagcagaagacggcatacgagatcatgataggtctcgtgggctcggagatgtgtataagagacag

SEQ ID NO: 69 - DNA, 66 bp, synthetic construct
  misc_feature 1..66: note = Synthetic DNA
  source 1..66: mol_type = other DNA
caagcagaagacggcatacgagatgcatctcggtctcgtgggctcggagatgtgtataagagacag

SEQ ID NO: 70 - DNA, 66 bp, synthetic construct
  misc_feature 1..66: note = Synthetic DNA
  source 1..66: mol_type = other DNA
caagcagaagacggcatacgagattgcacagagtctcgtgggctcggagatgtgtataagagacag

SEQ ID NO: 71 - DNA, 66 bp, synthetic construct
  misc_feature 1..66: note = Synthetic DNA
  source 1..66: mol_type = other DNA
caagcagaagacggcatacgagatgagctatagtctcgtgggctcggagatgtgtataagagacag

SEQ ID NO: 72 - DNA, 66 bp, synthetic construct
  misc_feature 1..66: note = Synthetic DNA
  source 1..66: mol_type = other DNA
caagcagaagacggcatacgagatagtctgcagtctcgtgggctcggagatgtgtataagagacag

SEQ ID NO: 73 - DNA, 66 bp, synthetic construct
  misc_feature 1..66: note = Synthetic DNA
  source 1..66: mol_type = other DNA
caagcagaagacggcatacgagatcgctgtgagtctcgtgggctcggagatgtgtataagagacag

SEQ ID NO: 74 - DNA, 66 bp, synthetic construct
  misc_feature 1..66: note = Synthetic DNA
  source 1..66: mol_type = other DNA
caagcagaagacggcatacgagatctgatgtggtctcgtgggctcggagatgtgtataagagacag

SEQ ID NO: 75 - DNA, 66 bp, synthetic construct
  misc_feature 1..66: note = Synthetic DNA
  source 1..66: mol_type = other DNA
caagcagaagacggcatacgagatgcactctagtctcgtgggctcggagatgtgtataagagacag

SEQ ID NO: 76 - DNA, 66 bp, synthetic construct
  misc_feature 1..66: note = Synthetic DNA
  source 1..66: mol_type = other DNA
caagcagaagacggcatacgagattctgctcagtctcgtgggctcggagatgtgtataagagacag

SEQ ID NO: 77 - DNA, 66 bp, synthetic construct
  misc_feature 1..66: note = Synthetic DNA
  source 1..66: mol_type = other DNA
caagcagaagacggcatacgagattgtatctagtctcgtgggctcggagatgtgtataagagacag

SEQ ID NO: 78 - DNA, 66 bp, synthetic construct
  misc_feature 1..66: note = Synthetic DNA
  source 1..66: mol_type = other DNA
caagcagaagacggcatacgagatacagtgaggtctcgtgggctcggagatgtgtataagagacag

SEQ ID NO: 79 - DNA, 66 bp, synthetic construct
  misc_feature 1..66: note = Synthetic DNA
  source 1..66: mol_type = other DNA
caagcagaagacggcatacgagatatgcgatagtctcgtgggctcggagatgtgtataagagacag

SEQ ID NO: 80 - DNA, 66 bp, synthetic construct
  misc_feature 1..66: note = Synthetic DNA
  source 1..66: mol_type = other DNA
caagcagaagacggcatacgagatgagacatggtctcgtgggctcggagatgtgtataagagacag

SEQ ID NO: 81 - DNA, 66 bp, synthetic construct
  misc_feature 1..66: note = Synthetic DNA
  source 1..66: mol_type = other DNA
caagcagaagacggcatacgagatgtcatgtggtctcgtgggctcggagatgtgtataagagacag

SEQ ID NO: 82 - DNA, 66 bp, synthetic construct
  misc_feature 1..66: note = Synthetic DNA
  source 1..66: mol_type = other DNA
caagcagaagacggcatacgagattcatgatagtctcgtgggctcggagatgtgtataagagacag

SEQ ID NO: 83 - DNA, 66 bp, synthetic construct
  misc_feature 1..66: note = Synthetic DNA
  source 1..66: mol_type = other DNA
caagcagaagacggcatacgagatgtcatctcgtctcgtgggctcggagatgtgtataagagacag

SEQ ID NO: 84 - DNA, 66 bp, synthetic construct
  misc_feature 1..66: note = Synthetic DNA
  source 1..66: mol_type = other DNA
caagcagaagacggcatacgagattatctctagtctcgtgggctcggagatgtgtataagagacag

SEQ ID NO: 85 - DNA, 66 bp, synthetic construct
  misc_feature 1..66: note = Synthetic DNA
  source 1..66: mol_type = other DNA
caagcagaagacggcatacgagatctgatatagtctcgtgggctcggagatgtgtataagagacag

SEQ ID NO: 86 - DNA, 66 bp, synthetic construct
  misc_feature 1..66: note = Synthetic DNA
  source 1..66: mol_type = other DNA
caagcagaagacggcatacgagattacgcatggtctcgtgggctcggagatgtgtataagagacag

SEQ ID NO: 87 - DNA, 66 bp, synthetic construct
  misc_feature 1..66: note = Synthetic DNA
  source 1..66: mol_type = other DNA
caagcagaagacggcatacgagatcgtgagtggtctcgtgggctcggagatgtgtataagagacag

SEQ ID NO: 88 - DNA, 66 bp, synthetic construct
  misc_feature 1..66: note = Synthetic DNA
  source 1..66: mol_type = other DNA
caagcagaagacggcatacgagatgacacatggtctcgtgggctcggagatgtgtataagagacag

SEQ ID NO: 89 - DNA, 66 bp, synthetic construct
  misc_feature 1..66: note = Synthetic DNA
  source 1..66: mol_type = other DNA
caagcagaagacggcatacgagatacatgacggtctcgtgggctcggagatgtgtataagagacag

SEQ ID NO: 90 - DNA, 66 bp, synthetic construct
  misc_feature 1..66: note = Synthetic DNA
  source 1..66: mol_type = other DNA
caagcagaagacggcatacgagatgcgtctaggtctcgtgggctcggagatgtgtataagagacag

SEQ ID NO: 91 - DNA, 66 bp, synthetic construct
  misc_feature 1..66: note = Synthetic DNA
  source 1..66: mol_type = other DNA
caagcagaagacggcatacgagattcacgctggtctcgtgggctcggagatgtgtataagagacag

SEQ ID NO: 92 - DNA, 66 bp, synthetic construct
  misc_feature 1..66: note = Synthetic DNA
  source 1..66: mol_type = other DNA
caagcagaagacggcatacgagattcatgtacgtctcgtgggctcggagatgtgtataagagacag

SEQ ID NO: 93 - DNA, 66 bp, synthetic construct
  misc_feature 1..66: note = Synthetic DNA
  source 1..66: mol_type = other DNA
caagcagaagacggcatacgagattagtgacggtctcgtgggctcggagatgtgtataagagacag

SEQ ID NO: 94 - DNA, 66 bp, synthetic construct
  misc_feature 1..66: note = Synthetic DNA
  source 1..66: mol_type = other DNA
caagcagaagacggcatacgagatcacgataggtctcgtgggctcggagatgtgtataagagacag

SEQ ID NO: 95 - DNA, 66 bp, synthetic construct
  misc_feature 1..66: note = Synthetic DNA
  source 1..66: mol_type = other DNA
caagcagaagacggcatacgagatacacactggtctcgtgggctcggagatgtgtataagagacag

SEQ ID NO: 96 - DNA, 66 bp, synthetic construct
  misc_feature 1..66: note = Synthetic DNA
  source 1..66: mol_type = other DNA
caagcagaagacggcatacgagatagcatcacgtctcgtgggctcggagatgtgtataagagacag

SEQ ID NO: 97 - DNA, 66 bp, synthetic construct
  misc_feature 1..66: note = Synthetic DNA
  source 1..66: mol_type = other DNA
caagcagaagacggcatacgagattagtcgtagtctcgtgggctcggagatgtgtataagagacag

SEQ ID NO: 98 - DNA, 66 bp, synthetic construct
  misc_feature 1..66: note = Synthetic DNA
  source 1..66: mol_type = other DNA
caagcagaagacggcatacgagatgcatcgcggtctcgtgggctcggagatgtgtataagagacag

SEQ ID NO: 99 - DNA, 66 bp, synthetic construct
  misc_feature 1..66: note = Synthetic DNA
  source 1..66: mol_type = other DNA
caagcagaagacggcatacgagatatcatgtcgtctcgtgggctcggagatgtgtataagagacag

SEQ ID NO: 100 - DNA, 66 bp, synthetic construct
  misc_feature 1..66: note = Synthetic DNA
  source 1..66: mol_type = other DNA
caagcagaagacggcatacgagatgtactctagtctcgtgggctcggagatgtgtataagagacag

SEQ ID NO: 101 - DNA, 66 bp, synthetic construct
  misc_feature 1..66: note = Synthetic DNA
  source 1..66: mol_type = other DNA
caagcagaagacggcatacgagatagtgcatagtctcgtgggctcggagatgtgtataagagacag

SEQ ID NO: 102 - DNA, 66 bp, synthetic construct
  misc_feature 1..66: note = Synthetic DNA
  source 1..66: mol_type = other DNA
caagcagaagacggcatacgagatcgcatcaggtctcgtgggctcggagatgtgtataagagacag

SEQ ID NO: 103 - DNA, 66 bp, synthetic construct
  misc_feature 1..66: note = Synthetic DNA
  source 1..66: mol_type = other DNA
caagcagaagacggcatacgagatcgctataggtctcgtgggctcggagatgtgtataagagacag

SEQ ID NO: 104 - DNA, 66 bp, synthetic construct
  misc_feature 1..66: note = Synthetic DNA
  source 1..66: mol_type = other DNA
caagcagaagacggcatacgagattagctctagtctcgtgggctcggagatgtgtataagagacag

SEQ ID NO: 105 - DNA, 66 bp, synthetic construct
  misc_feature 1..66: note = Synthetic DNA
  source 1..66: mol_type = other DNA
caagcagaagacggcatacgagatgtcgataggtctcgtgggctcggagatgtgtataagagacag

SEQ ID NO: 106 - DNA, 66 bp, synthetic construct
  misc_feature 1..66: note = Synthetic DNA
  source 1..66: mol_type = other DNA
caagcagaagacggcatacgagatagctcgtagtctcgtgggctcggagatgtgtataagagacag

SEQ ID NO: 107 - DNA, 66 bp, synthetic construct
  misc_feature 1..66: note = Synthetic DNA
  source 1..66: mol_type = other DNA
caagcagaagacggcatacgagatacacagtagtctcgtgggctcggagatgtgtataagagacag

SEQ ID NO: 108 - DNA, 66 bp, synthetic construct
  misc_feature 1..66: note = Synthetic DNA
  source 1..66: mol_type = other DNA
caagcagaagacggcatacgagatcagatgtagtctcgtgggctcggagatgtgtataagagacag

SEQ ID NO: 109 - DNA, 66 bp, synthetic construct
  misc_feature 1..66: note = Synthetic DNA
  source 1..66: mol_type = other DNA
caagcagaagacggcatacgagatctctacaggtctcgtgggctcggagatgtgtataagagacag

SEQ ID NO: 110 - DNA, 66 bp, synthetic construct
  misc_feature 1..66: note = Synthetic DNA
  source 1..66: mol_type = other DNA
caagcagaagacggcatacgagatgtcactaggtctcgtgggctcggagatgtgtataagagacag

SEQ ID NO: 111 - DNA, 66 bp, synthetic construct
  misc_feature 1..66: note = Synthetic DNA
  source 1..66: mol_type = other DNA
caagcagaagacggcatacgagattgtactaggtctcgtgggctcggagatgtgtataagagacag

SEQ ID NO: 112 - DNA, 66 bp, synthetic construct
  misc_feature 1..66: note = Synthetic DNA
  source 1..66: mol_type = other DNA
caagcagaagacggcatacgagatacgctatggtctcgtgggctcggagatgtgtataagagacag

SEQ ID NO: 113 - DNA, 66 bp, synthetic construct
  misc_feature 1..66: note = Synthetic DNA
  source 1..66: mol_type = other DNA
caagcagaagacggcatacgagatatgtataggtctcgtgggctcggagatgtgtataagagacag

SEQ ID NO: 114 - DNA, 66 bp, synthetic construct
  misc_feature 1..66: note = Synthetic DNA
  source 1..66: mol_type = other DNA
caagcagaagacggcatacgagattgtgacaggtctcgtgggctcggagatgtgtataagagacag

SEQ ID NO: 115 - DNA, 66 bp, synthetic construct
  misc_feature 1..66: note = Synthetic DNA
  source 1..66: mol_type = other DNA
caagcagaagacggcatacgagatgacgtcaggtctcgtgggctcggagatgtgtataagagacag

SEQ ID NO: 116 - DNA, 66 bp, synthetic construct
  misc_feature 1..66: note = Synthetic DNA
  source 1..66: mol_type = other DNA
caagcagaagacggcatacgagatagatcgaggtctcgtgggctcggagatgtgtataagagacag

SEQ ID NO: 117 - DNA, 66 bp, synthetic construct
  misc_feature 1..66: note = Synthetic DNA
  source 1..66: mol_type = other DNA
caagcagaagacggcatacgagatatagtagagtctcgtgggctcggagatgtgtataagagacag

SEQ ID NO: 118 - DNA, 66 bp, synthetic construct
  misc_feature 1..66: note = Synthetic DNA
  source 1..66: mol_type = other DNA
caagcagaagacggcatacgagattatgactagtctcgtgggctcggagatgtgtataagagacag

SEQ ID NO: 119 - DNA, 66 bp, synthetic construct
  misc_feature 1..66: note = Synthetic DNA
  source 1..66: mol_type = other DNA
caagcagaagacggcatacgagattagagatggtctcgtgggctcggagatgtgtataagagacag

SEQ ID NO: 120 - DNA, 66 bp, synthetic construct
  misc_feature 1..66: note = Synthetic DNA
  source 1..66: mol_type = other DNA
caagcagaagacggcatacgagatagctgagagtctcgtgggctcggagatgtgtataagagacag

SEQ ID NO: 121 - DNA, 66 bp, synthetic construct
  misc_feature 1..66: note = Synthetic DNA
  source 1..66: mol_type = other DNA
caagcagaagacggcatacgagatacatctgagtctcgtgggctcggagatgtgtataagagacag

SEQ ID NO: 122 - DNA, 66 bp, synthetic construct
  misc_feature 1..66: note = Synthetic DNA
  source 1..66: mol_type = other DNA
caagcagaagacggcatacgagatgcgtgctagtctcgtgggctcggagatgtgtataagagacag

SEQ ID NO: 123 - DNA, 66 bp, synthetic construct
  misc_feature 1..66: note = Synthetic DNA
  source 1..66: mol_type = other DNA
caagcagaagacggcatacgagatacatgtaggtctcgtgggctcggagatgtgtataagagacag

SEQ ID NO: 124 - DNA, 66 bp, synthetic construct
  misc_feature 1..66: note = Synthetic DNA
  source 1..66: mol_type = other DNA
caagcagaagacggcatacgagatatagagaggtctcgtgggctcggagatgtgtataagagacag

SEQ ID NO: 125 - DNA, 66 bp, synthetic construct
  misc_feature 1..66: note = Synthetic DNA
  source 1..66: mol_type = other DNA
caagcagaagacggcatacgagatgtatctaggtctcgtgggctcggagatgtgtataagagacag

SEQ ID NO: 126 - DNA, 66 bp, synthetic construct
  misc_feature 1..66: note = Synthetic DNA
  source 1..66: mol_type = other DNA
caagcagaagacggcatacgagatatactgtagtctcgtgggctcggagatgtgtataagagacag

SEQ ID NO: 127 - DNA, 66 bp, synthetic construct
  misc_feature 1..66: note = Synthetic DNA
  source 1..66: mol_type = other DNA
caagcagaagacggcatacgagatgcacataggtctcgtgggctcggagatgtgtataagagacag

SEQ ID NO: 128 - DNA, 66 bp, synthetic construct
  misc_feature 1..66: note = Synthetic DNA
  source 1..66: mol_type = other DNA
caagcagaagacggcatacgagatatgatgaggtctcgtgggctcggagatgtgtataagagacag

SEQ ID NO: 129 - DNA, 66 bp, synthetic construct
  misc_feature 1..66: note = Synthetic DNA
  source 1..66: mol_type = other DNA
caagcagaagacggcatacgagatagtagtaggtctcgtgggctcggagatgtgtataagagacag

SEQ ID NO: 130 - DNA, 66 bp, synthetic construct
  misc_feature 1..66: note = Synthetic DNA
  source 1..66: mol_type = other DNA
caagcagaagacggcatacgagattatgtcaggtctcgtgggctcggagatgtgtataagagacag

SEQ ID NO: 131 - DNA, 66 bp, synthetic construct
  misc_feature 1..66: note = Synthetic DNA
  source 1..66: mol_type = other DNA
caagcagaagacggcatacgagatgtgtgtgagtctcgtgggctcggagatgtgtataagagacag

SEQ ID NO: 132 - DNA, 66 bp, synthetic construct
  misc_feature 1..66: note = Synthetic DNA
  source 1..66: mol_type = other DNA
caagcagaagacggcatacgagattacgacaggtctcgtgggctcggagatgtgtataagagacag

SEQ ID NO: 133 - DNA, 66 bp, synthetic construct
  misc_feature 1..66: note = Synthetic DNA
  source 1..66: mol_type = other DNA
caagcagaagacggcatacgagatatgtgatggtctcgtgggctcggagatgtgtataagagacag

SEQ ID NO: 134 - DNA, 70 bp, synthetic construct
  misc_feature 1..70: note = Synthetic DNA
  source 1..70: mol_type = other DNA
aatgatacggcgaccaccgagatctacaccgcgcagatcgtcggcagcgtcagatgtgtataagagacag

SEQ ID NO: 135 - DNA, 162 bp, Saccharum spontaneum
  source 1..162: mol_type = genomic DNA
cagaacgtggtcgccgtcgccatgctccttcgtaacatgcctgagccatcgaatcctgacactcgtcaagcccgagatgaaatccgaggactcatcgagaccgctgctatgcagcaaaccgagagttctgccttgaggtgacgcgggcctacctcggagcag

SEQ ID NO: 136 - DNA, 128 bp, Saccharum spontaneum
  source 1..128: mol_type = genomic DNA
ctctggcgaacgcggtgaggagcacacccatggtctgccccggcgagatgaccagcgtggtggccgctgtagacccccacacgccgccctgcactctgcctccttccgaggagttcgacctcttcggc

SEQ ID NO: 137 - DNA, 216 bp, Saccharum spontaneum
  source 1..216: mol_type = genomic DNA
gagcacttgatgtcacctaatatactggttctgagccattacccgtttggggtccaattaatttatgaaatgacttgttgtcaattaccctgagtttttgtccactctgaagctgaactgaacattgtgtgctccggtagctgaaaatcctacccttagcactcagactgagtccattgttcttatcatagccttatggttgttcattttgtatgc

SEQ ID NO: 138 - DNA, 238 bp, Saccharum spontaneum
  source 1..238: mol_type = genomic DNA
ctcgagcggtgtccgacaggaacagggggagttgtcgtatgatgacccggtcgtcagtggccccacccaactgacaagccaagtggaagtcagcgagccaaagtttgggtttggtttcgccgttatacttggtaagagtcgtgggggggcggaaccgagccgggagtaacatttcatgaatggccttgctaaagacccgaggtcccggtggctcgggtgaagggctttgatcctccga

SEQ ID NO: 139 - DNA, 212 bp, Saccharum spontaneum
  source 1..212: mol_type = genomic DNA
cagtaagctccatcgagggacaagcatctagtgagtcaataccaaagtggcgtggggcataaccatacagagcctgaaatggggaacgatccagagtggaatgccaacttgaattgtaccaaaactcggcaagatggatccaattatgccacttgtggggtaccgcattgacaaaacagcgtaagaaggtctccatgcactggttgacgcgc

SEQ ID NO: 140 - DNA, 239 bp, Saccharum spontaneum
  source 1..239: mol_type = genomic DNA
acagtgaacagtggtactatatacagtacccgctacagcaacacacgtcgtggtctccttaggcagcagaaatggtataaaaaaacgtacataaaggtacatggcagataggagtatattctataaggagcacgtaacgcgtgacgggtcacgcgttccagcgtcgtctacctcgtgacaggcctcctcccttgcccccttctccctccccgtgcccctcctcctctgccccctccctc

SEQ ID NO: 141 - DNA, 124 bp, Saccharum spontaneum
  source 1..124: mol_type = genomic DNA
caaaacagggacaatatatatcctttgtctgcttaatcaaagcatgccttttagctgcttgaataaaaccattcacatcttgtatgtagaaagcttcatatcttcgtttcgtgtacatccatga

SEQ ID NO: 142 - DNA, 197 bp, Saccharum spontaneum
  source 1..197: mol_type = genomic DNA
gccaccggcctcgtggtcgtgagaccgaggagaccggtacctggggatgggctggtagtcctcatcagtgctgtctgaagtgacatcctcaggtatatcagcagtctgagtctcaaggtccgcgagctctgcctcggcaagctcgtcaactctggcgtcctgctcagctgctgtctcaggaacatctgggcgctcac

SEQ ID NO: 143 - DNA, 140 bp, Saccharum spontaneum
  source 1..140: mol_type = genomic DNA
cagaggcccttgaaagcacagctggttttcatcgtcacccagtgaagaatgacatttcgaggaaggctttaggctacttgaatcccacgaacatttctacagtaatattggtaatatgcgcagagggtccttggctactc

SEQ ID NO: 144 - DNA, 100 bp, Saccharum spontaneum
  source 1..100: mol_type = genomic DNA
gatcccggaggaagttcaggaccggccagagcagccagaggtaccaaccggcgacgaagctcctgccgaagatctacccgagtgtcctgaccaccggccc

SEQ ID NO: 145 - DNA, 100 bp, Saccharum spontaneum
  source 1..100: mol_type = genomic DNA
tacaatgcagcaagtgttttcatataactcctagaacttaatgcaataaacaattaaaagtactcaagatagatttataaaaatgatgggttcagcatgc

SEQ ID NO: 146 - DNA, 175 bp, Saccharum spontaneum
  source 1..175: mol_type = genomic DNA
gcggaaaatagaccaatgtattaggctatccattgccgatatgcaaaagaatgaatccataatgatagccgccgctcacttctggtcagacaccacaaatacctttatgtttggtcatggtccatccgcccctactcttgccgatgtatacatgctcaccgacttagatatctca

SEQ ID NO: 147 - DNA, 81 bp, Saccharum spontaneum
  source 1..81: mol_type = genomic DNA
caggaacttgcaaacaagctcgggtgcacagatccaaatcatttggtcggttgatgtggactccgatgtggactcaaatgc

SEQ ID NO: 148 - DNA, 136 bp, Saccharum spontaneum
  source 1..136: mol_type = genomic DNA
gcgtggcagaggatgggggcacgacagggatggcaccgtggcgaggggaggagcacggtgtggcagggaaaggagcgcggcgagcttgacgccgggggcgcaaccaggatgaggattgagtcacggatggttggac

SEQ ID NO: 149 - DNA, 135 bp, Saccharum spontaneum
  source 1..135: mol_type = genomic DNA
gaaaacaagctctaccactggcacaacacgggctcacaggtcacagctctgcttgctcagtggtactacgagtgctagcacgggagcgctgttggacggatcgccagcatgaaatgcccaccgcgcacgtactgc

SEQ ID NO: 150 - DNA, 143 bp, Saccharum spontaneum
  source 1..143: mol_type = genomic DNA
taccccaccaaaaagcctatcatgatccttggtgtcattttatttttggttctgtcaggtaagtctagctgagtaccttctcatactcagggctttgttcccacttgttacagatggacagatgtactatggttattgtatca

SEQ ID NO: 151 - DNA, 146 bp, Saccharum spontaneum
  source 1..146: mol_type = genomic DNA
taacccaccaaagatagccttgcatgctccttagtgtcgcttattttggtttaagacgggtaagtctagctgagtaccttctcgtactcagggcgttgttctcattgttgttgcagatggttagatgtactatggatattgcgtca

SEQ ID NO: 152 - DNA, 66 bp, synthetic construct; identical to SEQ ID NO: 19
  misc_feature 1..66: note = Synthetic DNA
  source 1..66: mol_type = other DNA

SEQ ID NO: 153 - DNA, 66 bp, synthetic construct; identical to SEQ ID NO: 20
  misc_feature 1..66: note = Synthetic DNA
  source 1..66: mol_type = other DNA

SEQ ID NO: 154 - DNA, 66 bp, synthetic construct; identical to SEQ ID NO: 21
  misc_feature 1..66: note = Synthetic DNA
  source 1..66: mol_type = other DNA

SEQ ID NO: 155 - DNA, 66 bp, synthetic construct; identical to SEQ ID NO: 22
  misc_feature 1..66: note = Synthetic DNA
  source 1..66: mol_type = other DNA

SEQ ID NO: 156 - DNA, 66 bp, synthetic construct; identical to SEQ ID NO: 24
  misc_feature 1..66: note = Synthetic DNA
  source 1..66: mol_type = other DNA

SEQ ID NO: 157 - DNA, 66 bp, synthetic construct; identical to SEQ ID NO: 25
  misc_feature 1..66: note = Synthetic DNA
  source 1..66: mol_type = other DNA

SEQ ID NO: 158 - DNA, 66 bp, synthetic construct; identical to SEQ ID NO: 26
  misc_feature 1..66: note = Synthetic DNA
  source 1..66: mol_type = other DNA

SEQ ID NO: 159 - DNA, 66 bp, synthetic construct; identical to SEQ ID NO: 29
  misc_feature 1..66: note = Synthetic DNA
  source 1..66: mol_type = other DNA

SEQ ID NO: 160 - DNA, 66 bp, synthetic construct; identical to SEQ ID NO: 30
  misc_feature 1..66: note = Synthetic DNA
  source 1..66: mol_type = other DNA

SEQ ID NO: 161 - DNA, 66 bp, synthetic construct; identical to SEQ ID NO: 31
  misc_feature 1..66: note = Synthetic DNA
  source 1..66: mol_type = other DNA

SEQ ID NO: 162 - DNA, 66 bp, synthetic construct; identical to SEQ ID NO: 32
  misc_feature 1..66: note = Synthetic DNA
  source 1..66: mol_type = other DNA

SEQ ID NO: 163 - DNA, 66 bp, synthetic construct; identical to SEQ ID NO: 33
  misc_feature 1..66: note = Synthetic DNA
  source 1..66: mol_type = other DNA

SEQ ID NO: 164 - DNA, 66 bp, synthetic construct; identical to SEQ ID NO: 34
  misc_feature 1..66: note = Synthetic DNA
  source 1..66: mol_type = other DNA

SEQ ID NO: 165 - DNA, 66 bp, synthetic construct; identical to SEQ ID NO: 35
  misc_feature 1..66: note = Synthetic DNA
  source 1..66: mol_type = other DNA

SEQ ID NO: 166 - DNA, 66 bp, synthetic construct; identical to SEQ ID NO: 36
  misc_feature 1..66: note = Synthetic DNA
  source 1..66: mol_type = other DNA

SEQ ID NO: 167 - DNA, 66 bp, synthetic construct; identical to SEQ ID NO: 37
  misc_feature 1..66: note = Synthetic DNA
  source 1..66: mol_type = other DNA

SEQ ID NO: 168 - DNA, 66 bp, synthetic construct; identical to SEQ ID NO: 38
  misc_feature 1..66: note = Synthetic DNA
  source 1..66: mol_type = other DNA

SEQ ID NO: 169 - DNA, 66 bp, synthetic construct; identical to SEQ ID NO: 39
  misc_feature 1..66: note = Synthetic DNA
  source 1..66: mol_type = other DNA

SEQ ID NO: 170 - DNA, 66 bp, synthetic construct; identical to SEQ ID NO: 40
  misc_feature 1..66: note = Synthetic DNA
  source 1..66: mol_type = other DNA

SEQ ID NO: 171 - DNA, 66 bp, synthetic construct; identical to SEQ ID NO: 41
  misc_feature 1..66: note = Synthetic DNA
  source 1..66: mol_type = other DNA

SEQ ID NO: 172 - DNA, 66 bp, synthetic construct; identical to SEQ ID NO: 42
  misc_feature 1..66: note = Synthetic DNA
  source 1..66: mol_type = other DNA

SEQ ID NO: 173 - DNA, 66 bp, synthetic construct; identical to SEQ ID NO: 43
  misc_feature 1..66: note = Synthetic DNA
  source 1..66: mol_type = other DNA

SEQ ID NO: 174 - DNA, 66 bp, synthetic construct; identical to SEQ ID NO: 44
  misc_feature 1..66: note = Synthetic DNA
  source 1..66: mol_type = other DNA

SEQ ID NO: 175 - DNA, 66 bp, synthetic construct; identical to SEQ ID NO: 45
  misc_feature 1..66: note = Synthetic DNA
  source 1..66: mol_type = other DNA

SEQ ID NO: 176 - DNA, 66 bp, synthetic construct; identical to SEQ ID NO: 46
  misc_feature 1..66: note = Synthetic DNA
  source 1..66: mol_type = other DNA

SEQ ID NO: 177 - DNA, 66 bp, synthetic construct; identical to SEQ ID NO: 47
  misc_feature 1..66: note = Synthetic DNA
  source 1..66: mol_type = other DNA

SEQ ID NO: 178 - DNA, 66 bp, synthetic construct; identical to SEQ ID NO: 48
  misc_feature 1..66: note = Synthetic DNA
  source 1..66: mol_type = other DNA

SEQ ID NO: 179 - DNA, 66 bp, synthetic construct; identical to SEQ ID NO: 49
  misc_feature 1..66: note = Synthetic DNA
  source 1..66: mol_type = other DNA

SEQ ID NO: 180 - DNA, 66 bp, synthetic construct; identical to SEQ ID NO: 50
  misc_feature 1..66: note = Synthetic DNA
  source 1..66: mol_type = other DNA

SEQ ID NO: 181 - DNA, 66 bp, synthetic construct; identical to SEQ ID NO: 51
  misc_feature 1..66: note = Synthetic DNA
  source 1..66: mol_type = other DNA

SEQ ID NO: 182 - DNA, 66 bp, synthetic construct; identical to SEQ ID NO: 52
  misc_feature 1..66: note = Synthetic DNA
  source 1..66: mol_type = other DNA

SEQ ID NO: 183 - DNA, 66 bp, synthetic construct; identical to SEQ ID NO: 53
  misc_feature 1..66: note = Synthetic DNA
  source 1..66: mol_type = other DNA

SEQ ID NO: 184 - DNA, 66 bp, synthetic construct; identical to SEQ ID NO: 54
  misc_feature 1..66: note = Synthetic DNA
  source 1..66: mol_type = other DNA

SEQ ID NO: 185 - DNA, 66 bp, synthetic construct; identical to SEQ ID NO: 55
  misc_feature 1..66: note = Synthetic DNA
  source 1..66: mol_type = other DNA

SEQ ID NO: 186 - DNA, 66 bp, synthetic construct; identical to SEQ ID NO: 56
  misc_feature 1..66: note = Synthetic DNA
  source 1..66: mol_type = other DNA

SEQ ID NO: 187 - DNA, 66 bp, synthetic construct; identical to SEQ ID NO: 57
  misc_feature 1..66: note = Synthetic DNA
  source 1..66: mol_type = other DNA

SEQ ID NO: 188 - DNA, 66 bp, synthetic construct; identical to SEQ ID NO: 58
  misc_feature 1..66: note = Synthetic DNA
  source 1..66: mol_type = other DNA

SEQ ID NO: 189 - DNA, 66 bp, synthetic construct; identical to SEQ ID NO: 59
  misc_feature 1..66: note = Synthetic DNA
  source 1..66: mol_type = other DNA

SEQ ID NO: 190 - DNA, 66 bp, synthetic construct; identical to SEQ ID NO: 60
  misc_feature 1..66: note = Synthetic DNA
  source 1..66: mol_type = other DNA

SEQ ID NO: 191 - DNA, 66 bp, synthetic construct; identical to SEQ ID NO: 61
  misc_feature 1..66: note = Synthetic DNA
  source 1..66: mol_type = other DNA

SEQ ID NO: 192 - DNA, 66 bp, synthetic construct; identical to SEQ ID NO: 62
  misc_feature 1..66: note = Synthetic DNA
  source 1..66: mol_type = other DNA

SEQ ID NO: 193 - DNA, 66 bp, synthetic construct; identical to SEQ ID NO: 63
  misc_feature 1..66: note = Synthetic DNA
  source 1..66: mol_type = other DNA

SEQ ID NO: 194 - DNA, 66 bp, synthetic construct; identical to SEQ ID NO: 64
  misc_feature 1..66: note = Synthetic DNA
  source 1..66: mol_type = other DNA

SEQ ID NO: 195 - DNA, 66 bp, synthetic construct; identical to SEQ ID NO: 65
  misc_feature 1..66: note = Synthetic DNA
  source 1..66: mol_type = other DNA

SEQ ID NO: 196 - DNA, 66 bp, synthetic construct; identical to SEQ ID NO: 66
  misc_feature 1..66: note = Synthetic DNA
  source 1..66: mol_type = other DNA

SEQ ID NO: 197 - DNA, 66 bp, synthetic construct; identical to SEQ ID NO: 67
  misc_feature 1..66: note = Synthetic DNA
  source 1..66: mol_type = other DNA

SEQ ID NO: 198 - DNA, 66 bp, synthetic construct; identical to SEQ ID NO: 68
  misc_feature 1..66: note = Synthetic DNA
  source 1..66: mol_type = other DNA

SEQ ID NO: 199 - DNA, 66 bp, synthetic construct; identical to SEQ ID NO: 69
  misc_feature 1..66: note = Synthetic DNA
  source 1..66: mol_type = other DNA

SEQ ID NO: 200 - DNA, 66 bp, synthetic construct; identical to SEQ ID NO: 70
  misc_feature 1..66: note = Synthetic DNA
  source 1..66: mol_type = other DNA

SEQ ID NO: 201 - DNA, 66 bp, synthetic construct; identical to SEQ ID NO: 71
  misc_feature 1..66: note = Synthetic DNA
  source 1..66: mol_type = other DNA

SEQ ID NO: 202 - DNA, 66 bp, synthetic construct; identical to SEQ ID NO: 72
  misc_feature 1..66: note = Synthetic DNA
  source 1..66: mol_type = other DNA

SEQ ID NO: 203 - DNA, 66 bp, synthetic construct; identical to SEQ ID NO: 73
  misc_feature 1..66: note = Synthetic DNA
  source 1..66: mol_type = other DNA

SEQ ID NO: 204 - DNA, 66 bp, synthetic construct; identical to SEQ ID NO: 74
  misc_feature 1..66: note = Synthetic DNA
  source 1..66: mol_type = other DNA

SEQ ID NO: 205 - DNA, 66 bp, synthetic construct; identical to SEQ ID NO: 75
  misc_feature 1..66: note = Synthetic DNA
  source 1..66: mol_type = other DNA

SEQ ID NO: 206 - DNA, 66 bp, synthetic construct; identical to SEQ ID NO: 76
  misc_feature 1..66: note = Synthetic DNA
  source 1..66: mol_type = other DNA

SEQ ID NO: 207 - DNA, 66 bp, synthetic construct; identical to SEQ ID NO: 77
  misc_feature 1..66: note = Synthetic DNA
  source 1..66: mol_type = other DNA

SEQ ID NO: 208 - DNA, 66 bp, synthetic construct; identical to SEQ ID NO: 78
  misc_feature 1..66: note = Synthetic DNA
  source 1..66: mol_type = other DNA

SEQ ID NO: 209 - DNA, 66 bp, synthetic construct; identical to SEQ ID NO: 79
  misc_feature 1..66: note = Synthetic DNA
  source 1..66: mol_type = other DNA

SEQ ID NO: 210 - DNA, 66 bp, synthetic construct; identical to SEQ ID NO: 80
  misc_feature 1..66: note = Synthetic DNA
  source 1..66: mol_type = other DNA

SEQ ID NO: 211 - DNA, 66 bp, synthetic construct; identical to SEQ ID NO: 81
  misc_feature 1..66: note = Synthetic DNA
  source 1..66: mol_type = other DNA

SEQ ID NO: 212 - DNA, 66 bp, synthetic construct; identical to SEQ ID NO: 82
  misc_feature 1..66: note = Synthetic DNA
  source 1..66: mol_type = other DNA

SEQ ID NO: 213 - DNA, 66 bp, synthetic construct; identical to SEQ ID NO: 83
  misc_feature 1..66: note = Synthetic DNA
  source 1..66: mol_type = other DNA

SEQ ID NO: 214 - DNA, 66 bp, synthetic construct; identical to SEQ ID NO: 84
  misc_feature 1..66: note = Synthetic DNA
  source 1..66: mol_type = other DNA

SEQ ID NO: 215 - DNA, 66 bp, synthetic construct; identical to SEQ ID NO: 85
  misc_feature 1..66: note = Synthetic DNA
  source 1..66: mol_type = other DNA

SEQ ID NO: 216 - DNA, 66 bp, synthetic construct; identical to SEQ ID NO: 86
  misc_feature 1..66: note = Synthetic DNA
  source 1..66: mol_type = other DNA

SEQ ID NO: 217 - DNA, 66 bp, synthetic construct; identical to SEQ ID NO: 87
  misc_feature 1..66: note = Synthetic DNA
  source 1..66: mol_type = other DNA

SEQ ID NO: 218 - DNA, 66 bp, synthetic construct; identical to SEQ ID NO: 88
  misc_feature 1..66: note = Synthetic DNA
  source 1..66: mol_type = other DNA

SEQ ID NO: 219 - DNA, 66 bp, synthetic construct; identical to SEQ ID NO: 89
  misc_feature 1..66: note = Synthetic DNA
  source 1..66: mol_type = other DNA

SEQ ID NO: 220 - DNA, 66 bp, synthetic construct; identical to SEQ ID NO: 90
  misc_feature 1..66: note = Synthetic DNA
  source 1..66: mol_type = other DNA

SEQ ID NO: 221 - DNA, 66 bp, synthetic construct; identical to SEQ ID NO: 91
  misc_feature 1..66: note = Synthetic DNA
  source 1..66: mol_type = other DNA

SEQ ID NO: 222 - DNA, 66 bp, synthetic construct; identical to SEQ ID NO: 92
  misc_feature 1..66: note = Synthetic DNA
  source 1..66: mol_type = other DNA

SEQ ID NO: 223 - DNA, 66 bp, synthetic construct; identical to SEQ ID NO: 93
  misc_feature 1..66: note = Synthetic DNA
  source 1..66: mol_type = other DNA

SEQ ID NO: 224 - DNA, 66 bp, synthetic construct; identical to SEQ ID NO: 94
  misc_feature 1..66: note = Synthetic DNA
  source 1..66: mol_type = other DNA

SEQ ID NO: 225 - DNA, 66 bp, synthetic construct; identical to SEQ ID NO: 95
  misc_feature 1..66: note = Synthetic DNA
  source 1..66: mol_type = other DNA

SEQ ID NO: 226 - DNA, 66 bp, synthetic construct; identical to SEQ ID NO: 96
  misc_feature 1..66: note = Synthetic DNA
  source 1..66: mol_type = other DNA

SEQ ID NO: 227 - DNA, 66 bp, synthetic construct; identical to SEQ ID NO: 97
  misc_feature 1..66: note = Synthetic DNA
  source 1..66: mol_type = other DNA

SEQ ID NO: 228 - DNA, 66 bp, synthetic construct; identical to SEQ ID NO: 98
  misc_feature 1..66: note = Synthetic DNA
  source 1..66: mol_type = other DNA

SEQ ID NO: 229 - DNA, 66 bp, synthetic construct; identical to SEQ ID NO: 99
  misc_feature 1..66: note = Synthetic DNA
  source 1..66: mol_type = other DNA

SEQ ID NO: 230 - DNA, 66 bp, synthetic construct; identical to SEQ ID NO: 100
  misc_feature 1..66: note = Synthetic DNA
  source 1..66: mol_type = other DNA

SEQ ID NO: 231 - DNA, 66 bp, synthetic construct; identical to SEQ ID NO: 101
  misc_feature 1..66: note = Synthetic DNA
  source 1..66: mol_type = other DNA

SEQ ID NO: 232 - DNA, 66 bp, synthetic construct; identical to SEQ ID NO: 102
  misc_feature 1..66: note = Synthetic DNA
  source 1..66: mol_type = other DNA

SEQ ID NO: 233 - DNA, 66 bp, synthetic construct; identical to SEQ ID NO: 103
  misc_feature 1..66: note = Synthetic DNA
  source 1..66: mol_type = other DNA

SEQ ID NO: 234 - DNA, 66 bp, synthetic construct; identical to SEQ ID NO: 105
  misc_feature 1..66: note = Synthetic DNA
  source 1..66: mol_type = other DNA

SEQ ID NO: 235 - DNA, 66 bp, synthetic construct; identical to SEQ ID NO: 107
  misc_feature 1..66: note = Synthetic DNA
  source 1..66: mol_type = other DNA

SEQ ID NO: 236 - DNA, 66 bp, synthetic construct; identical to SEQ ID NO: 108
  misc_feature 1..66: note = Synthetic DNA
  source 1..66: mol_type = other DNA

SEQ ID NO: 237 - DNA, 66 bp, synthetic construct; identical to SEQ ID NO: 109
  misc_feature 1..66: note = Synthetic DNA
  source 1..66: mol_type = other DNA

SEQ ID NO: 238 - DNA, 66 bp, synthetic construct; identical to SEQ ID NO: 110
  misc_feature 1..66: note = Synthetic DNA
  source 1..66: mol_type = other DNA

SEQ ID NO: 239 - DNA, 66 bp, synthetic construct; identical to SEQ ID NO: 111
  misc_feature 1..66: note = Synthetic DNA
  source 1..66: mol_type = other DNA

SEQ ID NO: 240 - DNA, 66 bp, synthetic construct; identical to SEQ ID NO: 112
  misc_feature 1..66: note = Synthetic DNA
  source 1..66: mol_type = other DNA

SEQ ID NO: 241 - DNA, 66 bp, synthetic construct; identical to SEQ ID NO: 113
  misc_feature 1..66: note = Synthetic DNA
  source 1..66: mol_type = other DNA

SEQ ID NO: 242 - DNA, 66 bp, synthetic construct; identical to SEQ ID NO: 114
  misc_feature 1..66: note = Synthetic DNA
  source 1..66: mol_type = other DNA

SEQ ID NO: 243 - DNA, 126 bp, Saccharum spontaneum
  source 1..126: mol_type = genomic DNA
acagggaagtgaaagagaagtggccattgaaaaacctaatattgttacggaagtgcctcgcgtccgcgtcgaagctcgtccaaagcctcatgagctggtctggtatgactggcgcattcagctcga

SEQ ID NO: 244 - DNA, 89 bp, Saccharum spontaneum
  source 1..89: mol_type = genomic DNA
caagcgcaggaaaaggaaacgctgcccgcagttcctcacccggctcccccgttgtcgccctcagccgcttccgccatcctcgctgtcac

SEQ ID NO: 245 - DNA, 112 bp, Saccharum spontaneum
  source 1..112: mol_type = genomic DNA
caataaaatgggaacatgatggaaggacaggttacccgatgcattatgagcatacagaacggcctacaaaacaccgattggaatgtcaccatatcaacttatctatggaaga

SEQ ID NO: 246 - DNA, 156 bp, Saccharum spontaneum
  source 1..156: mol_type = genomic DNA
cagacagatagccacgagacgaggactcacagagctttcggtgagcctccatagtccagagatagtctctgttggttctttttctttcctttttcgacgagatctttttttcttggtcccacacgaactggcgttgtgcttctattatgctgtcac

SEQ ID NO: 247 - DNA, 121 bp, Saccharum spontaneum
  source 1..121: mol_type = genomic DNA
catcatggtgcgaatcttcgagttcagacccctcataaaacaagcttgcttctttcgatctgagttgatttggtcagctgcatactatgataaatgattgaacctacccacatattgcatc

SEQ ID NO: 248 - DNA, 126 bp, Saccharum spontaneum
  source 1..126: mol_type = genomic DNA
catgaaagctcacaaacccgctcgagctctgggtcgatggctcatcccatttgcctcgagaatgcccctcctccgctgcgcaggtgccgcctctccttacatcacgcgtcaccatgccatgcccgc

SEQ ID NO: 249 - DNA, 100 bp, Saccharum spontaneum
  source 1..100: mol_type = genomic DNA
ctaatgcttggtagctcatacatgaaccttatagtcgtcctgcatttaacaactgtcaaggacaggttcgagtgcacatgtataatgctatcaacgtttt

SEQ ID NO: 250 - DNA, 100 bp, Saccharum spontaneum
  source 1..100: mol_type = genomic DNA
tagccacataactttcccacaatgtaaaggtattttcaataaagaatctaggagtactaattctatgttttcaatctatacaatcattggcatattgatg

SEQ ID NO: 251 - DNA, 153 bp, Saccharum spontaneum
  source 1..153: mol_type = genomic DNA
ctcgaaaatctctaacaaactcattccaagtaataggaggagcattgttaggacgtgcagcttgatacgactcccaccaagtctgtgttgtcccctgtaactgaccagttgcatataacaccttttccatatcattgcattgagcgatgttca

SEQ ID NO: 252 - DNA, 100 bp, Saccharum spontaneum
  source 1..100: mol_type = genomic DNA
ctcgagcgatgtccgataggaacaggggaagttgtcgaatgatgacccggtcgtcggtggccccacccaactgactagccaagcggaagtcagcgagcca

SEQ ID NO: 253 - DNA, 100 bp, Saccharum spontaneum
  source 1..100: mol_type = genomic DNA
tcggaggatcgaagcccttcacccgagccaccaggccctcgggtctttagcaaagccatccgcaaaacgttactcccagctcggttcctcccccatgact

SEQ ID NO: 254 - DNA, 100 bp, Saccharum spontaneum
  source 1..100: mol_type = genomic DNA
ctcgagcggtgtcagagaggaacagggggagctgccggatgatgttccggtcgtcggtggccccgcccagctgacacgccaagcggaaatcggcgagcca

SEQ ID NO: 255 - DNA, 100 bp, Saccharum spontaneum
  source 1..100: mol_type = genomic DNA
tcggaggatcgtagcccttcacccgagcctccgggccctcgggtcttcagtaaggctattcgtgaaatgatactcccggcgcggtttcgcccccccccca

SEQ ID NO: 256 - DNA, 100 bp, Saccharum spontaneum
  source 1..100: mol_type = genomic DNA
ctcgagcggtgtcgaagaggaacagagggagctgtcggatgatgacctggtcgtcggtggccccacccagctgacatgccaagcggaaatcggcgagcca

SEQ ID NO: 257 - DNA, 100 bp, Saccharum spontaneum
  source 1..100: mol_type = genomic DNA
tcggaggatcgcagcccatcacccgagcctccaggccctcgggtcttcagtaaggctatccgcgaaactatactcccggcgcagtttcgccctcccacga

SEQ ID NO: 258 - DNA, 100 bp, Saccharum spontaneum
  source 1..100: mol_type = genomic DNA
ctcgagcggtgtcggagaggaacagggggagctgtcggatgatgacccggtcgtcggtggccccacccagctgacacgccaagcggaaatcggcgagcca

SEQ ID NO: 259 - DNA, 100 bp, Saccharum spontaneum
  source 1..100: mol_type = genomic DNA
tcggaggatcgtagcccttcacccgagcctccgggccctcgggtcttcagtaaggctatccgcgaaataatactcccggcgcggtttcgccctcccacga

SEQ ID NO: 260 - DNA, 100 bp, Saccharum spontaneum
  source 1..100: mol_type = genomic DNA
ctcgagcggtgtcggagaggaacagggggagctgtcggatgatgacccggtcgtcggtggccccgcccagctgacatgccaagcggaaatcggcgagcca

SEQ ID NO: 261 - DNA, 100 bp, Saccharum spontaneum
  source 1..100: mol_type = genomic DNA
tcggaggatcgaagcccttcacccgagcctccgggccctcgggtcttcagcaaggctatccgcgaaacgatactcctggcgcggtttcgccctcccacga

SEQ ID NO: 262 - DNA, 100 bp, Saccharum spontaneum
  source 1..100: mol_type = genomic DNA
ctcgagcggtgttagagaggaacagggggagctgtcggatgatgacccggtcgtcggtggccccacccagctgacatgccaagcggaaatcggcaagcca

SEQ ID NO: 263 - DNA, 100 bp, Saccharum spontaneum
  source 1..100: mol_type = genomic DNA
tcggaggatcgtagcccttcacccgagcctccgggccctcgggtcttcagtaaggctatccgcgaaacgatactcccggcgcggtttcgccctcccacga

SEQ ID NO: 264 - DNA, 100 bp, Saccharum spontaneum
  source 1..100: mol_type = genomic DNA
ctcgggcggtatcggagaggaatagggggagttgtcggatgatgacccggtcgtcggtggccccgcctagctaacacgccaagcagaaatcggcgagcca

SEQ ID NO: 265 - DNA, 100 bp, Saccharum spontaneum
  source 1..100: mol_type = genomic DNA
tcagaagatcgtagcccttcacccgagcctccgggctctcgggtctttagtaaggctatccgcgagacggtgctcccggctcggtttcgccctcccacga

SEQ ID NO: 266 - DNA, 100 bp, Saccharum spontaneum
  source 1..100: mol_type = genomic DNA
ctcgggcggtgtcggagaggaacagggggagttatcgaatgatgacccggtcgtcggtggccccgcccagctgacacgccaagcggaaatcggtgagcca

SEQ ID NO: 267 - DNA, 100 bp, Saccharum spontaneum
  source 1..100: mol_type = genomic DNA
tcggaagatcgtagtccttcacccgagcctccgggccctctggtcttctgtaaagctatccgtgaaatggtgctcccggctcagtttcgccctcccacga

SEQ ID NO: 268 - DNA, 100 bp, Saccharum spontaneum
  source 1..100: mol_type = genomic DNA
ctcgggcggtgtcggagaggaacagggggagttgtcgaatgataacccggtcgtcggtggccccacccagctgacatgccaagcggaaatcagcaagcca

SEQ ID NO: 269 - DNA, 100 bp, Saccharum spontaneum
  source 1..100: mol_type = genomic DNA
tcgaaagatcgtagtccttcacccgagcctccgggccctcgggtcttcagtaaggctatccgtgaaacggtgctcccatctcggtttcgccctcccacga

SEQ ID NO: 270 - DNA, 170 bp, Saccharum spontaneum
  source 1..170: mol_type = genomic DNA
ctgagtctagtaataagactttgatcaagcttatcaagaagaaaattgaggaaaattcaaggaggtggcatgaagttttgtctgaagctctatgggcacatcgtatttcaaaacatggtgctaccaaagttattccttttaagctagtatatgaccaagaggccgtgtta

SEQ ID NO: 271 - DNA, 84 bp, Saccharum spontaneum
  source 1..84: mol_type = genomic DNA
gaagctactgttggttgggagctaatcggaatgactatttatcattttttgaaaataaaaagggatgactgatacttatgcaca

SEQ ID NO: 272 - DNA, 85 bp, Saccharum spontaneum
  source 1..85: mol_type = genomic DNA
gacaatgcatcacgccgatgtagccgagatcatcccttcgctgcccctggagatcaggtggccaccgttccgtctccgccattac

SEQ ID NO: 273 - DNA, 160 bp, Saccharum spontaneum
  source 1..160: mol_type = genomic DNA
gaccacgcgggcgacatcgacaccagcaagagcacgagcgggattctcttcttcctcggcaggtgcctcgttagctggcagtcggtcaagcagcaggtggtggccctgtccagctgcgaggccgagtacatagcggcttccaccgcttcgacttaggcgc

SEQ ID NO: 274 - DNA, 104 bp, Saccharum spontaneum
  source 1..104: mol_type = genomic DNA
gacctggcaggcgacgtgaatgactgaaagagcaccagcgggctgatcttcttcctggcaggaggcccgattgcttggcagtcggcaaaacagagggtggttgc

SEQ ID NO: 275 - DNA, 100 bp, Saccharum spontaneum
  source 1..100: mol_type = genomic DNA
gagatgagaggagcgaggaaaggagaggaagggaggtgaggtgttgtagtgtggatacagctaacagcttacatggaggtggagtatggggcctggttta

SEQ ID NO: 276 - DNA, 100 bp, Saccharum spontaneum
  source 1..100: mol_type = genomic DNA
gcgcaggaaagaggtcgtggacaaatccagggcgtgtttagttgggctgattttggacgtccaaaatctgcacagtgtaagattccatactgtagcattt

SEQ ID NO: 277 - DNA, 100 bp, Saccharum spontaneum
  source 1..100: mol_type = genomic DNA
gagcgagacgagagacaaacaaaaaggtggtgacgataagaggcgccacgcactaaacccgcaccatggtcatggagtagcgtgtaaggaacaagtagca

SEQ ID NO: 278 - DNA, 100 bp, Saccharum spontaneum
  source 1..100: mol_type = genomic DNA
gcgagaggtggtggtacacgcctacctaggtggtgggctcgtcgtcgtcagacaccaggcgtcacaggccgtcgtccgtttctgccgccccatgctcgct

SEQ ID NO: 279 - DNA, 100 bp, Saccharum spontaneum
  source 1..100: mol_type = genomic DNA
gcacgacatggtatggcaggggacaaagacggagatggggtgcgccgtgggggatggcttcagcgacatgccagagtgggccacggcgcgacagagtggg

SEQ ID NO: 280 - DNA, 100 bp, Saccharum spontaneum
  source 1..100: mol_type = genomic DNA
gctgctcccttctccggcgcgaggctcctcgtcgtggccttctctagcgcggtggcaccgcatccgctcccatcccgcgctgctccacactccgcgcgcg

SEQ ID NO: 281 - DNA, 100 bp, Saccharum spontaneum
  source 1..100: mol_type = genomic DNA
gccaccggcctcgtggtcgtgagaccgaggagaccggtacctgggatgggctggtagtcctcatctgtgctgtcagaagtcacatcctcaggtatatcag

SEQ ID NO: 282 - DNA, 100 bp, Saccharum spontaneum
  source 1..100: mol_type = genomic DNA
gtgagcgcccagacgttcctgagacagcagctgagcaggatgccagagttgacgagcttgctgaggcagagcttgcggaccttgaggctcagactgctga

SEQ ID NO: 283 - DNA, 130 bp, Saccharum spontaneum
  source 1..130: mol_type = genomic DNA
gcggttgtgtgttgtggccgggccattggcggatgagcgagcgagtgcaaccagaggcgaacgtacccaacccagcgacagcaagtcagcaacatatattctctcactcgatcacgtccccacgggccta

SEQ ID NO: 284 - DNA, 151 bp, Saccharum spontaneum
  source 1..151: mol_type = genomic DNA
gctaagaacattcggtgtctgcaatgtgagaagtgaacactctagtaacttaagaaatgctatcaacaaaatgagccatcttgttcatctagacattgcggctctaggggagagcgaagtgttgcagctagagggacttcatttgcctcca

SEQ ID NO: 285 - DNA, 100 bp, Saccharum spontaneum
  source 1..100: mol_type = genomic DNA
ggcagagggagccaagcaactaatcaaactcaaagcgcctcctaccaaccgaaaaggcgagaaaaagctaagccaaggtggggatcggaagaattatcca

SEQ ID NO: 286 - DNA, 100 bp, Saccharum spontaneum
  source 1..100: mol_type = genomic DNA
tagcaagtgcggctgctatcttgagaggttggcgctgtggtgctaggctttttgccgtgctgggtgcctgggtggtggagatggatggatgtatatatag

SEQ ID NO: 287 - DNA, 169 bp, Saccharum spontaneum
  source 1..169: mol_type = genomic DNA
gtatgtcacctttgagggttttgacttttaatgaatttgcaactatggcacccaatttttgcagctgtagtgttgcttccatcatcagtcaggtctacacctaccaaataattctagtgttccatggctccaatgaaatgtggattgaaagttaatcttaattgcatta

SEQ ID NO: 288 - DNA, 160 bp, Saccharum spontaneum
  source 1..160: mol_type = genomic DNA
gtgagcgcccagatgttcctgagacagcagctgagcaggacgctacagttgacgagcttgctgaggcagagctcgcagaccttgagactcagactgctgacatacctgaggatgtcacttcagatagcacagatgaggactaccagcccattcccaggta

SEQ ID NO: 289 - DNA, 160 bp, Saccharum spontaneum
  source 1..160: mol_type = genomic DNA
gtgagcgcccagatgttcctgagacagcagctgagcaggacgctacagttgacgagcttgctgaggcagagctcgcggatcttgaggctcagactgctgacatacctgaggatgccacttctgatagcacagatgaggactaccagcccattcccaggta

SEQ ID NO: 290 - DNA, 100 bp, Saccharum spontaneum
  source 1..100: mol_type = genomic DNA
gtggggcgtgtgtctcacccaacgaagtagtggccaagtaaggtagccagcggtgggcgagctccttatttgatgacgtggtccagaaaatggttctctt

SEQ ID NO: 291 - DNA, 100 bp, Saccharum spontaneum
  source 1..100: mol_type = genomic DNA
tcatgtccaagtactcgcaagctgatgcttgggggctacaaccactggggtctcctgagcgcaaattgtcaggatcgcgcgctgattctaccacgcggcc

SEQ ID NO: 292 - DNA, 146 bp, Saccharum spontaneum; identical to SEQ ID NO: 151
  source 1..146: mol_type = genomic DNA

SEQ ID NO: 293 - DNA, 148 bp, Saccharum spontaneum; identical to SEQ ID NO: 1
  source 1..148: mol_type = genomic DNA

SEQ ID NO: 294 - DNA, 56 bp, Saccharum spontaneum
  source 1..56: mol_type = genomic DNA
tcaaatgaaggccaagcgtcaagcagtgagcattctttcaatttcttggcaaccca

SEQ ID NO: 295 - DNA, 80 bp, Saccharum spontaneum
  source 1..80: mol_type = genomic DNA
caaagtctagtaggtactctcgagaccctcatacgctctgatggcggcgaacatagcactcattgcctggttgtcgctag

SEQ ID NO: 296 - DNA, 100 bp, Saccharum spontaneum
  source 1..100: mol_type = genomic DNA
caagcactgggttgattctggcaactacggcatcaaatttgttcctctgggaccgggcttcttgctccagcacatcgaactgtgccttgaccctctgatg

SEQ ID NO: 297 - DNA, 100 bp, Saccharum spontaneum
  source 1..100: mol_type = genomic DNA
gcgagcgaacaaactgaccgaggagcttaacggtaagtatccactggtcgggttttctgctgtagcgattgtcttgcctgatggaacttcaaaatgcatg

SEQ ID NO: 298 - DNA, 126 bp, Saccharum spontaneum; identical to SEQ ID NO: 248
  source 1..126: mol_type = genomic DNA

SEQ ID NO: 299 - DNA, 100 bp, Saccharum spontaneum
  source 1..100: mol_type = genomic DNA
ctaaaggaagaaaccaacacgacaaaaaacagaaaaggtacgtaccagcgtggaagttctcaagggtggagaattcctcggcctgccgagccacctgctc

SEQ ID NO: 300 - DNA, 100 bp, Saccharum spontaneum
  source 1..100: mol_type = genomic DNA
gcggagcaccttggtgtcggagcagaacgccttggagttggcttggaatgccctggagtcaaagcgaaatgcctggtcagaggcggaccaggaggtgctc

SEQ ID NO: 301 - DNA, 100 bp, Saccharum spontaneum
  source 1..100: mol_type = genomic DNA
ctcgggcggtgtcggagaggaacagggggagttgtcgaatgataacccggtcgtcggtggccccacccagctgacatgccaagcggaaatcggcaagcca

SEQ ID NO: 302 - DNA, 100 bp, Saccharum spontaneum
  source 1..100: mol_type = genomic DNA
tcgaaagatcgtagtccttcacccgagcctccgggccctcgggtcttcagtaaggctatccgtgaaacggtgctcccggctcggtttcgccctcccacga

SEQ ID NO: 303 - DNA, 170 bp, Saccharum spontaneum; identical to SEQ ID NO: 270
  source 1..170: mol_type = genomic DNA

SEQ ID NO: 304 - DNA, 100 bp, Saccharum spontaneum
  source 1..100: mol_type = genomic DNA
ctggaagggtgatttagtctctgatcggaaggcctagctaaggaggaacaacgctcgcttgcgactctgacctgcctctccgaccggaaggcctggccaa

SEQ ID NO: 305 - DNA, 100 bp, Saccharum spontaneum
  source 1..100: mol_type = genomic DNA
tcggagtcagaatcgagcgtcgttcctccttggctaggccttttggttggagaggcgggtcagagttagaagcgagcatcgttcctccttggccaggcct

SEQ ID NO: 306 - DNA, 85 bp, Saccharum spontaneum
  source 1..85: mol_type = genomic DNA
ctggcacctcctccaaactctttccttctctcctctctctatttctaaagactagatcctaataaggactaatcttctctcgata

SEQ ID NO: 307 - DNA, 84 bp, Saccharum spontaneum; identical to SEQ ID NO: 271
  source 1..84: mol_type = genomic DNA

SEQ ID NO: 308 - DNA, 100 bp, Saccharum spontaneum
  source 1..100: mol_type = genomic DNA
gaagggtggtaggtgggcatcgaacggacctggtacatgggcttgggaaagccatagaagtcgtagatggtgtcgttgctgccgcggataacgttgaccg

SEQ ID NO: 309 - DNA, 100 bp, Saccharum spontaneum
  source 1..100: mol_type = genomic DNA
gcgggcagacccaaaccagagacgcggcacgcgacgcgggagggcgaggagcagcggcggcgcggcgacatcgcgaggccagggccagacgcaacggccg

SEQ ID NO: 310 - DNA, 146 bp, Saccharum spontaneum
  source 1..146: mol_type = genomic DNA
gacacacgacacgagagtttggccatggcaacggtctcctcgctcgctcgctcctcactggcggccgtcacacctggagagtgaccgactggcagtagctgtagcagccctcagcctcacgcgacgcagcgcaggcctctggatgc

SEQ ID NO: 311 - DNA, 160 bp, Saccharum spontaneum; identical to SEQ ID NO: 273
  source 1..160: mol_type = genomic DNA

SEQ ID NO: 312 - DNA, 100 bp, Saccharum spontaneum
  source 1..100: mol_type = genomic DNA
gacgcggcgatcaggccggaaggagtcgtggcgccgccgtccgtgagcttacctgattgatttgattgattgttgcattggttaattattattagttact

SEQ ID NO: 313 - DNA, 100 bp, Saccharum spontaneum
  source 1..100: mol_type = genomic DNA
gagaatgccggtgagcaggatcgccgtgggcgctccgggcgagctgtcccaccccgacaccgccaaggccgccgtcgccgagttcatctccatgctcatc

SEQ ID NO: 314 - DNA, 111 bp, Saccharum spontaneum
  source 1..111: mol_type = genomic DNA
gagcggcggcgacggcggaaatggagagggcggtggggggaaagcggcctagggttcgaggggagagggaatggatacgattgaggggtgaggggcgcggggatggggcga

SEQ ID NO: 315 - DNA, 155 bp, Saccharum spontaneum
  source 1..155: mol_type = genomic DNA
gaggacagcgaccagagctggaggaggatcgatcggcggagggtggtctgttatgttccgtgaccaggcgtgactccgtgtcgcatatgattagggaaaggatatgaaccgtgagctccgtggcgctgaagttgcgccggttagctggtcactga

SEQ ID NO: 316 - DNA, 146 bp, Saccharum spontaneum; identical to SEQ ID NO: 151
  source 1..146: mol_type = genomic DNA

SEQ ID NO: 317 - DNA, 148 bp, Saccharum spontaneum
  source 1..148: mol_type = genomic DNA
tagcccactaaaataaaaccttgcatactccttggtgtcactttatttctgtttaaggcgggtaagtctagctgagtaccctctcgtactcagggctttgttcctattgttgttgcagatggccagatgtactatggttattgcatca

SEQ ID NO: 318 - DNA, 112 bp, Saccharum spontaneum
  source 1..112: mol_type = genomic DNA
tcggaacgtcaaggccaagtacatgagctctggagcactcagaagccagacatcgatcaactcatgttgagccatcagcgagccgtgtcgatccatcaaacaagccgtccga

(19) United States
(12) Patent Application Publication
(10) Pub. No.: US 2024/0043862 A1
(43) Pub. Date: Feb. 8, 2024

(54) MARKER ASSOCIATED WITH SMUT RESISTANCE IN PLANT BELONGING TO GENUS SACCHARUM AND USE THEREOF

(71) Applicant: TOYOTA JIDOSHA KABUSHIKI KAISHA, Aichi (JP)

(72) Inventors: Masaaki MORI, Okazaki shi (JP); Tatsuro KIMURA, Kariya-shi (JP); Hiroyuki ENOKI, Hamamatsu-shi (JP); Yusuke UETA, Miyoshi-shi (JP); Takeo SAKAIGAICHI, Miyakonojo-shi (JP); Yusuke TARUMOTO, Koshi-shi (JP); Minoru TANAKA, Tsukuba-shi (JP); Katsuki ADACHI, Nishinoomote-shi (JP); Taichiro HATTORI, Nishinoomote-shi (JP); Makoto UMEDA, Nishinoomote-shi (JP); Michiko HAYANO, Nishinoomote-shi (JP)

(73) Assignee: TOYOTA JIDOSHA KABUSHIKI KAISHA, Aichi (JP)

(21) Appl. No.: 18/478,428

(22) Filed: Sep. 29, 2023

Related U.S. Application Data
(62) Division of application No. 17/257,453, filed on Dec. 31, 2020, now Pat. No. 11,814,638.

(30) Foreign Application Priority Data
Jul. 3, 2018 (JP) 2018-127142
Oct. 19, 2018 (JP) 2018-197546
Jul. 1, 2019 (JP) 2019-122913

(51) Int. Cl. C12N 15/82; C12Q 1/6895; A01H 1/04; A01H 1/00

(52) U.S. Cl. CPC C12N 15/8282; C12Q 1/6895; A01H 1/045; A01H 1/1255; C12Q 2600/156

(57) ABSTRACT

A method for producing a sugarcane line having improved smut resistance, the method including: a step of breeding parent plants to obtain a progeny plant, wherein at least one of said parent plants is a sugarcane plant; a step of extracting a genomic DNA of the progeny plant; a step of determining the presence or absence of a marker associated with sugarcane smut resistance in the obtained genomic DNA; a step of selecting a progeny plant having the marker associated with sugarcane smut resistance, and a step of breeding only the selected progeny plant; wherein the marker associated with sugarcane smut resistance contains any nucleotide sequence selected from the group consisting of the nucleotide sequences of SEQ ID NOs: 144 to 151, or at least 15 consecutive nucleotides of SEQ ID NOs: 144 to 151.

### CROSS REFERENCE TO RELATED APPLICATIONS

The present application is a Divisional Application of U.S. patent application Ser. No. 17/257,453, filed Dec. 31, 2020, which is a 35 U.S.C. § 371 national stage patent application of International Patent Application PCT/JP2019/026324, filed Jul. 2, 2019, which claims priority to Japanese patent applications JP 2018-127142, filed Jul. 3, 2018, JP 2018-197546, filed Oct. 19, 2018 and JP 2019-122913, filed on Jul. 1, 2019. The disclosure of U.S. patent application Ser. No. 17/257,453 is incorporated herein by reference in its entirety.

### INCORPORATION BY REFERENCE OF SEQUENCE LISTING

The content of the electronically submitted sequence listing, file name: Q291585_sequence listing as filed.xml; size: 375 kb; and date of creation: Sep. 17, 2023, filed herewith, is incorporated herein by reference in their entirety.

### TECHNICAL FIELD

The present invention relates to a marker associated with smut resistance that enables selection of a sugarcane line resistant to smut and a method for using the same.

### BACKGROUND ART

Sugarcane has been cultivated for edible use such as a raw material for sugar, liquor, and the like. In addition, sugarcane has been used in a variety of industrial fields, including the use thereof as a raw material for biofuel. Under such circumstances, there is a need to develop novel sugarcane varieties having desirable characteristics (e.g., sugar content, enhanced vegetative capacity, sprouting capacity, disease resistance, insect resistance, cold resistance, an increase in leaf blade length, an increase in leaf area, and an increase in stalk length).

In general, there are three methods for identification of a plant variety/line: that is, “characteristics comparison” for comparison of characteristics data; “comparison during cultivation” for comparison of plants cultivated under the same conditions; and “DNA assay” for DNA analysis. There are many problems in line identification by means of characteristics comparison or comparison during cultivation, such as lowered precision due to differences in cultivation conditions and long-term field research that requires a number of steps. In particular, sugarcane plants are much larger than other graminaceous crops such as rice and maize, and it is thus difficult to conduct line identification through field research.

In order to identify a variety resistant to a certain disease, in addition, it is necessary to carry out an inoculation test using a causative microorganism of a disease after long-term cultivation of sugarcane and then collect disease resistance data by observing lesions and the like. When the test is carried out, however, it is necessary to prevent the causative microorganism from propagation to an external environment more definitely and provide facilities, such as a large-scale special-purpose greenhouse, a special-purpose field, or a facility isolated from an external environment. In order to prepare a novel sugarcane variety, in addition, tens of thousands of hybrids are first prepared via crossing, followed by seedling selection and stepwise selection of elite lines. Eventually, 2 or 3 types of candidate novel varieties having desirable characteristics can be obtained. For preparation of a novel sugarcane variety, as described above, it is necessary to cultivate and evaluate an enormous number of lines, and it is also necessary to prepare the large-scale greenhouse or field as described above and make time-consuming efforts.

Therefore, it has been required to develop a method for identification of a sugarcane line having disease resistance with the use of markers present in the sugarcane genome. If markers excellent in a variety of characteristics could be used for production of a novel sugarcane variety, in particular, various problems peculiar to sugarcane as described above would be resolved, and the markers would be able to serve as very effective tools. However, sugarcane plants have a large number of chromosomes (approximately 100 to 130) due to higher polyploidy, and the development of marker technology has been thus slow. In the case of sugarcane, the USDA reported genotyping with the use of SSR markers (Non-Patent Literature 1), although precision of genotyping is low because of the small numbers of markers and polymorphisms in each marker. In addition, the above genotyping is available only for American/Australian varieties, and it cannot be used for identification of major varieties cultivated in Japan, Taiwan, India, and other countries and lines serving as useful genetic resources.

In addition, Non-Patent Literature 2 suggests the possibility that a sugarcane genetic map can be prepared by increasing the number of markers, comparing individual markers in terms of a characteristic relation, and verifying the results. However, Non-Patent Literature 2 does not disclose a sufficient number of markers, and markers linked to characteristics of interest have not been found.

As disclosed in Patent Literature 1, a marker associated with black root rot resistance in sugar beet is known as a marker associated with disease resistance. Patent Literature 2 also discloses a technique of selecting a zea mays variety using a maker linked to a trait of interest. Meanwhile, causative microorganisms of sugarcane smut have high-level infectivity, and, therefore, the onset of smut results in immediate infection of the entire field. Sugarcane crops affected with smut cannot be used as raw material for sugar production, and, in addition, such affected crops would wither and die. Therefore, development of smut will cause a significant decline in yield in the following year and later. Damage due to smut has been reported in 28 or more countries, including Brazil, U.S.A., Australia, China, and Indonesia. Smut can be prevented by sterilization treatment at the time of planting; however, preventive effects are limited to the early growth period. Thus, cultivation of a sugarcane variety imparted with smut resistance has been awaited.

Patent Literature 3 discloses markers linked to smut resistance that were discovered by preparing many sugarcane plant markers and conducting linkage analysis of quantitative traits along with such markers for hybrid progeny lines.

### CITATION LIST

### Non Patent Literature

Non Patent Literature 1: Maydica 48, 2003, 319-329, “Molecular genotyping of sugarcane clones with microsatellite DNA markers”

Non Patent Literature 2: Nathalie Piperidis et al., Molecular Breeding, 2008, Vol. 21, 233-247

### Patent Literature

Patent Literature 1: WO 2007/125958

Patent Literature 2: JP 2010-516236 A

Patent Literature 3: WO 2012/147635

### SUMMARY OF INVENTION

### Technical Problem

While Patent Literature 3 discloses markers linked to smut resistance, development of smut resistance markers exhibiting higher association with smut resistance had been awaited. Under the above circumstances, the present invention is intended to provide an improved smut resistance marker exhibiting higher association with smut resistance.

### Solution to Problem

The present inventors have conducted intensive studies to achieve the object. The present inventors prepared many markers of particular sugarcane varieties and conducted linkage analysis of quantitative traits along with such markers for hybrid progeny lines. Accordingly, the present inventors found markers linked to quantitative traits such as smut resistance. This has led to the completion of the present invention.

The present invention encompasses the following.

(1) A marker associated with sugarcane smut resistance, which consists of a continuous nucleic acid region existing in a region between the nucleotide sequence as shown in SEQ ID NO: 1 and the nucleotide sequence as shown in SEQ ID NO: 6, a region between the nucleotide sequence as shown in SEQ ID NO: 135 and the nucleotide sequence as shown in SEQ ID NO: 143, or a region between the nucleotide sequence as shown in SEQ ID NO: 144 or 145 and the nucleotide sequence as shown in SEQ ID NO: 151 of a sugarcane chromosome.

(2) The marker associated with sugarcane smut resistance according to (1), wherein the nucleic acid region comprises any nucleotide sequence selected from the group consisting of the nucleotide sequences as shown in SEQ ID NOs: 1 to 6, 135 to 143, and 144 to 151 or a part of the nucleotide sequence.

(3) The marker associated with sugarcane smut resistance according to (1), wherein the nucleic acid region comprises the nucleotide sequence as shown in SEQ ID NO: 1 or 2 of a sugarcane chromosome or a part of the nucleotide sequence.

(4) The marker associated with sugarcane smut resistance according to (1), wherein the nucleic acid region comprises any nucleotide sequence selected from the group consisting of the nucleotide sequences as shown in SEQ ID NOs: 138 to 140 of a sugarcane chromosome or a part of the nucleotide sequence.

(5) The marker associated with sugarcane smut resistance according to (1), wherein the nucleic acid region comprises a nucleotide sequence as shown in SEQ ID NO: 149 or 151 of a sugarcane chromosome or a part of the nucleotide sequence.

(6) The marker associated with sugarcane smut resistance according to (1), wherein the nucleic acid region comprises any nucleotide sequence selected from the group consisting of the nucleotide sequences as shown in SEQ ID NOs: 298, 303, 307, 311, and 316 of a sugarcane chromosome or a part of the nucleotide sequence.

(7) A method for producing a sugarcane line having improved smut resistance comprising: a step of extracting a chromosome of a progeny plant obtained from parent plants, at least one of which is a sugarcane plant, and/or a chromosome of a parent sugarcane plant; and a step of determining the presence or absence of the marker associated with sugarcane smut resistance according to any one of (1) to (6) in the obtained chromosome.

(8) The method for producing a sugarcane line according to (7), wherein the step of determination involves the use of a DNA chip comprising a probe corresponding to the marker associated with sugarcane smut resistance.

(9) The method for producing a sugarcane line according to (7), wherein the progeny plant is a seed or young seedling and the chromosome is extracted from the seed or young seedling.

This description includes part or all of the content as disclosed in the descriptions and/or drawings of Japanese Patent Application Nos. 2018-127142, 2018-197546, and 2019-122913, which are priority documents of the present application.

### Advantageous Effects of Invention

The present invention can provide a novel marker associated with sugarcane smut resistance that is linked particularly to smut resistance among various sugarcane quantitative traits. With the use of the marker associated with sugarcane smut resistance of the present invention, smut resistance of hybrid sugarcane progeny lines can be tested. Thus, a sugarcane variety with improved smut resistance can be identified in a very cost-effective manner.

### BRIEF DESCRIPTION OF DRAWINGS

FIGS. 1 (A)-1 (C) show characteristic diagrams showing: FIG. 1 (A) the results of calculation of the morbidity of smut of a progeny line resulting from crossbreeding between “KY08-6023” and “JW90”; FIG. 1 (B) the results of calculation of the morbidity of smut of a progeny line resulting from crossbreeding between “KY08-6039” and “JW90”; and FIG. 1 (C) the results of calculation of the morbidity of smut of a progeny line resulting from crossbreeding between “KY08-6041” and “JW90”.

FIG. 2 shows a characteristic diagram showing the results of QTL analysis concerning smut resistance conducted in Example 1.

FIG. 3 shows a characteristic diagram showing the number of reads of AMP0121265 in each line.

FIG. 4 shows a characteristic diagram showing the number of reads of AMP0120752 in each line.

FIG. 5 shows a characteristic diagram showing the number of reads of AMP0035185 in each line.

FIG. 6 shows a characteristic diagram showing the number of reads of AMP0114852 in each line.

FIG. 7 shows a characteristic diagram showing the number of reads of AMP0089904 in each line.

FIG. 8 shows a characteristic diagram showing the number of reads of AMP0100370 in each line.

FIG. 9 shows a characteristic diagram showing the results of QTL analysis concerning smut resistance conducted in Example 2.

FIG. 10 shows a characteristic diagram showing the number of reads of AMP0014532 in each line.

FIG. 11 shows a characteristic diagram showing the number of reads of AMP0043152 in each line.

FIG. 12 shows a characteristic diagram showing the number of reads of AMP0069135 in each line.

FIG. 13 shows a characteristic diagram showing the number of reads of AMP0032477 in each line.

FIG. 14 shows a characteristic diagram showing the number of reads of AMP0018405 in each line.

FIG. 15 shows a characteristic diagram showing the number of reads of AMP0002312 in each line.

FIG. 16 shows a characteristic diagram showing the number of reads of AMP0007121 in each line.

FIG. 17 shows a characteristic diagram showing the number of reads of AMP0090108 in each line.

FIG. 18 shows a characteristic diagram showing the number of reads of AMP0015886 in each line.

FIG. 19 shows a characteristic diagram showing the results of calculation of the morbidity of smut for progeny lines resulting from crossing between “KY09-6092” and “KY08-129.”

FIG. 20 shows a characteristic diagram showing the results of QTL analysis of smut resistance performed in Example 3.

FIG. 21 shows a characteristic diagram showing the number of reads of AMP0063683 in each line.

FIG. 22 shows a characteristic diagram showing the number of reads of AMP0082090 in each line.

FIG. 23 shows a characteristic diagram showing the number of reads of AMP0013802 in each line.

FIG. 24 shows a characteristic diagram showing the number of reads of AMP0083204 in each line.

FIG. 25 shows a characteristic diagram showing the number of reads of AMP0043774 in each line.

FIG. 26 shows a characteristic diagram showing the number of reads of AMP0094596 in each line.

FIG. 27 shows a characteristic diagram showing the number of reads of AMP0091501 in each line.

### DESCRIPTION OF EMBODIMENTS

The marker associated with sugarcane smut resistance according to the present invention and the method for using the same are described below. In particular, a method for producing a sugarcane line using a marker associated with sugarcane smut resistance is described.

[Markers Associated with Sugarcane Smut Resistance]

The marker associated with sugarcane smut resistance of the present invention is a particular region present on a sugarcane chromosome, it is linked to a causative gene (or a group of causative genes) for a trait of sugarcane smut resistance, and it can be thus used for identification of a trait of sugarcane smut resistance. Therefore, whether or not a progeny line obtained with the use of a known sugarcane line has a trait of improving smut resistance can be determined by confirming the presence or absence of the marker associated with sugarcane smut resistance in such progeny line. In the present invention, the term “smut” refers to a disease characterized by lesion formation due to infection with a microorganism of the genus Ustilago. One example of a microorganism of the genus Ustilago is Ustilago scitaminea.

In addition, the term “marker associated with sugarcane smut resistance” refers to a marker linked to a causal gene (or a group of causative genes) of a trait of improving smut resistance. In the presence of the marker of interest in a certain sugarcane variety, for example, such sugarcane variety can be determined to have improved smut resistance.

The term “sugarcane” used herein refers to a plant belonging to the genus Saccharum of the family Poaceae. In addition, the term “sugarcane” includes so-called noble cane (scientific name: Saccharum officinarum) and wild cane (scientific name: Saccharum spontaneum), Saccharum barberi, Saccharum sinense, and the earlier species of Saccharum officinarum (Saccharum robustum). The term “known sugarcane variety/line” is not particularly limited. It includes any variety/line usable in Japan and any variety/line used outside Japan. Examples of sugarcane varieties cultivated in Japan include, but are not particularly limited to, Ni1, NiN2, NiF3, NiF4, NiF5, Ni6, NiN7, NiF8, Ni9, NiTn10, Ni11, Ni12, Ni14, Ni15, Ni16, Ni17, NiTn19, NiTn20, Ni22, and Ni23. Examples of major sugarcane varieties used in Japan include, but are not particularly limited to, NiF8, Ni9, NiTn10, and Ni15. Further, examples of major sugarcane varieties that have been introduced into Japan include, but are not limited to, F177, Nco310, and F172. In addition, sugarcane varieties and lines are wild-type varieties with excellent disease resistance, and wild-type varieties with excellent smut resistance are used. Examples of wild-type varieties with excellent smut resistance include, but are not particularly limited to, JW90, Iriomote 15, and Iriomote 8.

In addition, a progeny line may be a line obtained by crossing a maternal plant and a paternal plant of the same species, each of which is a sugarcane variety/line, or it may be a hybrid line obtained from parent plants when one thereof is a sugarcane variety/line and the other is a closely related variety/line (Erianthus arundinaceus). In addition, a progeny line may be obtained by so-called backcrossing. In particular, both or either the maternal line and the paternal line is preferably a wild-type variety with excellent smut resistance, such as JW90, Iriomote 15, or Iriomote 8.

### Example of Markers Associated with Sugarcane Smut Resistance 1

The marker associated with sugarcane smut resistance was newly identified by quantitative trait loci (QTL) analysis using a genetic linkage map comprising 86 linkage groups including 31,191 markers (4,503 thereof are derived from JW90) originally obtained from sugarcane chromosomes and the sugarcane smut resistance data. In addition, many genes are presumably associated with sugarcane smut resistance, which is a quantitative trait characterized by a continuous distribution. That is, sugarcane smut resistance is evaluated based on the morbidity of smut characterized by such continuous distribution. For QTL analysis, the QTL Cartographer gene analysis software (Wang S., C. J. Basten, and Z. B. Zeng, 2010; Windows QTL Cartographer 2.5, Department of Statistics, North Carolina State University, Raleigh, N.C.) is used, and the analysis is carried out by the composite interval mapping (CIM) method.

Specifically, a region included in the above genetic linkage map with LOD scores equivalent to or exceeding a given threshold (e.g., 2.5); i.e., a region of approximately 8.4 cM (centimorgan), was identified by the QTL analysis described above. The term “morgan (M)” used herein refers to a unit representing the relative distance between genes on a chromosome, and it expresses the crossover rate in percent figures. In the case of a sugarcane chromosome, 1 cM corresponds to approximately 2,000 kb. In addition, it is suggested that a causative gene (or a group of causative genes) for a trait that improves smut resistance could be present at the peak position or in the vicinity thereof.

The 8.4 cM region comprises 6 types of markers listed in Table 1 in the order shown therein and it is linked to a trait of improving smut resistance.

TABLE 1

| | Linkage | Position | | Adjacent | LOD | Effects |
|---|---|---|---|---|---|---|
| Varity | group | (cM) | Range (cM) | marker | value | (%) |
| JW90 | 42 | 0.0 | 8.4 | AMP0121265 | 24.5 | −47.2 |
| | | | | (SEQ ID NO: 1)- | | |
| | | | | AMP0120752 | | |
| | | | | (SEQ ID NO: 2)- | | |
| | | | | AMP0035185 | | |
| | | | | (SEQ ID NO: 3)- | | |
| | | | | AMP0114852 | | |
| | | | | (SEQ ID NO: 4)- | | |
| | | | | AMP0089904 | | |
| | | | | (SEQ ID NO: 5)- | | |
| | | | | AMP0100370 | | |
| | | | | (SEQ ID NO: 6) | | |

In Table 1, “Linkage group” represents the number given to each group among a plurality of linkage groups specified by QTL analysis. In Table 1, the name of the marker provided in the column indicating adjacent markers represents the name given to each marker originally obtained in the present invention.

In addition, the peak included in the 8.4 cM region is present adjacent to the marker comprising the nucleotide sequence as shown in SEQ ID NO: 1 (AMP0121265).

A continuous nucleic acid region existing in the 8.4 cM region containing markers shown in Table 1 can be used as a marker associated with sugarcane smut resistance. The term “nucleic acid region” used herein refers to a region comprising a nucleotide sequence having 95% or less, preferably 90% or less, more preferably 80% or less, and most preferably 70% or less identity to a different region present on a sugarcane chromosome. If the identity of a nucleic acid region serving as a marker associated with sugarcane smut resistance to a different region falls within the above range, the nucleic acid region can be specifically detected in accordance with a conventional technique. The identity value described herein can be calculated using, for example, BLAST® with default parameter settings.

The nucleic acid region serving as a marker associated with sugarcane smut resistance can comprise 8 or more, preferably 15 or more, more preferably 20 or more, and most preferably nucleotides. If the length of the nucleic acid region serving as a marker associated with sugarcane smut resistance falls within the above range, the nucleic acid region can be specifically detected in accordance with a conventional technique.

The marker associated with sugarcane smut resistance may be any continuous nucleic acid region selected from the 8.4 cM region. The nucleotide sequence of the 8.4 cM region can be identified by flanking sequence analysis such as inverse PCR analysis using primers designed based on the nucleotide sequences as shown in SEQ ID NOs: 1 to 6.

In particular, the marker associated with sugarcane smut resistance is preferably selected from a region adjacent to the nucleotide sequence as shown in SEQ ID NO: 1 or 2 in the 8.4 cM region described above because of the presence of the peak adjacent to the nucleotide sequence as shown in SEQ ID NO: 1.

The 6 types of the markers or some thereof can be used as the marker associated with sugarcane smut resistance. Specifically, one or more markers selected from among the 6 types of markers can be used as the markers associated with sugarcane smut resistance. Alternatively, a partial region of the 6 types of markers can be used as the marker associated with sugarcane smut resistance.

### Example of the Marker Associated with Sugarcane Smut Resistance 2

The marker associated with sugarcane smut resistance was newly identified by quantitative trait loci (QTL) analysis using a genetic linkage map of progeny plants of Iriomote 15 comprising 58 linkage groups including 64,757 markers (1,166 thereof are derived from progeny plants of Iriomote 15) originally obtained from sugarcane chromosomes and the sugarcane smut resistance data. In addition, many genes are presumably associated with sugarcane smut resistance, which is a quantitative trait characterized by a continuous distribution. That is, sugarcane smut resistance is evaluated based on the morbidity of smut characterized by such continuous distribution. For QTL analysis, the QTL Cartographer gene analysis software (Wang S., C. J. Basten, and Z.-B. Zeng, 2010; Windows QTL Cartographer 2.5, Department of Statistics, North Carolina State University, Raleigh, N.C.) is used, and the analysis is carried out by the composite interval mapping (CIM) method.

Specifically, a region included in the above genetic linkage map with LOD scores equivalent to or exceeding a given threshold (e.g., 2.5); i.e., a region of approximately 26.6 cM (centimorgan), was identified by the QTL analysis described above. The term “morgan (M)” used herein refers to a unit representing the relative distance between genes on a chromosome, and it expresses the crossover rate in percent figures. In the case of a sugarcane chromosome, 1 cM corresponds to approximately 2,000 kb. In addition, it is suggested that a causative gene (or a group of causative genes) for a trait that improves smut resistance could be present at the peak position or in the vicinity thereof.

The 26.6 cM region comprises 9 types of markers listed in Table 2 in the order shown therein and it is linked to a trait of improving smut resistance.

TABLE 2

| | Linkage | Position | Range | Adjacent | LOD | Effects |
|---|---|---|---|---|---|---|
| Vanty | group | (cM) | (cM) | marker | value | (%) |
| Iriomote 15 | 15 | 25.7 | 26.6 | AMP0014532 | 11.5 | −37.8 |
| | | | | (SEQ ID NO: 135)- | | |
| | | | | AMP0043152 | | |
| | | | | (SEQ ID NO: 136)- | | |
| | | | | AMP0069135 | | |
| | | | | (SEQ ID NO: 137)- | | |
| | | | | AMP0032477 | | |
| | | | | (SEQ ID NO: 138)- | | |
| | | | | AMP0018405 | | |
| | | | | (SEQ ID NO: 139)- | | |
| | | | | AMP0002312 | | |
| | | | | (SEQ ID NO: 140)- | | |
| | | | | AMP0007121 | | |
| | | | | (SEQ ID NO: 141)- | | |
| | | | | AMP0090108 | | |
| | | | | (SEQ ID NO: 142)- | | |
| | | | | AMP0015886 | | |
| | | | | (SEQ ID NO: 143) | | |

In Table 2, “Linkage group” represents the number given to each group among a plurality of linkage groups specified by QTL analysis. In Table 2, the name of the marker provided in the column indicating adjacent markers represents the name given to each marker originally obtained in the present invention.

In addition, the peak included in the 26.6 cM region is present in a position between the marker comprising the nucleotide sequence as shown in SEQ ID NO: 138 (AMP0032477) and the marker comprising the nucleotide sequence as shown in SEQ ID NO: 140 (AMP002312). In particular, the peak is present adjacent to the marker comprising the nucleotide sequence as shown in SEQ ID NO: 139 (AMP0018405).

A continuous nucleic acid region existing in the 26.6 cM region containing markers shown in Table 2 can be used as a marker associated with sugarcane smut resistance. The term “nucleic acid region” used herein refers to a region comprising a nucleotide sequence having 95% or less, preferably 90% or less, more preferably 80% or less, and most preferably 70% or less identity to a different region present on a sugarcane chromosome. If the identity of a nucleic acid region serving as a marker associated with sugarcane smut resistance to a different region falls within the above range, the nucleic acid region can be specifically detected in accordance with a conventional technique. The identity value described herein can be calculated using, for example, BLAST® with default parameter settings.

The nucleic acid region serving as a marker associated with sugarcane smut resistance can comprise 8 or more, preferably 15 or more, more preferably 20 or more, and most preferably nucleotides. If the length of the nucleic acid region serving as a marker associated with sugarcane smut resistance falls within the above range, the nucleic acid region can be specifically detected in accordance with a conventional technique.

The marker associated with sugarcane smut resistance may be any continuous nucleic acid region selected from the 26.6 cM region. The nucleotide sequence of the 26.6 cM region can be identified by flanking sequence analysis such as inverse PCR analysis using primers designed based on the nucleotide sequences as shown in SEQ ID NOs: 135 to 143.

In particular, the marker associated with sugarcane smut resistance is preferably selected from the region between the nucleotide sequence as shown in SEQ ID NO: 138 and the nucleotide sequence as shown in SEQ ID NO: 140 in the 26.6 cM region described above. In addition, the marker associated with sugarcane smut resistance is preferably selected from the region comprising the nucleotide sequence as shown in SEQ ID NO: 139 and a nucleotide sequence adjacent to the nucleotide sequence as shown in SEQ ID NO: 139 in the 26.6 cM region described above. The marker is preferably selected in such manner because of the presence of the peak in the region between the nucleotide sequence as shown in SEQ ID NO: 138 and the nucleotide sequence as shown in SEQ ID NO: 140, which is adjacent to the nucleotide sequence as shown in SEQ ID NO: 139.

The 9 types of the markers or some thereof can be used as the markers associated with sugarcane smut resistance. Specifically, one or more markers selected from among the 9 types of markers can be used as the markers associated with sugarcane smut resistance. Alternatively, a partial region of the 9 types of markers can be used as the marker associated with sugarcane smut resistance.

### Example of Markers Associated with Sugarcane Smut Resistance 3

The marker associated with sugarcane smut resistance was newly identified by quantitative trait loci (QTL) analysis using a genetic linkage map derived from the progeny line “KY09-6092” of Iriomote 8 comprising 117 linkage groups including 57,444 markers (2,936 thereof are derived from the progeny line “KY09-6092” of Iriomote 8) originally obtained from sugarcane chromosomes and the sugarcane smut resistance data.

In addition, many genes are presumably associated with sugarcane smut resistance, which is a quantitative trait characterized by a continuous distribution. That is, sugarcane smut resistance is evaluated based on the morbidity of smut characterized by such continuous distribution. For QTL analysis, the QTL Cartographer gene analysis software (Wang S., C. J. Basten, and Z.-B. Zeng, 2010; Windows QTL Cartographer 2.5, Department of Statistics, North Carolina State University, Raleigh, N.C.) is used, and the analysis is carried out by the composite interval mapping (CIM) method.

Specifically, a region included in the above genetic linkage map with LOD scores equivalent to or exceeding a given threshold (e.g., 2.5); i.e., a region of approximately 12.27 cM (centimorgan), was identified by the QTL analysis described above. The term “morgan (M)” used herein refers to a unit representing the relative distance between genes on a chromosome, and it expresses the crossover rate in percent figures. In the case of a sugarcane chromosome, 1 cM corresponds to approximately 2,000 kb. In addition, it is suggested that a causative gene (or a group of causative genes) for a trait that improves smut resistance could be present at the peak position or in the vicinity thereof.

The 12.27 cM region comprises 7 types of markers listed in Table 3 in the order shown therein and it is linked to a trait of improving smut resistance.

TABLE 3

| | Linkage | Position | Range | Adjacent | LOD | Effects |
|---|---|---|---|---|---|---|
| | group | (cM) | (cM) | marker | value | (%) |
| Iriomote 8 | 8 | 83.8 | 12.27 | AMP0063683 | 68.7 | −54.2 |
| | | | | (SEQ ID NOs: 144, | | |
| | | | | 145)- | | |
| | | | | AMP0082090 | | |
| | | | | (SEQ ID NO: 146)- | | |
| | | | | AMP0013802 | | |
| | | | | (SEQ ID NO: 147)- | | |
| | | | | AMP0083204 | | |
| | | | | (SEQ ID NO: 148)- | | |
| | | | | AMP0043774 | | |
| | | | | (SEQ ID NO: 149)- | | |
| | | | | AMP0094596 | | |
| | | | | (SEQ ID NO: 150)- | | |
| | | | | AMP0091501 | | |
| | | | | (SEQ ID NO: 151) | | |

In Table 3, “Linkage group” represents the number given to each group among a plurality of linkage groups specified by QTL analysis. In Table 3, the name of the marker provided in the column indicating adjacent markers represents the name given to each marker originally obtained in the present invention. Among the markers shown in Table 3, AMP0063683 is a nucleic acid region comprising the nucleotide sequence as shown in SEQ ID NO: 144 and the nucleotide sequence as shown in SEQ ID NO: 145 at both ends. Markers other than AMP0063683 are each a nucleic acid region comprising a single nucleotide sequence.

In addition, the peak included in the 12.27 cM region is present adjacent to the marker comprising the nucleotide sequence as shown in SEQ ID NO: 151 (AMP0091501).

A continuous nucleic acid region existing in the 12.27 cM region containing markers shown in Table 3 can be used as a marker associated with sugarcane smut resistance. The term “nucleic acid region” used herein refers to a region comprising a nucleotide sequence having 95% or less, preferably 90% or less, more preferably 80% or less, and most preferably 70% or less identity to a different region present on a sugarcane chromosome. If the identity of a nucleic acid region serving as a marker associated with sugarcane smut resistance to a different region falls within the above range, the nucleic acid region can be specifically detected in accordance with a conventional technique. The identity value described herein can be calculated using, for example, BLAST® with default parameter settings.

The nucleic acid region serving as a marker associated with sugarcane smut resistance can comprise 8 or more, preferably 15 or more, more preferably 20 or more, and most preferably 30 nucleotides. If the length of the nucleic acid region serving as a marker associated with sugarcane smut resistance falls within the above range, the nucleic acid region can be specifically detected in accordance with a conventional technique.

The marker associated with sugarcane smut resistance may be any continuous nucleic acid region selected from the 12.27 cM region. The nucleotide sequence of the 12.27 cM region can be identified by flanking sequence analysis such as inverse PCR analysis using primers designed based on the nucleotide sequences as shown in SEQ ID NOs: 144 to 151.

In particular, the marker associated with sugarcane smut resistance is preferably selected from a region between the nucleotide sequence as shown in SEQ ID NO: 150 and the nucleotide sequence as shown in SEQ ID NO: 151 in the 12.27 cM region described above because of the presence of the peak adjacent to the nucleotide sequence as shown in SEQ ID NO: 151.

The 7 types of the markers or some thereof can be used as the marker associated with sugarcane smut resistance. Specifically, one or more markers selected from among the 7 types of markers can be used as the markers associated with sugarcane smut resistance. Alternatively, a partial region of the 7 types of markers can be used as the marker associated with sugarcane smut resistance.

A part of the 12.27 cM region comprising the markers shown in Table 3 comprises a partial region in common with a part of the 8.4 cM region comprising the markers shown in Table 1. Specifically, the nucleotide sequence of a region in the vicinity of the peak included in the 12.27 cM region is in common with the nucleotide sequence of a region in the vicinity of the peak included in the 8.4 cM region. More specifically, the nucleotide sequence of an adjacent region comprising AMP0121265 located at 0 cM among the markers associated with sugarcane smut resistance derived from JW90 shown in Table 1 is in common with the nucleotide sequence of an adjacent region comprising AMP0091501 located at 83.76 cM among the markers associated with sugarcane smut resistance derived from Iriomote 8 shown in Table 3. Accordingly, it is highly likely that the regions comprising the common nucleotide sequence comprises a factor (e.g., a causative gene) that improves sugarcane smut resistance. It is more preferable that such regions be used as the markers associated with sugarcane smut resistance.

[Sugarcane Marker Identification]

As described above, markers associated with sugarcane smut resistance were identified from among 31,191 markers originally obtained from sugarcane chromosomes (4,503 markers thereof are derived from JW90), 64,757 markers originally obtained from sugarcane chromosomes (1,166 markers thereof are derived from the progeny lines “KY08-6023,” “KY08-6039,” and “KY08-6041” of Iriomote 15), and 57,444 markers originally obtained from sugarcane chromosomes (2,936 markers thereof are derived from the progeny line “KY09-6092” of Iriomote 8) in the present invention. The 31,191 markers (4,503 markers thereof are derived from JW90), the 64,757 markers (1,166 markers thereof are derived from the progeny lines “KY08-6023,” “KY08-6039,” and “KY08-6041” of Iriomote 15), and the 57,444 markers (2,936 markers thereof are derived from the progeny line “KY09-6092” of Iriomote 8) are described herein. When identifying these markers, a DNA library was prepared in accordance with the method for preparing a DNA library disclosed in WO 2018/003220.

Specifically, a nucleic acid amplification reaction is carried out in a reaction solution in which a primer comprising an arbitrary nucleotide sequence (hereafter, referred to as a “random primer”) is adjusted at high concentration, and the amplified nucleic acid fragments are served as a DNA library. Here, the term “high concentration” refers to higher concentration than the primer concentration in a conventional nucleic acid amplification reaction. Specifically, the method for preparing a DNA library according to the present invention involves the use of a random primer at higher concentration than the primer concentration in a conventional nucleic acid amplification reaction. As a template included in the reaction solution, genomic DNA prepared from an organism, the DNA library of which is to be prepared, can be used.

The sequence of the random primer is not particularly limited. For example, a nucleotide comprising 9 to 30 bases can be used. In particular, a random primer is a nucleotide comprising an arbitrary sequence of 9 to 30 bases. A nucleotide type (a sequence type) is not particularly limited, and 1 or more types of nucleotides, preferably 1 to 10,000 types of nucleotides, more preferably 1 to 1,000 types of nucleotides, further preferably 1 to 100 types of nucleotides, and most preferably 1 to 96 types of nucleotides may be used. With the use of nucleotides (a group of nucleotides) within the range described above as a random primer, amplified nucleic acid fragments can be obtained with higher reproducibility. When a random primer comprises a plurality of nucleotides, it is not necessary that all nucleotides comprise the same number of bases (9 to 30 bases), and a random primer may comprise a plurality of nucleotides of a different number of bases.

In order to obtain a particular amplicon via a nucleic acid amplification reaction, in general, nucleotide sequences of primers are designed in accordance with the amplicon of interest. For example, a pair of primers is designed to flank the position corresponding to the amplicon in template DNA, such as genomic DNA. In such a case, primers are designed to hybridize to a specific region in the template, and it can thus be referred to as “specific primers.”

Unlike a primer designed to obtain a particular amplicon, in contrast, a random primer is not designed to hybridize to a specific region in template DNA, but it is designed to obtain a random amplicon. A random primer may comprise any nucleotide sequence. It accidentally hybridizes to a complementary region in template DNA and it can thus be involved in random amplicon amplification.

Specifically, a random primer can be a nucleotide comprising an arbitrary sequence involved in random amplicon amplification, as described above. An arbitrary sequence is not particularly limited. For example, it may be designed as a nucleotide sequence of nucleotides randomly selected from the group consisting of adenine, guanine, cytosine, and thymine, or it may be designed as a particular nucleotide sequence. Examples of particular nucleotide sequences include a nucleotide sequence comprising a restriction enzyme recognition sequence or a nucleotide sequence comprising an adaptor sequence applied to a next-generation sequencer.

When a plurality of types of nucleotides are designed as random primers, a plurality of nucleotide sequences of given length can be designed by randomly selecting nucleotides from the group consisting of adenine, guanine, cytosine, and thymine. When a plurality of types of nucleotides are designed as random primers, alternatively, a plurality of nucleotide sequences each comprising a common region composed of a particular nucleotide sequence and an uncommon region composed of an arbitrary nucleotide sequence can be designed. An uncommon region may be composed of a nucleotide sequence of nucleotides randomly selected from the group consisting of adenine, guanine, cytosine, and thymine. Alternatively, an uncommon region may comprise all of the 4 types of nucleotides; i.e., adenine, guanine, cytosine, and thymine, or some thereof. A common region is not particularly limited, and it may be composed of any nucleotide sequence. For example, a common region can be a nucleotide sequence comprising a restriction enzyme recognition sequence, a nucleotide sequence comprising an adaptor sequence applied to a next-generation sequencer, or a nucleotide sequence that is common among a particular gene family.

When a plurality of nucleotide sequences of given length are designed by randomly selecting nucleotides from among 4 types of nucleotides as a plurality of random primers, a plurality of nucleotide sequences are preferably designed to have 70% or lower, preferably 60% or lower, more preferably 50% or lower, and most preferably 40% or lower identity in regions constituting 30% or more, preferably 50% or more, more preferably 70% or more, and further preferably 90% or more of the entire sequences. When a plurality of nucleotide sequences of given length are designed by randomly selecting nucleotides from among 4 types of nucleotides as a plurality of random primers, the nucleotide sequences are designed to comprise the nucleotides having the identity within the range described above. Thus, amplified fragments can be obtained over the entire genomic DNA of the target organism species. That is, homogeneity of amplified fragments can be enhanced.

When a plurality of nucleotide sequences each comprising a common region composed of a particular nucleotide sequence and an uncommon region composed of an arbitrary nucleotide sequence are designed as a plurality of random primers, for example, several 3′-terminal nucleotides can be designed as an uncommon region, and remaining 5′-terminal nucleotides can be designed as a common region. If the “n” number of 3′-terminal nucleotides is designated as an uncommon region, 4ⁿ types of random primers can be designed. The number “n” can be 1 to 5, preferably 2 to 4, and more preferably 2 or 3.

As random primers each comprising a common region and an uncommon region, for example, 16 types of random primers each comprising a 5′-terminal adaptor sequence applied to a next-generation sequencer (a common region) and a 3′-terminal region of 2 nucleotides (an uncommon region) can be designed in total. When a 3′-terminal region is designed to comprise 3 nucleotides (an uncommon region), 64 types of random primers can be designed in total. As the types of random primers are increased, amplified fragments can be more extensively obtained throughout the entire genomic DNA of the target organism species. When designing a random primer comprising a common region and an uncommon region, accordingly, it is preferable that a 3′-terminal region comprise 3 nucleotides.

Alternatively, 64 types of nucleotide sequences each comprising a common region and an uncommon region of 3 nucleotides may be first designed, and up to 63 types of random primers selected from among the 64 types of nucleotide sequences may then be used. In other words, use of up to 63 types of random primers can occasionally yield results of analysis conducted by a nucleic acid amplification reaction or with the use of a next-generation sequencer superior to those attained with the use of all of 64 types of random primers.

It is preferable that random primer concentration be adequately determined in accordance with a base length of the random primer. When a plurality of types of nucleotides with different base length are used as random primers, the base length of the random primer can be the average (it may be a simple average or weight average by taking the amount of nucleotides into consideration).

Specifically, a nucleic acid amplification reaction is carried out with the use of a random primer with a 9 to 30 base length at 4 to 200 microM, and preferably at 4 to 100 microM. Under such conditions, many amplified fragments, and, in particular, many amplified fragments of 100 to 500 base length, can be obtained with high reproducibility via a nucleic acid amplification reaction.

More specifically, random primer concentration is preferably 40 to 60 microM when a random primer is of 9 to 10 base length. When a random primer is of 10 to 14 base length, random primer concentration is preferably at a level that satisfies y>3 E+08 x^{-6.974} and 100 microM or lower, provided that “y” represents the base length of the random primer and “x” represents random primer concentration. When a random primer is of 14 to 18 base length, random primer concentration is preferably 4 to 100 microM. When a random primer is of 18 to 28 base length, it is preferable that random primer concentration be 4 microM or higher and satisfies y<8 E+08 x ^{-5.533}. When a random primer is of 28 to 29 base length, random primer concentration is preferably 6 to 10 microM. By adjusting the random primer concentration at the level described above in accordance with the base length of the random primer, many amplified fragments can be obtained more definitely while achieving high reproducibility.

The inequations described above; i.e., y>3 E+08 x^{-6.974} and y<8 E+08 x^{-5.533}, were formed as a result of thorough investigation of the correlation between the length and the concentration of a random primer as described in WO 2018/003220, so that many DNA fragments of 100 to 500 base length can be amplified with high reproducibility.

The amount of genomic DNA used as a template in a nucleic acid amplification reaction is not particularly limited. When the amount of the reaction solution is 50 microliters, the amount of genomic DNA is preferably 0.1 to 1,000 ng, more preferably 1 to 500 ng, further preferably 5 to 200 ng, and most preferably 10 to 100 ng. By adjusting the amount of genomic DNA used as a template in such range, an amplification reaction from the random primer would not be inhibited, and many amplified fragments can be obtained with high reproducibility.

In accordance with the method described above, a DNA library can be produced from sugarcane with excellent smut resistance, and a DNA library can be produced from sugarcane with smut susceptibility. The nucleotide sequences in these DNA libraries are analyzed with the use of a next-generation sequencer, the read number of fragments constituting the library is compared with each other, and fragments peculiar to the DNA library produced from sugarcane with excellent smut resistance can be selected.

More specifically, the present inventors subjected a wild-type sugarcane line (Iriomote 15) to crossbreeding with a known sugarcane variety (NiF8) to obtain progeny lines (3 lines), subjected the obtained progeny lines to crossbreeding with the wild-type sugarcane line (JW90) to obtain progeny lines (33 lines, 35 line, and 35 lines, respectively), and prepared a DNA library thereof. The resulting DNA library was applied to a next-generation sequencer to obtain the read number data, the genotype data were obtained therefrom, and position information of the marker in the chromosome was determined based on the genotype data using the AntMap software for constructing genetic linkage maps (Iwata H, Ninomiya S, 2006, AntMap: Constructing genetic linkage maps using an ant colony optimization algorithm, Breed Sci., 56: 371-378) in accordance with the Kosambi's genetic distance formula. Further, a genetic map datasheet was prepared based on the obtained marker position information using Mapmaker/EXP ver. 3.0 (A Whitehead Institute for Biomedical Research Technical Report, Third Edition, January, 1993). As a result, the 31,191 markers including the aforementioned 6 types of markers associated with sugarcane smut resistance shown in SEQ ID NOs: 1 to 6 were identified. In addition, the step of obtaining the read number data by applying the DNA library to the next-generation sequencer was repeated two times to obtain a larger amount of the read number data, and the genotype data were obtained from the read number data. Thus, 64,757 markers including 9 types of the markers associated with sugarcane smut resistance as shown in SEQ ID NOs: 135 to 143 were identified in the same manner.
In addition, the present inventors subjected a wild-type sugarcane line (Iriomote 8) to crossbreeding with a known sugarcane variety (NiF8) to obtain progeny lines, subjected a sugarcane variety “NiTn18” to crossbreeding with a sugarcane variety “NiTn24” to obtain progeny lines, obtained 154 progeny lines therefrom, and prepared a DNA library thereof. The resulting DNA library was applied to a next-generation sequencer to obtain the read number data, the genotype data were obtained therefrom, and position information of the marker in the chromosome was determined based on the genotype data using the AntMap software for constructing genetic linkage maps (Iwata H, Ninomiya S, 2006, AntMap: Constructing genetic linkage maps using an ant colony optimization algorithm, Breed Sci., 56: 371-377) in accordance with the Kosambi's genetic distance formula. Further, a genetic map datasheet was prepared based on the obtained marker position information using Mapmaker/EXP ver. 3.0 (A Whitehead Institute for Biomedical Research Technical Report, Third Edition, January, 1993). As a result, 57,444 markers including 7 types of the markers associated with sugarcane smut resistance as shown in SEQ ID NOs: 144 (and 145) to 151 indicated above were identified.

The adjacent region comprising the marker associated with sugarcane smut resistance having the nucleotide sequence as shown in SEQ ID NO: 151 and the adjacent region comprising the marker associated with sugarcane smut resistance having the nucleotide sequence as shown in SEQ ID NO: 1 were identified independently of each other; however, these regions comprise a plurality of markers having the identical nucleotide sequence.

[Use of Markers Associated with Sugarcane Smut Resistance]

With the use of markers associated with sugarcane smut resistance, whether or not a sugarcane progeny line or the like whose phenotype concerning smut resistance remains unknown would exhibit a phenotype for improved smut resistance can be determined. As the marker associated with sugarcane smut resistance, one or more nucleic acid regions included in the 8.4 cM region mentioned above may be used. Alternatively, one or more nucleic acid regions included in the 26.6 cM region mentioned above may be used as the markers associated with sugarcane smut resistance. Further, one or more nucleic acid regions included in the 12.27 cM region may be used as the markers associated with sugarcane smut resistance. Furthermore, one or more nucleic acid regions included in the 8.4 cM region, one or more nucleic acid regions included in the 26.6 cM region, or one or more nucleic acid regions included in the 12.27 cM region may be used as the markers associated with sugarcane smut resistance. In this case, the use of the markers associated with sugarcane smut resistance encompasses an embodiment involving a nucleic acid amplification reaction with the use of a pair of primers that specifically amplifies the markers and an embodiment involving the use of a DNA microarray having probes corresponding to the markers.

A pair of primers that specifically amplifies the markers associated with sugarcane smut resistance can be adequately designed in accordance with the nucleotide sequence of the 8.4 cM region, the nucleotide sequence of the 26.6 cM region, and the nucleotide sequence of the 12.27 cM region. For example, a pair of primers can be designed to amplify a region included in the nucleotide sequence of the 8.4 cM region, the nucleotide sequence of the 26.6 cM region, and the nucleotide sequence of the 12.27 cM region, such as a region of 1 kbp or smaller, 800 bp or smaller, 500 bp or smaller, or 350 bp or smaller. Alternatively, a pair of primers can be designed to amplify a part of or the entire nucleic acid region comprising the nucleotide sequence as shown in any of SEQ ID NO: 1 to 6, 135 to 143, and 144 (145) to 151. A part of the nucleic acid region can be composed of 10 continuous bases, 20 continuous bases, 40 continuous bases, 80 continuous bases, 100 continuous bases, or 140 continuous bases included in the nucleotide sequence as shown in any of SEQ ID NO: 1 to 6, 135 to 143, and 144 (145) to 151.

A probe corresponding to the marker associated with sugarcane smut resistance is an oligonucleotide that can specifically hybridize under stringent conditions to the marker associated with sugarcane smut resistance as defined above. For example, such oligonucleotide can be designed as a partial region of at least 10 continuous bases, 15 continuous bases, 20 continuous bases, 25 continuous bases, 30 continuous bases, 35 continuous bases, 40 continuous bases, 45 continuous bases, or 50 continuous bases of the entire region of the nucleotide sequence of the marker associated with sugarcane smut resistance as defined above or a complementary strand thereof. The probe can be immobilized on a support. Specifically, any type of microarray, such as a microarray having a planar substrate made of glass or silicone as a carrier, a bead array comprising microbeads as carriers, or a three-dimensional microarray comprising a probe immobilized on an inner wall of a hollow fiber, can be used.

With the use of the DNA microarray thus prepared, whether or not a sugarcane line whose phenotype concerning smut resistance remains unknown as typified by a progeny line or the like would exhibit a phenotype for improved smut resistance can be determined. By any method other than the method involving the use of a DNA microarray as described above, whether or not a sugarcane line whose phenotype concerning smut resistance remains unknown would exhibit a phenotype for improved smut resistance may be determined by detecting the marker associated with sugarcane smut resistance in accordance with a conventional technique.

More specifically, genomic DNA is first extracted from a sugarcane sample. In this case, a sugarcane sample is a sugarcane line such as a sugarcane progeny line whose phenotype concerning smut resistance remains unknown and/or a parent sugarcane line used for producing a progeny line. Such sugarcane lines are to be evaluated as to have a trait for improved smut resistance. Also, plants other than sugarcane, such as graminaceous plants including Sorghum or Erianthus, may be employed as plant samples and smut resistance of such plant samples may be evaluated.

Subsequently, a nucleic acid amplification reaction is carried out with the use of the extracted genomic DNA as a template and the pair of primers described above, so as to amplify the marker associated with sugarcane smut resistance. In this case, one of the primers may be labeled with a fluorescent dye and so on, so that the amplified genomic DNA fragment can be labeled. Any conventional substance may be used as a label. Examples of labels that can be used include fluorescent molecules, dye molecules, radioactive molecules, and so on.

Subsequently, a labeled genomic DNA fragment is brought into contact with the DNA microarray under given conditions, so as to allow a probe immobilized on the DNA microarray to hybridize to the labeled genomic DNA fragment. In such a case, hybridization is preferably carried out under highly stringent conditions. Thus, whether or not a sugarcane sample has the marker associated with sugarcane smut resistance can be determined with higher accuracy. In addition, stringent conditions can be adjusted in terms of reaction temperature and salt concentration. That is, higher stringency can be realized by raising temperature or lowering salt concentration. When a probe of 50- to 75-base length is used, for example, higher stringency can be realized by performing hybridization at 40 degrees C. to 44 degrees C. in 0.21 SDS and 6×SSC.

In addition, hybridization between a probe and a labeled genomic DNA fragment can be confirmed by detecting a label. After the above hybridization reaction between the labeled genomic DNA fragment and the probe, specifically, an unreacted genomic DNA fragment or the like is washed, and the label bound to the genomic DNA fragment specifically hybridized to the probe is then observed. When the label is a fluorescent material, for example, the fluorescence wavelength is detected. When the label is a dye molecule, the dye wavelength is detected. More specifically, an apparatus, such as a fluorescent detector or an image analyzer used for conventional DNA microarray analysis, can be used.

It is also possible to detect the marker associated with sugarcane smut resistance in the genomic DNA extracted from a sugarcane sample by a method other than the method involving the use of DNA microarrays described above. For example, the genomic DNA extracted from a sugarcane sample is used as a template and the read number of the marker associated with sugarcane smut resistance is measured using a next-generation sequencer. Thus, the presence or absence of the marker associated with sugarcane smut resistance can be determined with high accuracy.

With the use of the DNA microarray or next-generation sequencer, as described above, whether or not the sugarcane sample has the marker associated with sugarcane smut resistance can be determined. The marker associated with sugarcane smut resistance is linked to a trait of improving smut resistance. If the marker associated with sugarcane smut resistance is present in a sugarcane sample, accordingly, the sugarcane sample can be identified as a variety with improved smut resistance.

According to the method described above, in particular, it is not necessary to cultivate sugarcane samples to such an extent that an actual smut resistance test can be performed. For instance, seeds of a progeny line or a young seedling obtained as a result of germination of such seeds can be used. Therefore, the area of a field used for cultivation of sugarcane samples and other factors such as cost of cultivation can be significantly reduced with the use of the markers associated with sugarcane smut resistance. In addition, use of markers associated with sugarcane smut resistance eliminates the need of actual infection with a causative microorganism of smut (Ustilago scitaminea), and the cost of facilities such as a large-scale special-purpose greenhouse, a special-purpose field, or a facility isolated from an external environment, can be reduced.

When producing a novel sugarcane variety, it is particularly preferable that several tens of thousands of hybrids be first produced via crossbreeding and evaluated with the use of markers associated with sugarcane smut resistance prior to or instead of seedling selection. Thus, the number of elite lines to be cultivated in actual fields can be reduced to a significant extent, and time-consuming efforts and the cost required for production of a novel sugarcane variety can be reduced to a significant extent.

When producing a novel sugarcane variety, alternatively, whether or not a marker associated with sugarcane smut resistance is present in a parent variety subjected to crossbreeding may be first determined, so as to select a parent variety with excellent smut resistance. A parent variety with excellent smut resistance may be preferentially used to produce a progeny line, so that development of a progeny line with excellent smut resistance with high frequency can be expected. Thus, the number of elite lines to be cultivated can be reduced to a significant extent, and time-consuming efforts and the cost required for production of a novel sugarcane variety can be reduced to a significant extent.

### EXAMPLES

The present invention is hereafter described in greater detail with reference to the following examples, although the technical scope of the present invention is not limited thereto.

### Example 1

(1) Materials

Genomic DNAs were extracted from the sugarcane variety (NiF8), the wild-type sugarcane variety (Iriomote 15), 3 progeny lines (KY08-6023), (KY08-6039), and (KY08-6041) resulting from crossbreeding between (NiF8) and (Iriomote 15), and 33, 35, and 35 progeny lines resulting from crossbreeding between (KY08-6023) and the wild-type sugarcane variety (JW90), between (KY08-6039) and the wild-type sugarcane variety (JW90), and between (KY08-6041) and the wild-type sugarcane variety (JW90), respectively. The extracted genomic DNAs were purified with the use of the DNeasy Plant Mini Kit (QIAGEN).

(2) Preparation of DNA library

In this example, a DNA library was prepared in accordance with the method for preparing a DNA library described in WO 2018/003220. Specifically, a dNTP mixture (final concentration 0.2 mM) was added to 15.0 ng of the genomic DNA obtained in (1) above, a 60 microM random primer was added to 0.625 units of Prime STAR DNA Polymerase (Takara Bio Inc.), and the resulting mixtures were each subjected to PCR in the final reaction amount of 25 microliters. PCR was carried out through treatment at 98 degrees C. for 2 minutes, and cycles of 98 degrees C. for 10 seconds, 50 degrees C. for 15 seconds, and 72 degrees C. for seconds, followed by storage at 4 degrees C.

The random primers used in this example are summarized in Table 4.

TABLE 4

| No | Sequence (5′ → 3′) | SEQ ID NO: |
|---|---|---|
| 1 | TAAGAGACAGAGA | 7 |
| 2 | TAAGAGACAGAGC | 8 |
| 3 | TAAGAGACAGGGT | 9 |
| 4 | TAAGAGACAGCTA | 10 |
| 5 | TAAGAGACAGCTC | 11 |
| 6 | TAAGAGACAGGCT | 12 |
| 7 | TAAGAGACAGGTA | 13 |
| 8 | TAAGAGACAGGTC | 14 |
| 9 | TAAGAGACAGTAC | 15 |
| 10 | TAAGAGACAGICA | 16 |
| 11 | TAAGAGACAGTGA | 17 |
| 12 | TAAGAGACAGTTG | 18 |

(3) Preparation of DNA library for next-generation sequencer

A dNTP mixture (final concentration 0.2 mM), 1.25 units of PrimeSTAR HS DNA Polymerase (Takara Bio Inc.), and a set of primers were added to 1.5 microliters of the solution after the reaction of (2) and the resulting mixtures were each subjected to PCR in the final reaction amount of 50 microliters. PCR was carried out through treatment at 95 degrees C. for 2 minutes, and 25 cycles of 98 degrees C. for 15 seconds, 55 degrees C. for 15 seconds, and 72 degrees C. for 20 seconds, followed by storage at 4 degrees C. In this example, the forward primers (SEQ ID NOs: 19 to 133) shown in Table 5 and the reverse primer (5′-AATGATACGGCGACCACCGAGATCTACACCGCGCAGATCGTCGGCAGCGTCAG ATGTGTATAAGAGACAG-3′ (SEQ ID NO: 134)) were used in combination for a total of 115 combinations of 103 lines resulting from crossbreeding between (KY08-6023), (KY08-6039), or (KY08-6041) and (JW90) (33, 35, and 35 lines, respectively) and 6 lines of the parent and grandparent lines (i.e., (NiF8), (Iriomote 15), (KY08-6023), (KY08-6039), (KY08-6041), and (JW90)) (2 repeats each).

TABLE 5

| | | SEQ |
|---|---|---|
| No | Forward (5′ → 3′) | ID NO: |
| 1 | CAAGCAGAAGACGGCATACGAGATTCGTCAGAGTCTCGTGGGCTCGGAGATGTGTATAAGAGACAG | 19 |
| 2 | CAAGCAGAAGACGGCATACGAGATCGCTAGCGGTCTCGTGGGCTCGGAGATGTGTATAAGAGACAG | 20 |
| 3 | CAAGCAGAAGACGGCATACGAGATTCTCAGTAGTCTCGTGGGCTCGGAGATGTGTATAAGAGACAG | 21 |
| 4 | CAAGCAGAAGACGGCATACGAGATCGTAGATAGTCTCGTGGGCTCGGAGATGTGTATAAGAGACAG | 22 |
| 5 | CAAGCAGAAGACGGCATACGAGATACGAGCAGGTCTCGTGGGCTCGGAGATGTGTATAAGAGACAG | 23 |
| 6 | CAAGCAGAAGACGGCATACGAGATATACGTGAGTCTCGTGGGCTCGGAGATGTGTATAAGAGACAG | 24 |
| 7 | CAAGCAGAAGACGGCATACGAGATGTCTAGAGGTCTCGTGGGCTCGGAGATGTGTATAAGAGACAG | 25 |
| 8 | CAAGCAGAAGACGGCATACGAGATAGTCGAGAGTCTCGTGGGCTCGGAGATGTGTATAAGAGACAG | 26 |
| 9 | CAAGCAGAAGACGGCATACGAGATCTCACAGAGTCTCGTGGGCTCGGAGATGTGTATAAGAGACAG | 27 |
| 10 | CAAGCAGAAGACGGCATACGAGATAGACATAGGTCTCGTGGGCTCGGAGATGTGTATAAGAGACAG | 28 |
| 11 | CAAGCAGAAGACGGCATACGAGATAGCGACGCGTCTCGTGGGCTCGGAGATGTGTATAAGAGACAG | 29 |
| 12 | CAAGCAGAAGACGGCATACGAGATTGATAGAGGTCTCGTGGGGTCGGAGATGTGTATAAGAGACAG | 30 |
| 13 | CAAGCAGAAGACGGCATACGAGATGACGACTGGTCTCGTGGGCTCGGAGATGTGTATAAGAGACAG | 31 |
| 14 | CAAGCAGAAGACGGCATACGAGATTGTGCTCAGTCTCGTGGGCTCGGAGATGTGTATAAGAGACAG | 32 |
| 15 | CAAGCAGAAGACGGCATACGAGATATGAGCTGGTCTCGTGGGCTCGGAGATGTGTATAAGAGACAG | 33 |
| 16 | CAAGCAGAAGACGGCATACGAGATTCTCTCACGTCTCGTGGGCTCGGAGATGTGTATAAGAGACAG | 34 |
| 17 | CAAGCAGAAGACGGCATACGAGATGCAGATCAGTCTCGTGGGCTCGGAGATGTGTATAAGAGACAG | 35 |
| 18 | CAAGCAGAAGACGGCATACGAGATTCTGCAGAGTCTCGTGGGCTCGGAGATGTGTATAAGAGACAG | 36 |
| 19 | CAAGCAGAAGACGGCATACGAGATACGTGAGCGTGTCGTGGGGTCGGAGATGTGTATAAGAGACAG | 37 |
| 20 | CAAGCAGAAGACGGCATACGAGATCGCGTGAGGTCTCGTGGGCTCGGAGATGTGTATAAGAGACAG | 38 |
| 21 | CAAGCAGAAGACGGCATACGAGATCATACTACGTCTCGTGGGCTCGGAGATGTGTATAAGAGACAG | 39 |
| 22 | CAAGCAGAAGACGGCATACGAGATTCTACACAGTCTCGTGGGCTCGGAGATGTGTATAAGAGACAG | 40 |
| 23 | CAAGCAGAAGACGGCATACGAGATGATAGATCGTCTCGTGGGCTCGGAGATGTGTATAAGAGACAG | 41 |
| 24 | CAAGCAGAAGACGGCATACGAGATGAGCGTACGTCTCGTGGGCTCGGAGATGTGTATAAGAGACAG | 42 |
| 25 | CAAGCAGAAGACGGCATACGAGATCAGAGACAGTCTCGTGGGCTCGGAGATGTGTATAAGAGACAG | 43 |
| 26 | CAAGCAGAAGACGGCATACGAGATCATAGATGGTCTCGTGGGCTCGGAGATGTGTATAAGAGACAG | 44 |
| 27 | CAAGCAGAAGACGGCATACGAGATAGATGCTAGTCTCGTGGGCTCGGAGATGTGTATAAGAGACAG | 45 |
| 28 | CAAGCAGAAGACGGCATACGAGATCTCATCTCGTCTCGTGGGCTCGGAGATGTGTATAAGAGACAG | 46 |
| 29 | CAAGCAGAAGACGGCATACGAGATTATCTATGGTCTCGTGGGCTCGGAGATGTGTATAAGAGACAG | 47 |
| 30 | CAAGCAGAAGACGGCATACGAGATAGAGTATCGTCTCGTGGGCTCGGAGATGTGTATAAGAGACAG | 48 |
| 31 | CAAGCAGAAGACGGCATACGAGATGTGACTAGGTCTCGTGGGCTCGGAGATGTGTATAAGAGACAG | 49 |
| 32 | CAAGCAGAAGACGGCATACGAGATCTATGCTCGTCTCGTGGGCTCGGAGATGTGTATAAGAGACAG | 50 |
| 33 | CAAGCAGAAGACGGCATACGAGATCTGACTACGTCTCGTGGGCTCGGAGATGTGTATAAGAGACAG | 51 |
| 34 | CAAGCAGAAGACGGCATACGAGATTATCAGTAGTCTCGTGGGCTCGGAGATGTGTATAAGAGACAG | 52 |
| 35 | CAAGCAGAAGACGGCATACGAGATGAGTCTGCGTCTCGTGGGCTCGGAGATGTGTATAAGAGACAG | 53 |
| 36 | CAAGCAGAAGACGGCATACGAGATCAGTCGCAGTCTCGTGGGCTCGGAGATGTGTATAAGAGAGAG | 54 |
| 37 | CAAGCAGAAGACGGCATACGAGATGACATCTAGTCTCGTGGGCTCGGAGATGTGTATAAGAGACAG | 55 |
| 38 | CAAGCAGAAGACGGCATACGAGATCTGTGACGGTCTCGTGGGCTCGGAGATGTGTATAAGAGACAG | 56 |
| 39 | CAAGCAGAAGACGGCATACGAGATAAGAGGCAGTCTCGTGGGCTCGGAGATGTGTATAAGAGACAG | 57 |
| 40 | CAAGCAGAAGACGGCATACGAGATCTAGTACGGTCTCGTGGGCTCGGAGATGTGTATAAGAGACAG | 58 |
| 41 | CAAGCAGAAGACGGCATACGAGATAGGAGTCCGTCTCGTGGGCTCGGAGATGTGTATAAGAGACAG | 59 |
| 42 | CAAGCAGAAGACGGCATACGAGATCATGCCTAGTCTCGTGGGCTCGGAGATGTGTATAAGAGACAG | 60 |
| 43 | CAAGCAGAAGACGGCATACGAGATGTAGAGAGGTCTCGTGGGGTCGGAGATGTGTATAAGAGACAG | 61 |
| 44 | CAAGCAGAAGACGGCATACGAGATCCTCTCTGGTCTCGTGGGCTCGGAGATGTGTATAAGAGACAG | 62 |
| 45 | CAAGCAGAAGACGGCATACGAGATAGCGTAGCGTCTCGTGGGCTCGGAGATGTGTATAAGAGACAG | 63 |
| 46 | CAAGCAGAAGACGGCATACGAGATTCCTCTACGTCTCGTGGGCTCGGAGATGTGTATAAGAGACAG | 64 |
| 47 | CAAGCAGAAGACGGCATACGAGATGACGTACAGTCTCGTGGGCTCGGAGATGTGTATAAGAGACAG | 65 |
| 48 | CAAGCAGAAGACGGCATACGAGATGACTGTAGGTCTCGTGGGCTCGGAGATGTGTATAAGAGACAG | 66 |
| 49 | CAAGCAGAAGACGGCATACGAGATTCAGTACAGTCTCGTGGGCTCGGAGATGTGTATAAGAGACAG | 67 |
| 50 | CAAGCAGAAGACGGCATACGAGATCATGATAGGTCTCGTGGGCTCGGAGATGTGTATAAGAGACAG | 68 |
| 51 | CAAGCAGAAGACGGCATACGAGATGCATCTCGGTCTCGTGGGCTCGGAGATGTGTATAAGAGACAG | 69 |
| 52 | CAAGCAGAAGACGGCATACGAGATTGCACAGAGTCTCGTGGGCTCGGAGATGTGTATAAGAGACAG | 70 |
| 53 | CAAGCAGAAGACGGCATACGAGATGAGCTATAGTCTCGTGGGCTCGGAGATGTGTATAAGAGACAG | 71 |
| 54 | CAAGCAGAAGACGGCATACGAGATAGTCTGCAGTCTCGTGGGCTCGGAGATGTGTATAAGAGACAG | 72 |
| 55 | CAAGCAGAAGACGGCATACGAGATCGCTGTGAGTCTCGTGGGCTCGGAGATGTGTATAAGAGACAG | 73 |
| 56 | CAAGCAGAAGACGGCATACGAGATCTGATGTGGTCTCGTGGGCTCGGAGATGTGTATAAGAGACAG | 74 |
| 57 | CAAGCAGAAGACGGCATACGAGATGCACTCTAGTCTCGTGGGCTCGGAGATGTGTATAAGAGACAG | 75 |
| 58 | CAAGCAGAAGACGGCATACGAGATTCTGCTCAGTCTCGTGGGCTCGGAGATGTGTATAAGAGACAG | 76 |
| 59 | CAAGCAGAAGACGGCATACGAGATTGTATCTAGTCTCGTGGGGTCGGAGATGTGTATAAGAGACAG | 77 |
| 60 | CAAGCAGAAGACGGCATACGAGATACAGTGAGGTCTCGTGGGCTCGGAGATGTGTATAAGAGACAG | 78 |
| 61 | CAAGCAGAAGACGGCATACGAGATATGCGATAGTCTCGTGGGCTCGGAGATGTGTATAAGAGACAG | 79 |
| 62 | CAAGCAGAAGACGGCATACGAGATGAGACATGGTCTCGTGGGCTCGGAGATGTGTATAAGAGACAG | 80 |
| 63 | CAAGCAGAAGACGGCATACGAGATGTCATGTGGTCTCGTGGGCTCGGAGATGTGTATAAGAGACAG | 81 |
| 64 | CAAGCAGAAGACGGCATACGAGATTCATGATAGTCTCGTGGGCTCGGAGATGTGTATAAGAGACAG | 82 |
| 65 | CAAGCAGAAGACGGCATACGAGATGTCATCTCGTCTCGTGGGCTCGGAGATGTGTATAAGAGACAG | 83 |
| 66 | CAAGCAGAAGACGGCATACGAGATTATCTCTAGTCTCGTGGGCTCGGAGATGTGTATAAGAGACAG | 84 |
| 67 | CAAGCAGAAGACGGCATACGAGATCTGATATAGTCTCGTGGGCTCGGAGATGTGTATAAGAGACAG | 85 |
| 68 | CAAGCAGAAGACGGCATACGAGATTAGGCATGGTCTCGTGGGCTCGGAGATGTGTATAAGAGACAG | 86 |
| 69 | CAAGCAGAAGACGGCATACGAGATCGTGAGTGGTCTCGTGGGCTCGGAGATGTGTATAAGAGACAG | 87 |
| 70 | CAAGCAGAAGACGGCATACGAGATGACACATGGTCTCGTGGGCTCGGAGATGTGTATAAGAGACAG | 88 |
| 71 | CAAGCAGAAGACGGCATACGAGATACATGACGGTCTCGTGGGCTCGGAGATGTGTATAAGAGACAG | 89 |
| 72 | CAAGCAGAAGACGGCATACGAGATGCGTCTAGGTCTCGTGGGCTCGGAGATGTGTATAAGAGACAG | 90 |
| 73 | CAAGCAGAAGACGGCATACGAGATTCACGCTGGTCTCGTGGGCTCGGAGATGTGTATAAGAGACAG | 91 |
| 74 | CAAGCAGAAGACGGCATACGAGATTCATGTACGTGTCGTGGGCTCGGAGATGTGTATAAGAGACAG | 92 |
| 75 | CAAGCAGAAGACGGCATACGAGATTAGTGACGGTCTCGTGGGCTCGGAGATGTGTATAAGAGACAG | 93 |
| 76 | CAAGCAGAAGACGGCATACGAGATCACGATAGGTCTCGTGGGCTCGGAGATGTGTATAAGAGACAG | 94 |
| 77 | CAAGCAGAAGACGGCATACGAGATACACACTGGTCTCGTGGGCTCGGAGATGTGTATAAGAGACAG | 95 |
| 78 | CAAGCAGAAGACGGCATACGAGATAGCATCACGTCTCGTGGGGTCGGAGATGTGTATAAGAGACAG | 96 |
| 79 | CAAGCAGAAGACGGCATACGAGATTAGTCGTAGTCTCGTGGGCTGGGAGATGTGTATAAGAGACAG | 97 |
| 80 | CAAGCAGAAGACGGCATACGAGATGCATCGCGGTCTCGTGGGCTCGGAGATGTGTATAAGAGACAG | 98 |
| 81 | CAAGCAGAAGACGGCATACGAGATATCATGTCGTCTCGTGGGCTCGGAGATGTGTATAAGAGACAG | 99 |
| 82 | CAAGCAGAAGACGGCATACGAGATGTACTCTAGTCTCGTGGGCTCGGAGATGTGTATAAGAGACAG | 100 |
| 83 | CAAGCAGAAGACGGCATACGAGATAGTGCATAGTCTCGTGGGCTCGGAGATGTGTATAAGAGACAG | 101 |
| 84 | CAAGCAGAAGACGGCATACGAGATCGCATCAGGTCTCGTGGGCTCGGAGATGTGTATAAGAGACAG | 102 |
| 85 | CAAGCAGAAGACGGCATACGAGATCGCTATAGGTCTCGTGGGCTCGGAGATGTGTATAAGAGACAG | 103 |
| 86 | CAAGCAGAAGACGGCATACGAGATTAGCTCTAGTCTCGTGGGCTCGGAGATGTGTATAAGAGACAG | 104 |
| 87 | CAAGCAGAAGACGGCATACGAGATGTCGATAGGTCTCGTGGGCTCGGAGATGTGTATAAGAGACAG | 105 |
| 88 | CAAGCAGAAGACGGCATACGAGATAGCTCGTAGTCTCGTGGGCTCGGAGATGTGTATAAGAGACAG | 106 |
| 89 | CAAGCAGAAGACGGCATACGAGATACACAGTAGTCTCGTGGGCTCGGAGATGTGTATAAGAGACAG | 107 |
| 90 | CAAGCAGAAGACGGCATACGAGATCAGATGTAGTCTCGTGGGCTCGGAGATGTGTATAAGAGACAG | 108 |
| 91 | CAAGCAGAAGACGGCATACGAGATCTCTACAGGTCTCGTGGGCTCGGAGATGTGTATAAGAGACAG | 109 |
| 92 | CAAGCAGAAGACGGCATACGAGATGTCACTAGGTCTCGTGGGCTCGGAGATGTGTATAAGAGACAG | 110 |
| 93 | CAAGCAGAAGACGGCATACGAGATTGTACTAGGTCTCGTGGGCTCGGAGATGTGTATAAGAGACAG | 111 |
| 94 | CAAGCAGAAGACGGCATACGAGATACGCTATGGTCTCGTGGGCTCGGAGATGTGTATAAGAGACAG | 112 |
| 95 | CAAGCAGAAGACGGCATACGAGATATGTATAGGTCTCGTGGGCTCGGAGATGTGTATAAGAGACAG | 113 |
| 96 | CAAGCAGAAGACGGCATACGAGATTGTGACAGGTCTCGTGGGCTCGGAGATGTGTATAAGAGACAG | 114 |
| 97 | CAAGCAGAAGACGGCATACGAGATGACGTCAGGTCTCGTGGGCTCGGAGATGTGTATAAGAGACAG | 115 |
| 98 | CAAGCAGAAGACGGCATACGAGATAGATCGAGGTCTCGTGGGCTCGGAGATGTGTATAAGAGACAG | 116 |
| 99 | CAAGCAGAAGACGGCATACGAGATATAGTAGAGTCTCGTGGGCTCGGAGATGTGTATAAGAGACAG | 117 |
| 100 | CAAGCAGAAGACGGCATACGAGATTATGACTAGTCTCGTGGGCTCGGAGATGTGTATAAGAGACAG | 118 |
| 101 | CAAGCAGAAGACGGCATACGAGATTAGAGATGGTCTCGTGGGCTCGGAGATGTGTATAAGAGACAG | 119 |
| 102 | CAAGCAGAAGACGGCATACGAGATAGCTGAGAGTCTCGTGGGCTCGGAGATGTGTATAAGAGACAG | 120 |
| 103 | CAAGCAGAAGACGGCATACGAGATACATCTGAGTCTCGTGGGCTCGGAGATGTGTATAAGAGACAG | 121 |
| 104 | CAAGCAGAAGACGGCATACGAGATGCGTGCTAGTCTCGTGGGCTCGGAGATGTGTATAAGAGACAG | 122 |
| 105 | CAAGCAGAAGACGGCATACGAGATACATGTAGGTCTCGTGGGCTCGGAGATGTGTATAAGAGACAG | 123 |
| 106 | CAAGCAGAAGACGGCATACGAGATATAGAGAGGTCTCGTGGGCTCGGAGATGTGTATAAGAGACAG | 124 |
| 107 | CAAGCAGAAGACGGCATACGAGATGTATCTAGGTCTCGTGGGCTCGGAGATGTGTATAAGAGACAG | 125 |
| 108 | CAAGCAGAAGACGGCATACGAGATATACTGTAGTCTCGTGGGCTCGGAGATGTGTATAAGAGACAG | 126 |
| 109 | CAAGCAGAAGACGGCATACGAGATGCACATAGGTCTCGTGGGCTCGGAGATGTGTATAAGAGACAG | 127 |
| 110 | CAAGCAGAAGACGGCATACGAGATATGATGAGGTCTCGTGGGCTCGGAGATGTGTATAAGAGACAG | 128 |
| 111 | CAAGCAGAAGACGGCATACGAGATAGTAGTAGGTCTCGTGGGCTCGGAGATGTGTATAAGAGACAG | 129 |
| 112 | CAAGCAGAAGACGGCATACGAGATTATGTCAGGTCTCGTGGGGTCGGAGATGTGTATAAGAGACAG | 130 |
| 113 | CAAGCAGAAGACGGCATACGAGATGTGTGTGAGTCTCGTGGGCTCGGAGATGTGTATAAGAGACAG | 131 |
| 114 | CAAGCAGAAGACGGCATACGAGATTACGACAGGTCTCGTGGGCTCGGAGATGTGTATAAGAGACAG | 132 |
| 115 | CAAGCAGAAGACGGCATACGAGATATGTGATGGTCTCGTGGGCTCGGAGATGTGTATAAGAGACAG | 133 |

(4) Purification and electrophoresis

Equivalent amounts of the solutions after the reaction in (3) above were mixed in a tube, 50 microliters were separated therefrom and purified using the MinElute PCR Purification Kit (QIAGEN), and the resultant was electrophoresed using the Agilent 2100 bioanalyzer (Agilent Technology) to obtain a fluorescence unit (FU).

(5) Analysis using next-generation sequencer

The DNA library obtained in (3) was analyzed using the Hiseq4000 Sequence System (Illumina) using paired-end 100 bp reads.

(6) Analysis of read data

The read data obtained in (5) above were analyzed using analytical software (GRAS-Di, Toyota Motor Corporation) to obtain the genotype data of 31,191 markers.

On the basis of the genotype data obtained from JW90 in (6) above, genetic map data comprising 86 linkage groups were obtained with the use of the AntMap software for constructing genetic linkage maps (Iwata H, Ninomiya, S., 2006, AntMap: Constructing genetic linkage maps using an ant colony optimization algorithm, Breed Sci., 56: 371-377) by calculation using the Kosambi's genetic distance formula. The 86 linkage groups include genotype data of 4,503 markers of JW90.

(8) Acquisition of smut resistance test data

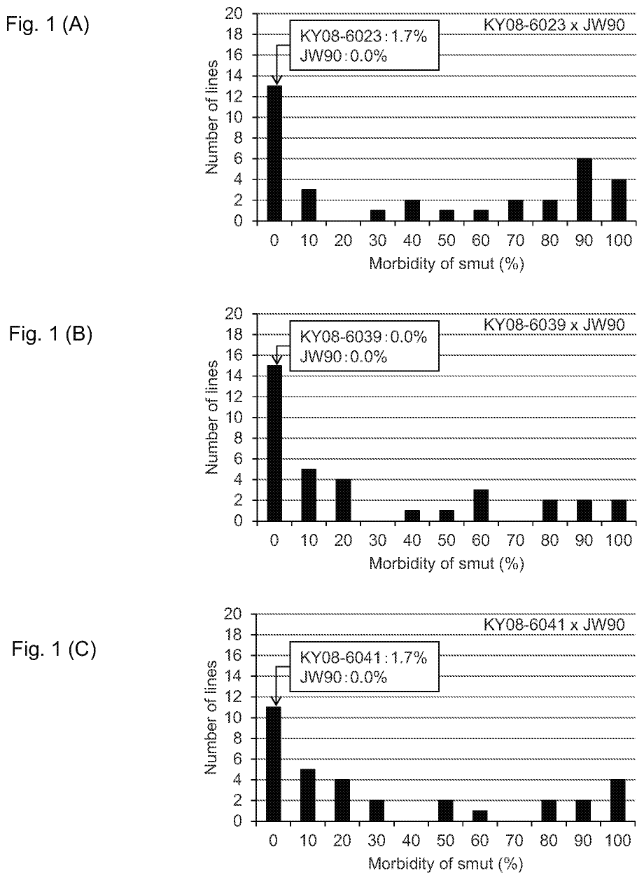

Stalks were collected from the 3 progeny lines resulting from crossbreeding between the sugarcane variety (NiF8) and the wild-type sugarcane variety (Iriomote 15); i.e., (KY08-6023), (KY08-6039), and (KY08-6041), and 33, 35, and 35 progeny lines resulting from crossbreeding between (KY08-6023), (KY08-6039), and (KY08-6041) and the wild-type sugarcane variety (JW90), and the collected stalks were subjected to germination stimulation at room temperature and high humidity for 2 to 3 days, followed by wound inoculation with smut spores. For wound inoculation, wounds were made on both sides of buds (6 wounds in total; approximately 4.0 mm in depth), and a spore suspension (10 7 to 10 8 spores/ml) was then applied to the wounds using a brush. Seedlings subjected to wound inoculation were cultivated for 2 to 3 days at room temperature and high humidity and planted in nursery boxes (40 buds/box, 2 boxes/line). The planted seedlings were cultivated at high humidity in a greenhouse. The degree of the development of smut was investigated by counting, as the number of affected seedlings, the number of seedlings showing a symptom of smut, which is the outgrowth of a smut whip from the apex of a stalk. After the investigation of the number of the affected seedlings, the plant bodies of affected seedlings were harvested at the ground level and removed. The morbidity of smut was calculated as a percentage of the number of germinating stocks (excluding stocks killed by non-smut causes) accounted for by the number of affected stocks. FIG. 1 (A) shows the results of calculation of the morbidity of smut of the progeny line resulting from crossbreeding between (KY08-6023) and (JW90), FIG. 1 (B) shows the results of calculation of the morbidity of smut of the progeny line resulting from crossbreeding between (KY08-6039) and (JW90), and FIG. 1 (C) shows the results of calculation of the morbidity of smut of the progeny line resulting from crossbreeding between (KY08-6041) and (JW90).

(9) Quantitative trait loci (QTL) analysis

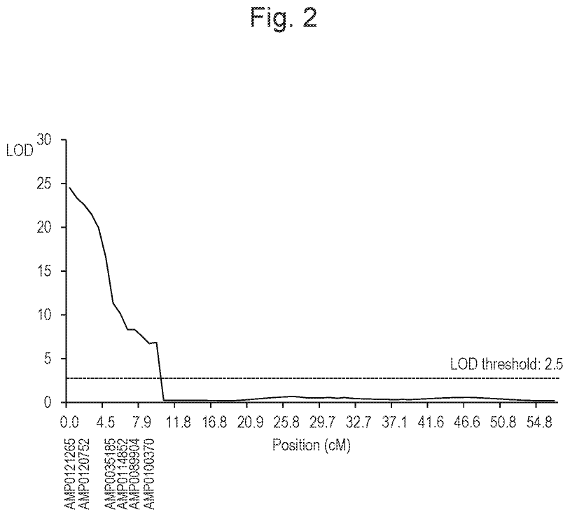

Based on the genetic map data obtained from JW90 in (7) above and the smut resistance test data obtained in (8) above, QTL analysis was carried out by the composite interval mapping (CIM) method using the QTL Cartographer gene analysis software (statgen.ncsu.edu/qticart/cartographerhtml). The LOD threshold was determined to be 2.5. As a result, the presence of QTL linked to sugarcane smut resistance was confirmed in a region of approximately 8.4 cM including the markers AMP0121265 to AMP0100370 in the 42nd linkage group of the wild-type sugarcane variety (JW90) (Table 6 and FIG. 2). When the value indicating the effect is negative, QTL is linked to a trait of improving smut resistance.

TABLE 6

| | Linkage | Position | Range | | LOD | Effects |
|---|---|---|---|---|---|---|
| Varity | group | (cM) | (cM) | Adjacent marker | value | (%) |
| JW90 | 42 | 0.0 | 8.4 | AMP0121265- | 24.5 | −47.2 |
| | | | | AMP0100370 | | |

As shown in Table 6 and FIG. 2, the range including the markers AMP0121265 to AMP0100370 in the 42nd linkage group observed in the present example exhibits a significantly higher LOD value and significantly improved effects, compared with those described in WO 2012/147635.

(10) Selection of smut resistance selection marker

The markers included in the QTL region linked to sugarcane smut resistance confirmed in (9) above (i.e., AMP0121265, AMP0120752, AMP0035185, AMP0114852, AMP0089904, and AMP0100370) were selected as selection markers (Table 7).

TABLE 7

| | Linkage | | | SEQ ID |
|---|---|---|---|---|
| Varity | group | Marker | Nucleotide sequence information | NO: |
| JW90 | 42 | AMP0121265 | TAGCCCACTAAAAGAAAGCCTTGCATAACCCTTGATGTCA | SEQ ID |
| | | | CTTTATTTTGGTTTAAGACAGATAAGTCTAGCTGAGTACCT | NO: 1 |
| | | | TCTCGTACTTAGGGCGTTGTTCCCATTGTTGTTGTAGATG | |
| | | | ATTAGATGTACTACGGCTATTGCGTCA | |
| JW90 | 42 | AMP0120752 | TAGAGCGGAGGGCGTTGGAGCGTCTAGGAATAGATCGCG | SEQ ID |
| | | | TTCTCTCCAGTGGCGAGCCGATCTGAGTGGGAGGACGT | NO: 2 |
| | | | GGATGGGGCTCACGCGGCGAGGAGGATGTGGATGCGGC | |
| | | | GCCGTCCTGGTTCCTGCTCAACCACAGCGACGCTGGCG | |
| | | | GCTACTGCACGGTGGA | |
| JW90 | 42 | AMP0035185 | CTCGAGCGGTGTCGGAGAGGAACAGGGGGAGCTGCCG | SEQ ID |
| | | | GATGATGACCCGGTCGTCGGTGGCCCCACCCAGCTGAC | NO: 3 |
| | | | ACGCCAAGCGGAAATCGGCGAGCCAAAGTTCGGGTTTG | |
| | | | GTCTCGCTGTTGTACTTGGTGAGAGTCGTGGGGGGCGA | |
| | | | AACCGCGCCGGGAGTATCATTTCACGAATAGCCTTACTGA | |
| | | | AGACCCGAGGGCCCGGAGGCTCGGGTGAAGGGCTACGA | |
| | | | TCCTCCGA | |
| JW90 | 42 | AMP0114852 | TACCCCACCAAAAAGCCTATCATGATCCTTGGTGTCATTTT | SEQ ID |
| | | | ATTTTGGTTCTGTCGGGTAAGTCTAGCTGAGTACCTTCTC | NO: 4 |
| | | | GTACTCAGGGCTTTGTTCCCACTTGTTGCAGATGGACAG | |
| | | | ATGTAGTATGGTTATTGTATCA | |
| JW90 | 42 | AMP0089904 | GCCACCGGCCTCGTGGTCGTGAGACCGAGGAGAACGGT | SEQ ID |
| | | | ACCTGGGTATGGGCTGGTAGTCCTCATCCGTGCTGTCAG | NO: 5 |
| | | | AAGTCACATCCTCAGGAAGATCAGCAGGCTGAGCGTCAA | |
| | | | GATCGGCAAGCTCTGCCTCTGCAAGCTCATCAACTGTGG | |
| | | | CGTCTTGCTCAGCTGCTGTCTCAGGAACCTCAGGTCGCT | |
| | | | CAC | |
| JW90 | 42 | AMP0100370 | GCGGTGAATAACGCGCAAAGGGTGAGAAGGCTCACACC | SEQ ID |
| | | | GGATCTACGGGTTACACAGAGGTACACATCCATCGATTCT | NO: 6 |
| | | | AACACGGGCGTCCGGCTCGGCGCCGGGGCGCGGCGAG | |
| | | | GGGTGGAGTGGATAGAACTCGCAGAAGAAGACGTGTTAC | |
| | | | TGTCTGTCCGCAC | |

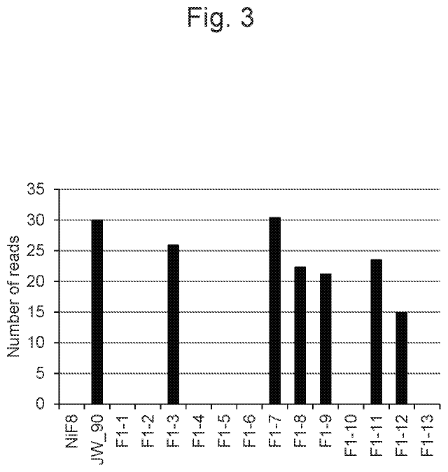

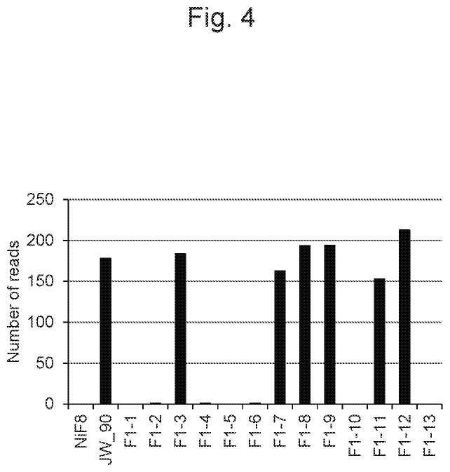

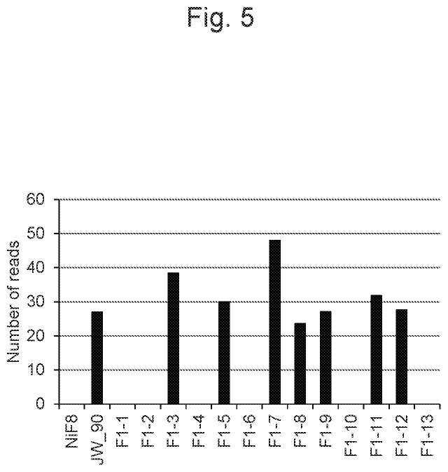

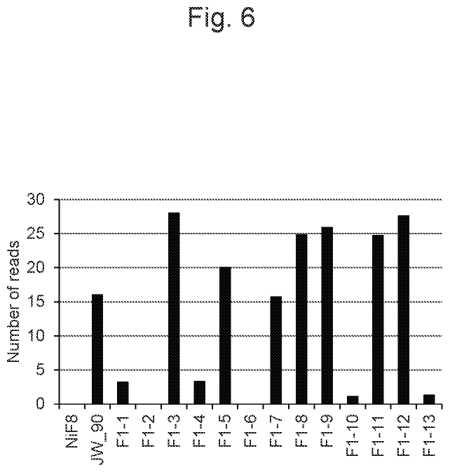

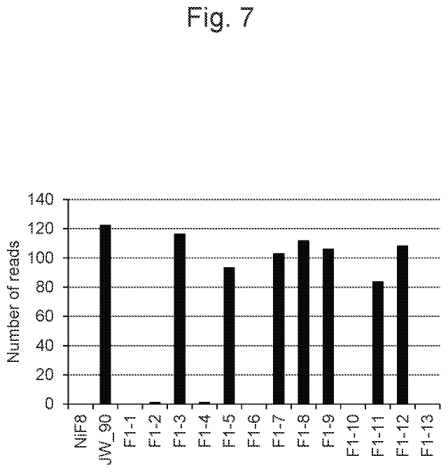

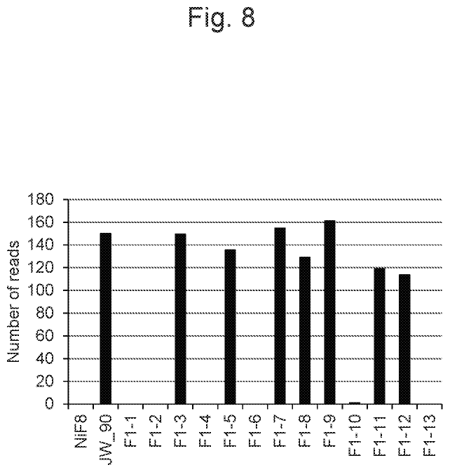

Whether or not genotypes of the markers; i.e., the line samples, have the selection markers was determined by designating the threshold of each number of reads as 10, evaluating as “present” when the number of reads is 10 or more, and evaluating as “absent” when the number of reads is less than 10 (FIGS. 3 to 8, Table 8).

TABLE 8

| Marker | NiF8 | JW_90 | F1-1 | F1-2 | F1-3 | F1-4 | F1-5 | F1-6 | F1-7 | F1-8 | F1-9 | F1-10 | F1-11 | F1-12 | F1-13 |
|---|---|---|---|---|---|---|---|---|---|---|---|---|---|---|---|
| AMP0121265 | 0 | 30 | 0 | 0 | 25.9 | 0 | 0 | 0 | 30.4 | 22.3 | 21.2 | 0 | 23.5 | 14.9 | 0 |
| AMP0120752 | 0 | 178.2 | 0 | 1 | 183.6 | 1.1 | 0 | 1.1 | 162.6 | 193.5 | 194.1 | 0 | 153 | 212.6 | 0 |
| AMP0035185 | 0 | 27 | 0 | 0 | 38.4 | 0 | 30 | 0 | 48 | 23.6 | 27.1 | 0 | 31.8 | 27.6 | 0 |
| AMP0114852 | 0 | 16 | 3.2 | 0 | 28 | 3.3 | 20 | 0 | 15.7 | 24.8 | 25.9 | 1.1 | 24.7 | 27.6 | 1.3 |
| AMP0089904 | 0 | 122.1 | 0 | 1 | 116.2 | 1.1 | 93.3 | 0 | 102.9 | 111.6 | 105.9 | 0 | 83.6 | 108 | 0 |
| AMP0100370 | 0 | 150.2 | 0 | 0 | 149.4 | 0 | 135.5 | 0 | 154.8 | 129 | 161.1 | 1.1 | 118.9 | 113.8 | 0 |
| Incidence of | 40.8 | 0 | 50.9 | 50 | 0 | 70.1 | 87.7 | 90.7 | 0 | 0 | 0 | 80.3 | 0 | 0 | 97.3 |
| smut (%) | | | | | | | | | | | | | | | |

As shown in Table 8 and FIGS. 3 to 8, a line sample exhibiting a lower morbidity of smut has a significantly larger number of markers than a line sample exhibiting a higher morbidity of smut. Such results demonstrate that continuous nucleic acid regions selected from the region of approximately 8.4 cM including AMP0121265, AMP0120752, AMP0035185, AMP0114852, AMP0089904, and AMP0100370 can be used as the markers associated with sugarcane smut resistance.

### Example 2

In Example 1, the marker associated with sugarcane smut resistance was identified based on the genotype data of 4,503 markers of JW90 among the genotype data of the 31,191 markers. In this example, the genotype data of Iriomote 15 were collected, and the marker associated with sugarcane smut resistance was identified in the same manner.

In this example, the DNA library prepared in Example 1 was subjected to the analysis using a next-generation sequencer as described in (5) two times in order to increase the amount of genotype data of Iriomote 15. The read data thus obtained were analyzed using analytical software (GRAS-Di, Toyota Motor Corporation) to obtain the genotype data of 64,757 markers.

On the basis of the genotype data obtained from progeny lines; i.e., KY08-6023, KY08-6039, and KY08-6041, of Iriomote 15, genetic map data comprising 58 linkage groups were obtained with the use of the AntMap software for constructing genetic linkage maps (Iwata H, Ninomiya, S., 2006, AntMap: Constructing genetic linkage maps using an ant colony optimization algorithm, Breed Sci., 56: 371-377) by calculation using the Kosambi's genetic distance formula in the same manner as in Example 1. The 58 linkage groups include genotype data of 4,503 markers of the progeny lines; i.e., KY08-6023, KY08-6039, and KY08-6041, of Iriomote 15.

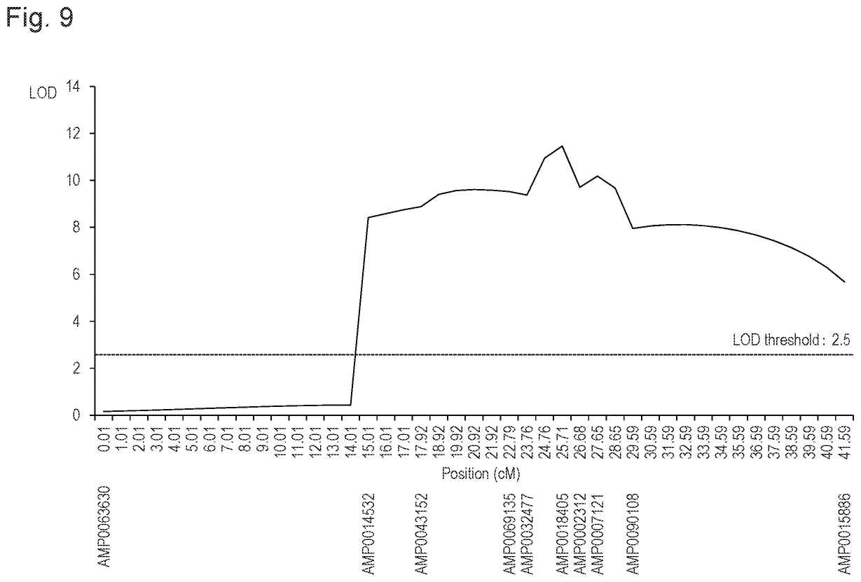

Based on the genetic map data thus obtained and the smut resistance test data obtained in Example 1, QTL analysis was carried out by the composite interval mapping (CIM) method using the QTL Cartographer gene analysis software (statgen.ncsu.edu/qticart/cartographer.html). The LOD threshold was determined to be 2.5. As a result, the presence of QTL linked to sugarcane smut resistance was confirmed in a region between the markers AMP0014532 and AMP0015886 in the 15th linkage group of the wild-type sugarcane variety (Iriomote 15) (Table 9 and FIG. 9). When the value indicating the effect is negative, QTL is linked to a trait of improving the smut resistance.

TABLE 9

| | Linkage | Position | Range | | LOD | Effects |
|---|---|---|---|---|---|---|
| Varity | group | (cM) | (cM) | Adjacent marker | value | (%) |
| Iriomote 15 | 15 | 25.7 | 26.6 | AMP0014532- | 11.5 | −37.8 |
| | | | | AMP0015886 | | |

As shown in Table 9 and FIG. 9, the range including the markers AMP0014532 to AMP0015886 in the 15th linkage group observed in Example 2 was found to exhibit a significantly higher LOD value and significantly improved effects, compared with those described in WO 2012/147635. In this example, the markers included in the QTL region linked to sugarcane smut resistance thus confirmed (i.e., AMP0014532, AMP0043152, AMP0069135, AMP0032477, AMP0018405, AMP0002312, AMP0007121, AMP0090108, and AMP0015886) were selected as selection markers (Table 10).

TABLE 10

| | Linkage | | | SEQ ID |
|---|---|---|---|---|
| Varity | group | Marker | Nucleotide sequence information | NO: |
| Iriomote | 15 | AMP0014532 | CAGAACGTGGTCGCCGTCGCCATGCTCCTTCGTAACAT | SEQ ID |
| 15 | | | GCCTGAGCCATCGAATCCTGACACTCGTCAAGCCCGAG | NO: 135 |
| | | | ATGAAATCCGAGGACTCATCGAGACCGCTGCTATGCAG | |
| | | | CAAACCGAGAGTTCTGCCTTGAGGTGACGCGGGCCTAC | |
| | | | CTCGGAGCAG | |
| Iriomote | 15 | AMP0043152 | CTCTGGCGAACGCGGTGAGGAGCACACCCATGGTCTGC | SEQ ID |
| 15 | | | CCCGGCGAGATGACCAGCGTGGTGGCCGCTGTAGACC | NO: 136 |
| | | | CCCACACGCCGCCCTGCACTCTGCCTCCTTCCGAGGAG | |
| | | | TTCGACCTCTTCGGC | |
| Iriomote | 15 | AMP0069135 | GAGCACTTGATGTCACCTAATATACTGGTTCTGAGCCAT | SEQ ID |
| 15 | | | TACCCGTTTGGGGTCCAATTAATTTATGAAATGACTTGTT | NO: 137 |
| | | | GTCAATTACCCTGAGTTTTTGTCCACTCTGAAGCTGAACT | |
| | | | GAACATTGTGTGCTCCGGTAGCTGAAAATCCTACCCTTA | |
| | | | GCACTCAGACTGAGTCCATTGTTCTTATCATAGCCTTATG | |
| | | | GTTGTTCATTTTGTATGC | |
| Iriomote | 15 | AMP0032477 | CTCGAGCGGTGTCCGACAGGAACAGGGGGAGTTGTCGT | SEQ ID |
| 15 | | | ATGATGACCCGGTCGTCAGTGGCCCCACCCAACTGACA | NO: 138 |
| | | | AGCCAAGTGGAAGTCAGCGAGCCAAAGTTTGGGTTTGG | |
| | | | TTTCGCCGTTATACTTGGTAAGAGTCGTGGGGGGGCGG | |
| | | | AACCGAGCCGGGAGTAACATTTCATGAATGGCCTTGCTA | |
| | | | AAGACCCGAGGTCCCGGTGGCTCGGGTGAAGGGCTTTG | |
| | | | ATCCTCCGA | |
| Iriomote | 15 | AMP0018405 | CAGTAAGCTCCATCGAGGGACAAGCATCTAGTGAGTCAA | SEQ ID |
| 15 | | | TACCAAAGTGGCGTGGGGCATAACCATACAGAGCCTGA | NO: 139 |
| | | | AATGGGGAACGATCCAGAGTGGAATGCCAACTTGAATTG | |
| | | | TACCAAAACTCGGCAAGATGGATCCAATTATGCCACTTG | |
| | | | TGGGGTACCGCATTGACAAAACAGCGTAAGAAGGTCTC | |
| | | | CATGCACTGGTTGACGCGC | |
| Iriomote | 15 | AMP0002312 | ACAGTGAACAGTGGTACTATATACAGTACCCGCTACAGC | SEQ ID |
| 15 | | | AACACACGTCGTGGTCTCCTTAGGCAGCAGAAATGGTAT | NO: 140 |
| | | | AAAAAAACGTACATAAAGGTACATGGCAGATAGGAGTAT | |
| | | | ATTCTATAAGGAGCACGTAACGCGTGACGGGTCACGCG | |
| | | | TTCCAGCGTCGTCTACCTCGTGACAGGCCTCCTCCCTTG | |
| | | | CCCCCTTCTCCCTCCCCGTGCCCCTCCTCCTCTGCCCC | |
| | | | CTCCCTC | |
| Iriomote | 15 | AMP0007121 | CAAAACAGGGACAATATATATCCTTTGTCTGCTTAATCAA | SEQ ID |
| 15 | | | AGCATGCCTTTTAGCTGCTTGAATAAAACCATTCACATCT | NO: 141 |
| | | | TGTATGTAGAAAGCTTCATATCTTCGTTTCGTGTACATCC | |
| | | | ATGA | |
| Iriomote | 15 | AMP0090108 | GCCACCGGCCTCGTGGTCGTGAGACCGAGGAGACCGG | SEQ ID |
| 15 | | | TACCTGGGGATGGGCTGGTAGTCCTCATCAGTGCTGTC | NO: 142 |
| | | | TGAAGTGACATCCTCAGGTATATCAGCAGTCTGAGTCTC | |
| | | | AAGGTCCGCGAGCTCTGCCTCGGCAAGCTCGTCAACTC | |
| | | | TGGCGTCCTGCTCAGCTGCTGTCTCAGGAACATCTGGG | |
| | | | CGCTCAC | |
| Iriomote | 15 | AMP0015886 | CAGAGGCCCTTGAAAGCACAGCTGGTTTTCATCGTCACC | SEQ ID |
| 15 | | | CAGTGAAGAATGACATTTCGAGGAAGGCTTTAGGCTACT | NO: 143 |
| | | | TGAATCCCACGAACATTTCTACAGTAATATTGGTAATATG | |
| | | | CGCAGAGGGTCCTTGGGTACTC | |

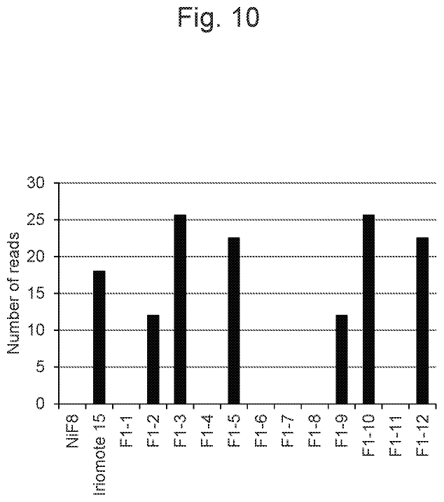

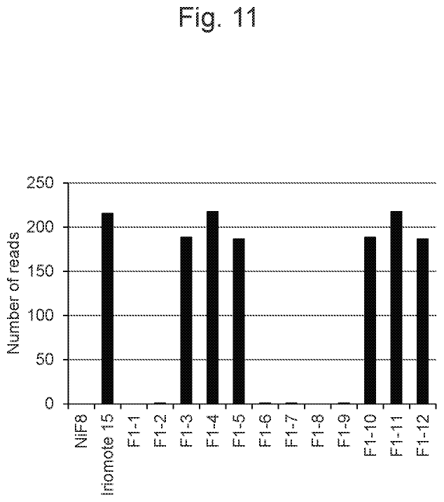

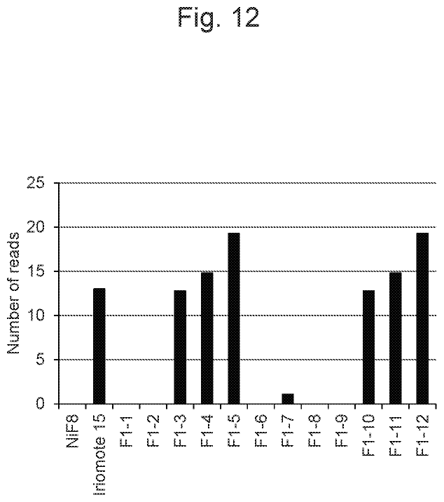

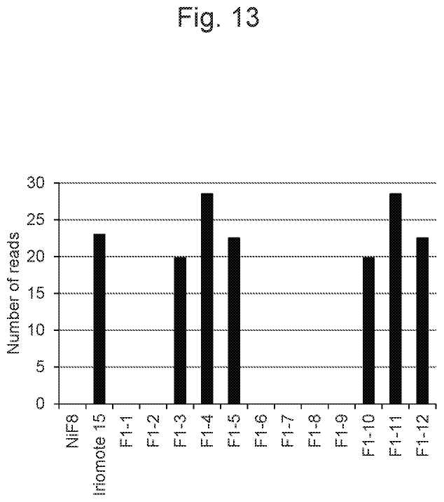

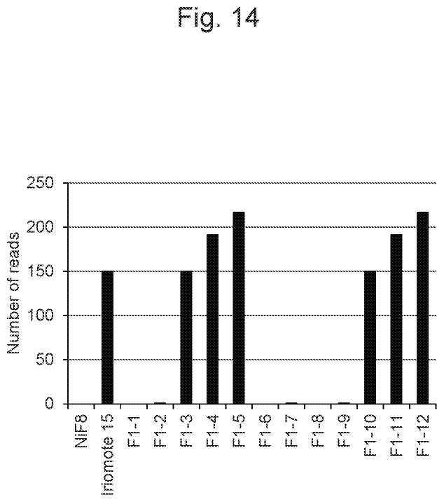

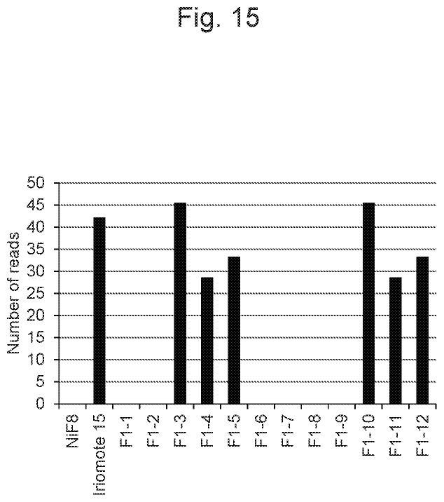

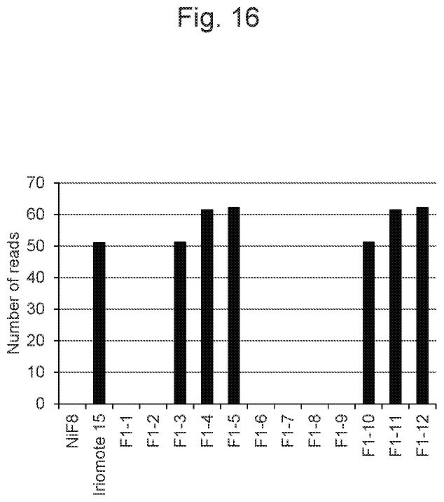

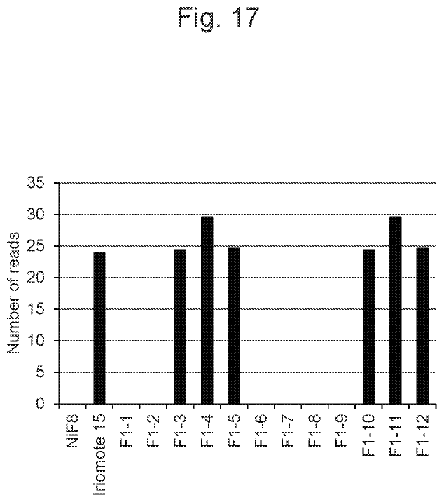

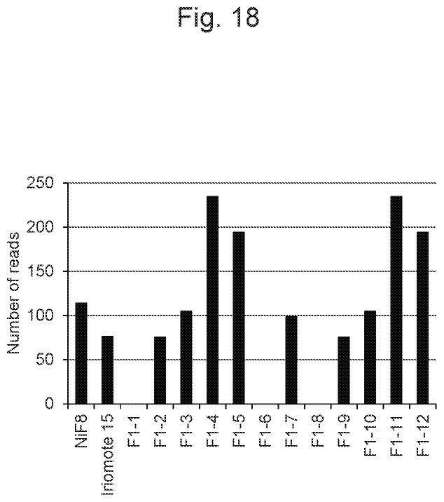

Whether or not genotypes of the markers; i.e., the line samples, have the selection markers was determined by designating the threshold of each number of reads as 10, evaluating as “present” when the number of reads is 10 or more, and evaluating as “absent” when the number of reads is less than 10 (FIGS. 10 to 18, Table 11).

TABLE 11

| Marker | NiF8 | Iriomote 15 | F1-1 | F1-2 | F1-3 | F1-4 | F1-5 | F1-6 | F1-7 | F1-8 | F1-9 | F1-10 | F1-11 | F1-12 |
|---|---|---|---|---|---|---|---|---|---|---|---|---|---|---|
| AMP0014532 | 0 | 18 | 0 | 12 | 25.6 | 0 | 22.5 | 0 | 0 | 0 | 12 | 25.6 | 0 | 22.5 |
| AMP0043152 | 0 | 215.3 | 0 | 1.1 | 188.4 | 217.6 | 186.5 | 1.1 | 1.1 | 0 | 1.1 | 188.4 | 217.6 | 186.5 |
| AMP0069135 | 0 | 13 | 0 | 0 | 12.8 | 14.8 | 19.3 | 0 | 1.1 | 0 | 0 | 12.8 | 14.8 | 19.3 |
| AMP0032477 | 0 | 23 | 0 | 0 | 19.8 | 28.5 | 22.5 | 0 | 0 | 0 | 0 | 19.8 | 28.5 | 22.5 |
| AMP0018405 | 0 | 150.2 | 0 | 1.1 | 150.1 | 191.4 | 216.5 | 0 | 1.1 | 0 | 1.1 | 150.1 | 191.4 | 216.5 |
| AMP0002312 | 0 | 42.1 | 0 | 0 | 45.4 | 28.5 | 33.2 | 0 | 0 | 0 | 0 | 45.4 | 28.5 | 33.2 |
| AMP0007121 | 0 | 51.1 | 0 | 0 | 51.2 | 61.5 | 62.2 | 0 | 0 | 0 | 0 | 51.2 | 61.5 | 62.2 |
| AMP0090108 | 0 | 24 | 0 | 0 | 24.4 | 29.6 | 24.6 | 0 | 0 | 0 | 0 | 24.4 | 29.6 | 24.6 |
| AMP0015886 | 113.9 | 76.1 | 0 | 75.5 | 104.7 | 234.6 | 194 | 0 | 98.6 | 0 | 75.5 | 104.7 | 234.6 | 194 |
| Incidence of | 40.8 | 0 | 97.3 | 89.7 | 0 | 3.6 | 1.5 | 98.6 | 80.3 | 97.3 | 89.7 | 0 | 3.6 | 1.5 |
| smut (%) | | | | | | | | | | | | | | |

As shown in Table 11 and FIGS. 10 to 18, a line sample exhibiting a lower morbidity of smut was found to have a significantly larger number of markers than a line sample exhibiting a higher morbidity of smut. Such results demonstrate that continuous nucleic acid regions selected from the region of approximately 26.6 cM including AMP0014532, AMP0043152, AMP0069135, AMP0032477, AMP0018405, AMP0002312, AMP0007121, AMP0090108, and AMP0015886 can be used as the markers associated with sugarcane smut resistance.

### Example 3

In this example, the marker associated with sugarcane smut resistance derived from the wild-type sugarcane line “Iriomote 8” was identified in the same manner as in Example 1, except for the use of the progeny line “KY09-6092” resulting from crossbreeding between the sugarcane variety “NiF8” and the wild-type sugarcane line “Iriomote 8,” the progeny line “KY08-129” resulting from crossbreeding between the sugarcane variety “NiTn18” and the sugarcane variety “NiN24,” and the 154 progeny lines resulting from crossbreeding between “KY09-6092” and “KY08-129.”

In this example, the read data obtained in the same manner as in Example 1 except for the use of the forward primers shown in Table 12 instead of the forward primers shown in Table 5 for preparation of the DNA library for a next-generation sequencer were analyzed using analytical software (GRAS-Di, Toyota Motor Corporation) to obtain the genotype data of 57,444 markers.

TABLE 12

| | | SEQ |
|---|---|---|
| No | Forward (5′ → 3′) | ID NO: |
| 1 | CAAGCAGAAGACGGCATACGAGATTCGTCAGAGTCTCGTGGGCTCGGAGATGTGTATAAGAGACAG | 152 |
| 2 | CAAGCAGAAGACGGCATACGAGATCGCTAGCGGTCTCGTGGGCTCGGAGATGTGTATAAGAGACAG | 153 |
| 3 | CAAGCAGAAGACGGCATACGAGATTCTCAGTAGTCTCGTGGGCTCGGAGATGTGTATAAGAGACAG | 154 |
| 4 | CAAGCAGAAGACGGCATACGAGATCGTAGATAGTCTCGTGGGCTCGGAGATGTGTATAAGAGACAG | 155 |
| 5 | CAAGCAGAAGACGGCATACGAGATATACGTGAGTCTCGTGGGCTCGGAGATGTGTATAAGAGACAG | 156 |
| 6 | CAAGCAGAAGACGGCATACGAGATGTCTAGAGGTCTCGTGGGCTCGGAGATGTGTATAAGAGACAG | 157 |
| 7 | CAAGCAGAAGACGGCATACGAGATAGTCGACAGTCTCGTGGGCTCGGAGATGTGTATAAGAGACAG | 158 |
| 8 | CAAGCAGAAGACGGCATACGAGATAGCGACGCGTCTCGTGGGCTCGGAGATGTGTATAAGAGACAG | 159 |
| 9 | CAAGCAGAAGACGGCATACGAGATTGATAGAGGTCTCGTGGGCTCGGAGATGTGTATAAGAGACAG | 160 |
| 10 | CAAGCAGAAGACGGCATACGAGATGACGACTGGTCTCGTGGGCTCGGAGATGTGTATAAGAGACAG | 161 |
| 11 | CAAGCAGAAGACGGCATACGAGATTGTGCTCAGTCTCGTGGGCTCGGAGATGTGTATAAGAGACAG | 162 |
| 12 | CAAGCAGAAGACGGCATACGAGATATGAGCTGGTCTCGTGGGCTCGGAGATGTGTATAAGAGACAG | 163 |
| 13 | CAAGCAGAAGACGGCATACGAGATTCTCTCACGTCTCGTGGGCTCGGAGATGTGTATAAGAGACAG | 164 |
| 14 | CAAGCAGAAGACGGCATACGAGATGCAGATCAGTCTCGTGGGCTCGGAGATGTGTATAAGAGACAG | 165 |
| 15 | CAAGCAGAAGACGGCATACGAGATTCTGCAGAGTCTCGTGGGCTCGGAGATGTGTATAAGAGACAG | 166 |
| 16 | CAAGCAGAAGACGGCATACGAGATACGTGAGCGTCTCGTGGGCTCGGAGATGTGTATAAGAGACAG | 167 |
| 17 | CAAGCAGAAGACGGCATACGAGATCGCGTGAGGTCTCGTGGGCTCGGAGATGTGTATAAGAGACAG | 168 |
| 18 | CAAGCAGAAGACGGCATACGAGATCATACTACGTCTCGTGGGCTCGGAGATGTGTATAAGAGACAG | 169 |
| 19 | CAAGCAGAAGACGGCATACGAGATTGTACACAGTCTCGTGGGCTCGGAGATGTGTATAAGAGACAG | 170 |
| 20 | CAAGCAGAAGACGGCATACGAGATGATAGATCGTCTCGTGGGCTCGGAGATGTGTATAAGAGACAG | 171 |
| 21 | CAAGCAGAAGACGGCATACGAGATGAGCGTACGTCTCGTGGGCTCGGAGATGTGTATAAGAGACAG | 172 |
| 22 | CAAGCAGAAGACGGCATACGAGATCAGAGACAGTCTCGTGGGCTCGGAGATGTGTATAAGAGACAG | 173 |
| 23 | CAAGCAGAAGACGGCATACGAGATCATAGATGGTCTCGTGGGCTCGGAGATGTGTATAAGAGACAG | 174 |
| 24 | CAAGCAGAAGACGGCATACGAGATAGATGCTAGTCTCGTGGGCTCGGAGATGTGTATAAGAGACAG | 175 |
| 25 | CAAGCAGAAGACGGCATACGAGATCTCATCTCGTCTCGTGGGCTCGGAGATGTGTATAAGAGACAG | 176 |
| 26 | CAAGCAGAAGACGGCATACGAGATTATCTATGGTCTCGTGGGCTCGGAGATGTGTATAAGAGACAG | 177 |
| 27 | CAAGCAGAAGACGGCATACGAGATAGAGTATCGTCTCGTGGGCTCGGAGATGTGTATAAGAGACAG | 178 |
| 28 | CAAGCAGAAGACGGCATACGAGATGTGACTAGGTCTCGTGGGCTCGGAGATGTGTATAAGAGACAG | 179 |
| 29 | CAAGCAGAAGACGGCATACGAGATCTATGCTCGTCTCGTGGGCTCGGAGATGTGTATAAGAGACAG | 180 |
| 30 | CAAGCAGAAGACGGCATACGAGATCTGACTACGTCTCGTGGGCTCGGAGATGTGTATAAGAGACAG | 181 |
| 31 | CAAGCAGAAGACGGCATACGAGATTATCAGTAGTCTCGTGGGCTCGGAGATGTGTATAAGAGACAG | 182 |
| 32 | CAAGCAGAAGACGGCATACGAGATGAGTCTGCGTCTCGTGGGCTCGGAGATGTGTATAAGAGACAG | 183 |
| 33 | CAAGCAGAAGACGGCATACGAGATCAGTCGCAGTCTCGTGGGCTCGGAGATGTGTATAAGAGACAG | 184 |
| 34 | CAAGCAGAAGACGGCATACGAGATGACATCTAGTCTCGTGGGCTCGGAGATGTGTATAAGAGACAG | 185 |
| 35 | CAAGCAGAAGACGGCATACGAGATCTGTGACGGTCTCGTGGGCTCGGAGATGTGTATAAGAGACAG | 186 |
| 36 | CAAGCAGAAGACGGCATACGAGATAAGAGGCAGTCTCGTGGGCTCGGAGATGTGTATAAGAGACAG | 187 |
| 37 | CAAGCAGAAGACGGCATACGAGATCTAGTACGGTCTCGTGGGCTCGGAGATGTGTATAAGAGACAG | 188 |
| 38 | CAAGCAGAAGACGGCATACGAGATAGGAGTCCGTCTCGTGGGCTCGGAGATGTGTATAAGAGACAG | 189 |
| 39 | CAAGCAGAAGACGGCATACGAGATCATGCCTAGTCTCGTGGGCTCGGAGATGTGTATAAGAGACAG | 190 |
| 40 | CAAGCAGAAGACGGCATACGAGATGTAGAGAGGTCTCGTGGGCTCGGAGATGTGTATAAGAGACAG | 191 |
| 41 | CAAGCAGAAGACGGCATACGAGATCCTCTCTGGTCTCGTGGGCTCGGAGATGTGTATAAGAGACAG | 192 |
| 42 | CAAGCAGAAGACGGCATACGAGATAGCGTAGCGTCTCGTGGGCTCGGAGATGTGTATAAGAGACAG | 193 |
| 43 | CAAGCAGAAGACGGCATACGAGATTCCTCTACGTCTCGTGGGCTCGGAGATGTGTATAAGAGACAG | 194 |
| 44 | CAAGCAGAAGACGGCATACGAGATGACGTACAGTCTCGTGGGCTCGGAGATGTGTATAAGAGACAG | 195 |
| 45 | CAAGCAGAAGACGGCATACGAGATGACTGTAGGTCTCGTGGGCTCGGAGATGTGTATAAGAGACAG | 196 |
| 46 | CAAGCAGAAGACGGCATACGAGATTCAGTACAGTCTCGTGGGCTCGGAGATGTGTATAAGAGACAG | 197 |
| 47 | CAAGCAGAAGACGGCATACGAGATCATGATAGGTCTCGTGGGCTCGGAGATGTGTATAAGAGACAG | 198 |
| 48 | CAAGCAGAAGACGGCATACGAGATGCATCTCGGTCTCGTGGGCTCGGAGATGTGTATAAGAGACAG | 199 |
| 49 | CAAGCAGAAGACGGCATACGAGATTGCACAGAGTCTCGTGGGCTCGGAGATGTGTATAAGAGACAG | 200 |
| 50 | CAAGCAGAAGACGGCATACGAGATGAGCTATAGTCTCGTGGGCTCGGAGATGTGTATAAGAGACAG | 201 |
| 51 | CAAGCAGAAGACGGCATACGAGATAGTCTGCAGTCTCGTGGGCTCGGAGATGTGTATAAGAGACAG | 202 |
| 52 | CAAGCAGAAGACGGCATACGAGATCGGTGTGAGTCTCGTGGGCTCGGAGATGTGTATAAGAGACAG | 203 |
| 53 | CAAGCAGAAGACGGCATACGAGATCTGATGTGGTCTCGTGGGCTCGGAGATGTGTATAAGAGACAG | 204 |
| 54 | CAAGCAGAAGACGGCATACGAGATGCACTCTAGTCTCGTGGGCTCGGAGATGTGTATAAGAGACAG | 205 |
| 55 | CAAGCAGAAGACGGCATACGAGATTCTGCTCAGTCTCGTGGGCTCGGAGATGTGTATAAGAGACAG | 206 |
| 56 | CAAGCAGAAGACGGCATACGAGATTGTATCTAGTCTCGTGGGCTCGGAGATGTGTATAAGAGACAG | 207 |
| 57 | CAAGCAGAAGACGGCATACGAGATACAGTGAGGTCTCGTGGGCTCGGAGATGTGTATAAGAGACAG | 208 |
| 58 | CAAGCAGAAGACGGCATACGAGATATGCGATAGTCTCGTGGGCTCGGAGATGTGTATAAGAGACAG | 209 |
| 59 | CAAGCAGAAGACGGCATACGAGATGAGACATGGTCTCGTGGGCTCGGAGATGTGTATAAGAGACAG | 210 |
| 60 | CAAGCAGAAGACGGCATACGAGATGTCATGTGGTCTCGTGGGCTCGGAGATGTGTATAAGAGACAG | 211 |
| 61 | CAAGCAGAAGACGGCATACGAGATTCATGATAGTCTCGTGGGCTCGGAGATGTGTATAAGAGACAG | 212 |
| 62 | CAAGCAGAAGACGGCATACGAGATGTCATCTCGTCTCGTGGGCTCGGAGATGTGTATAAGAGACAG | 213 |
| 63 | CAAGCAGAAGACGGCATACGAGATTATCTCTAGTCTCGTGGGCTCGGAGATGTGTATAAGAGACAG | 214 |
| 64 | CAAGCAGAAGACGGCATACGAGATCTGATATAGTCTCGTGGGCTCGGAGATGTGTATAAGAGACAG | 215 |
| 65 | CAAGCAGAAGACGGCATACGAGATTACGCATGGTCTCGTGGGCTCGGAGATGTGTATAAGAGACAG | 216 |
| 66 | CAAGCAGAAGACGGCATACGAGATCGTGAGTGGTCTCGTGGGCTCGGAGATGTGTATAAGAGACAG | 217 |
| 67 | CAAGCAGAAGACGGCATACGAGATGACACATGGTCTCGTGGGCTCGGAGATGTGTATAAGAGACAG | 218 |
| 68 | CAAGCAGAAGACGGCATACGAGATACATGACGGTCTCGTGGGCTCGGAGATGTGTATAAGAGACAG | 219 |
| 69 | CAAGCAGAAGACGGCATACGAGATGCGTCTAGGTCTCGTGGGCTCGGAGATGTGTATAAGAGACAG | 220 |
| 70 | CAAGCAGAAGACGGCATACGAGATTCACGCTGGTCTCGTGGGCTCGGAGATGTGTATAAGAGACAG | 221 |
| 71 | CAAGCAGAAGACGGCATACGAGATTCATGTACGTCTCGTGGGCTCGGAGATGTGTATAAGAGACAG | 222 |
| 72 | CAAGCAGAAGACGGCATACGAGATTAGTGACGGTCTCGTGGGCTCGGAGATGTGTATAAGAGACAG | 223 |
| 73 | CAAGCAGAAGACGGCATACGAGATCACGATAGGTCTCGTGGGCTCGGAGATGTGTATAAGAGACAG | 224 |
| 74 | CAAGCAGAAGACGGCATACGAGATACACACTGGTCTCGTGGGCTCGGAGATGTGTATAAGAGACAG | 225 |
| 75 | CAAGCAGAAGACGGCATACGAGATAGCATCACGTCTCGTGGGCTCGGAGATGTGTATAAGAGACAG | 226 |
| 76 | CAAGCAGAAGACGGCATACGAGATTAGTCGTAGTCTCGTGGGCTCGGAGATGTGTATAAGAGACAG | 227 |
| 77 | CAAGCAGAAGACGGCATACGAGATGCATCGCGGTCTCGTGGGCTCGGAGATGTGTATAAGAGACAG | 228 |
| 78 | CAAGCAGAAGACGGCATACGAGATATCATGTCGTCTCGTGGGCTCGGAGATGTGTATAAGAGACAG | 229 |
| 79 | CAAGCAGAAGACGGCATACGAGATGTACTCTAGTCTCGTGGGCTCGGAGATGTGTATAAGAGACAG | 230 |
| 80 | CAAGCAGAAGACGGCATACGAGATAGTGCATAGTCTCGTGGGCTCGGAGATGTGTATAAGAGACAG | 231 |
| 81 | CAAGCAGAAGACGGCATACGAGATCGCATCAGGTCTCGTGGGCTCGGAGATGTGTATAAGAGACAG | 232 |
| 82 | CAAGCAGAAGACGGCATACGAGATCGCTATAGGTCTCGTGGGCTCGGAGATGTGTATAAGAGACAG | 233 |
| 83 | CAAGCAGAAGACGGCATACGAGATGTCGATAGGTCTCGTGGGCTCGGAGATGTGTATAAGAGACAG | 234 |
| 84 | CAAGCAGAAGACGGCATACGAGATACACAGTAGTCTCGTGGGCTCGGAGATGTGTATAAGAGACAG | 235 |
| 85 | CAAGCAGAAGACGGCATACGAGATCAGATGTAGTCTCGTGGGCTCGGAGATGTGTATAAGAGACAG | 236 |
| 86 | CAAGCAGAAGACGGCATACGAGATCTCTACAGGTCTCGTGGGCTCGGAGATGTGTATAAGAGACAG | 237 |
| 87 | CAAGCAGAAGACGGCATACGAGATGTCACTAGGTCTCGTGGGCTCGGAGATGTGTATAAGAGACAG | 238 |
| 88 | CAAGCAGAAGACGGCATACGAGATTGTACTAGGTCTCGTGGGCTCGGAGATGTGTATAAGAGACAG | 239 |
| 89 | CAAGCAGAAGACGGCATACGAGATACGCTATGGTCTCGTGGGCTCGGAGATGTGTATAAGAGACAG | 240 |
| 90 | CAAGCAGAAGACGGCATACGAGATATGTATAGGTCTCGTGGGCTCGGAGATGTGTATAAGAGACAG | 241 |
| 91 | CAAGCAGAAGACGGCATACGAGATTGTGACAGGTCTCGTGGGCTCGGAGATGTGTATAAGAGACAG | 242 |

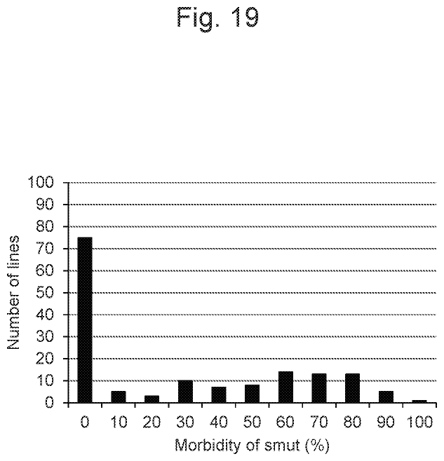

Among them, 2,936 genotypes of KY09-6092 and 1,877 genotypes of KY08-129 were obtained. The genetic map data comprising 117 linkage groups derived from “KY09-6092” and the genetic map data comprising 123 linkage groups derived from “KY08-129” were obtained. In addition, the smut resistance test data of the 154 progeny lines resulting from crossbreeding between “KY09-6092” and “KY08-129” were obtained under the same conditions as in Example 1, and the results of calculation of the morbidity of smut were shown in FIG. 19. Based on the genetic map data and the smut resistance test data, QTL analysis was carried out by the composite interval mapping (CIM) method using the QTL Cartographer gene analysis software (statgen.ncsu.edu/qticart/cartographer.html). The LOD threshold was determined to be 2.5.

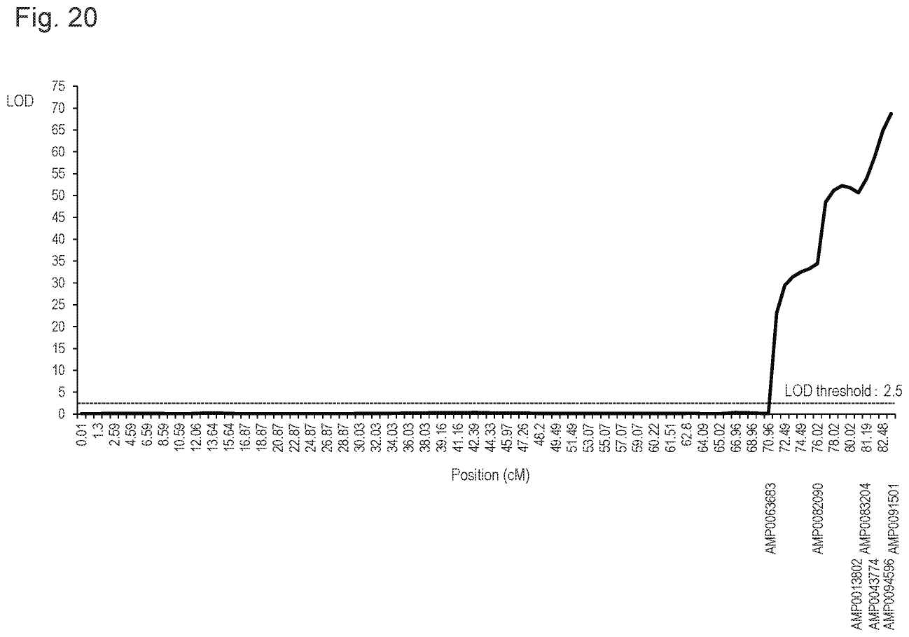

As a result, the presence of QTL linked to sugarcane smut resistance was confirmed in a region of approximately 12.27 cM including the markers AMP0063683 to AMP0091501 in the 8th linkage group of “KY09-6092” (Table 13 and FIG. 20). When the value indicating the effect is negative, QTL is linked to a trait of improving smut resistance.

TABLE 13

| Linkage | Position | Range | | LOD | Effects |
|---|---|---|---|---|---|
| group | (cM) | (cM) | Adjacent marker | value | (%) |
| 8 | 83.8 | 12.27 | AMP0063683-AMP0091501 | 68.7 | −54.2 |

As shown in Table 13 and FIG. 20, the range including the markers AMP0063683 to AMP0091501 in the 8th linkage group observed in this example was found to exhibit a significantly higher LOD value and significantly improved effects, compared with those described in WO 2012/147635. The markers included in the QTL region linked to sugarcane smut resistance confirmed in this example (i.e., AMP0063683, AMP0082090, AMP0013802, AMP0083204, AMP0043774, AMP0094596, and AMP0091501) were selected as selection markers (Table 14).

TABLE 14

| | Linkage | | | SEQ ID |
|---|---|---|---|---|
| Varity | group | Marker | PCR amplicon sequence | NO: |
| Iriomote | 8 | AMP0063683 | Read 1 (5′→3′): | SEQ ID |
| 8 | | | GATCCCGGAGGAAGTTCAGGACCGGCCAGAGCAG | NOs: 144, |
| | | | CCAGAGGTACCAACCGGCGACGAAGCTCCTGCCGA | 145 |
| | | | AGATCTACCCGAGTGTCCTGACCACCGGCCC (SEQ | |
| | | | ID NO: 144) | |
| | | | Read 2 (5′→3′): | |
| | | | TACAATGCAGCAAGTGTTTTCATATAACTCCTAGAAC | |
| | | | TTAATGCAATAAACAATTAAAAGTACTCAAGATAGAT | |
| | | | TTATAAAAATGATGGGTTCAGCATGC (SEQ ID NO: | |
| | | | 145) | |
| Iriomote | 8 | AMP0082090 | GCGGAAAATAGACCAATGTATTAGGCTATCCATTGC | SEQ ID |
| 8 | | | CGATATGCAAAAGAATGAATCCATAATGATAGCCGC | NO: 146 |
| | | | CGCTCACTTCTGGTCAGACACCACAAATACCTTTAT | |
| | | | GTTTGGTCATGGTCCATCCGCCCCTACTCTTGCCGA | |
| | | | TGTATACATGCTCACCGACTTAGATATCTCA | |
| Iriomote | 8 | AMP0013802 | CAGGAACTTGCAAACAAGCTCGGGTGCACAGATCC | SEQ ID |
| 8 | | | AAATGATTTGGTCGGTTGATGTGGACTCCGATGTGG | NO: 147 |
| | | | ACTCAAATGC | |
| Iriomote | 8 | AMP0083204 | GCGTGGCAGAGGATGGGGGCACGACAGGGATGGC | SEQ ID |
| 8 | | | ACCGTGGCGAGGGGAGGAGCACGGTGTGGCAGGG | NO: 148 |
| | | | AAAGGAGCGCGGCGAGCTTGACGCCGGGGGCGCA | |
| | | | ACCAGGATGAGGATTGAGTCACGGATGGTTGGAC | |
| Iriomote | 8 | AMP0043774 | GAAAACAAGCTCTACCACTGGCACAACACGGGCTC | SEQ ID |
| 8 | | | ACAGGTCACAGCTCTGCTTGCTCAGTGGTACTACGA | NO: 149 |
| | | | GTGCTAGCACGGGAGCGCTGTTGGACGGATCGCCA | |
| | | | GCATGAAATGCCCACCGCGCACGTACTGC | |
| Iriomote | 8 | AMP0094596 | TACCCCACCAAAAAGCCTATCATGATCCTTGGTGTC | SEQ ID |
| 8 | | | ATTTTATTTTTGGTTCTGTCAGGTAAGTCTAGCTGAG | NO: 150 |
| | | | TACCTTCTCATACTCAGGGCTTTGTTCCCACTTGTTA | |
| | | | CAGATGGACAGATGTACTATGGTTATTGTATCA | |
| Iriomote | 8 | AMP0091501 | TAACCCACCAAAGATAGCCTTGCATGCTCCTTAGTG | SEQ ID |
| 8 | | | TCGCTTATTTTGGTTTAAGACGGGTAAGTCTAGCTG | NO: 151 |
| | | | AGTACCTTCTCGTACTCAGGGCGTTGTTCTCATTGT | |
| | | | TGTTGCAGATGGTTAGATGTACTATGGATATTGCGT | |
| | | | CA | |

Among the markers shown in Table 14, the PCR product of AMP0063683 is 200 bp or larger. Thus, AMP0063683 is defined as a nucleic acid region comprising Read 1 (SEQ ID NO: 144) and Read 2 (SEQ ID NO: 145) with the nucleotide sequences being determined using the next-generation sequencer at both ends.

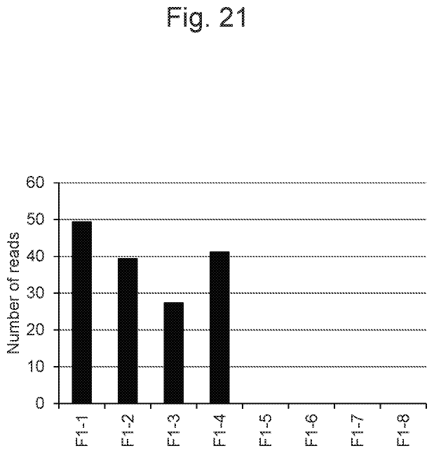

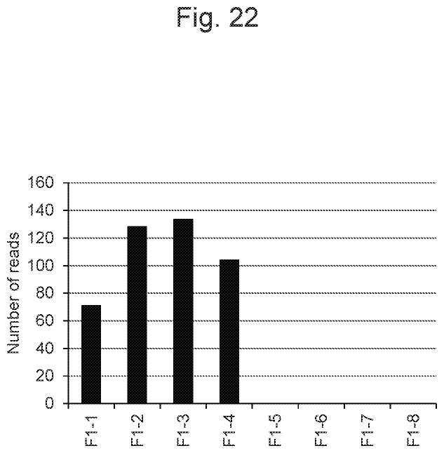

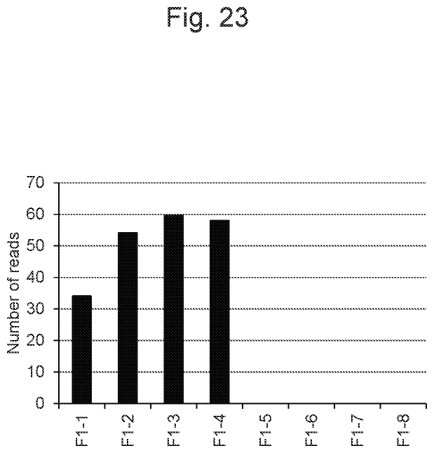

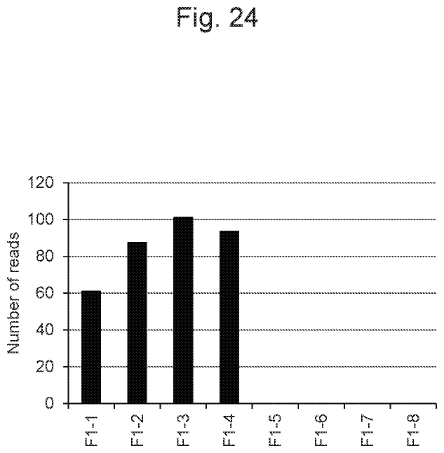

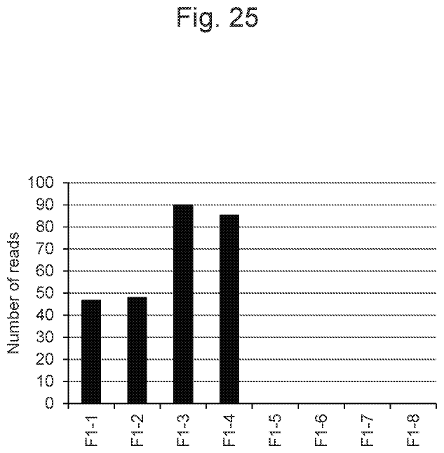

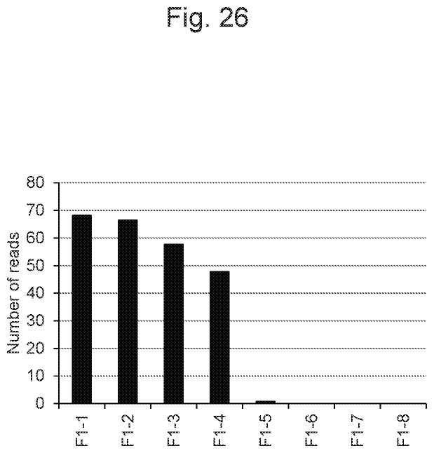

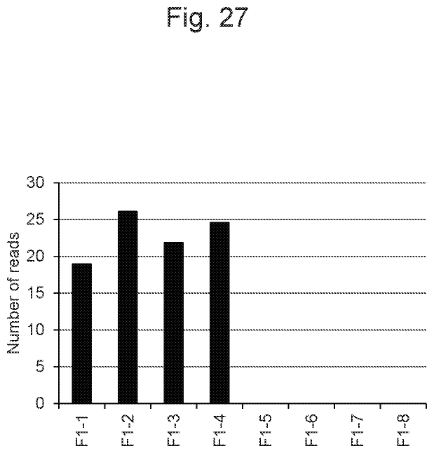

Whether or not genotypes of the markers; i.e., the line samples, have the selection markers was determined by designating the threshold of each number of reads as 10, evaluating as “present” when the number of reads is 10 or more, and evaluating as “absent” when the number of reads is less than 10 (FIGS. 21 to 27, Table 15).

TABLE 15

| Marker | F1-1 | F1-2 | F1-3 | F1-4 | F1-5 | F1-6 | F1-7 | F1-8 |
|---|---|---|---|---|---|---|---|---|
| AMP0063683 | 49.4 | 39.4 | 27.4 | 41.2 | 0 | 0 | 0 | 0 |
| AMP0082090 | 70.9 | 128.1 | 133.4 | 104.1 | 0 | 0 | 0 | 0 |
| AMP0013802 | 34.1 | 54.2 | 59.6 | 58.1 | 0 | 0 | 0 | 0 |
| AMP0083204 | 61 | 87.5 | 101.2 | 93.7 | 0 | 0 | 0 | 0 |
| AMP0043774 | 46.7 | 48 | 89.9 | 85.3 | 0 | 0 | 0 | 0 |
| AMP0094596 | 68.2 | 66.5 | 57.7 | 47.8 | 0.8 | 0 | 0 | 0 |
| AMP0091501 | 19 | 26.1 | 21.9 | 24.6 | 0 | 0 | 0 | 0 |
| Incidence of | 0 | 0 | 0 | 0 | 70.28 | 18.8 | 61.74 | 52.63 |
| smut % | | | | | | | | |

As shown in Table 15 and FIGS. 21 to 27, line samples with the low morbidity of smut have a significantly larger number of markers than line samples with the high morbidity of smut. This demonstrates that continuous nucleic acid regions selected from a region of approximately 12.27 cM comprising AMP0063683, AMP0082090, AMP0013802, AMP0083204, AMP0043774, AMP0094596, and AMP0091501 can be used as the markers associated with sugarcane smut resistance.

### Example 4

In this example, the nucleotide sequence of the region of approximately 8.4 cM comprising the marker associated with sugarcane smut resistance derived from JW90 identified in Example 1 was compared with the nucleotide sequence of the region of approximately 12.27 cM comprising the marker associated with sugarcane smut resistance derived from Iriomote 8 identified in Example 3. As a result, the adjacent region comprising AMP0121265 located at 0 cM among the markers associated with sugarcane smut resistance derived from JW90 identified in Example 1 and the adjacent region comprising AMP0091501 located at 83.76 cM among the markers associated with sugarcane smut resistance derived from Iriomote 8 identified in Example 3 were found to comprise a plurality of markers having the identical nucleotide sequence.

Table 16 summarizes markers included in the adjacent region comprising AMP0121265 located at 0 cM among the markers associated with sugarcane smut resistance derived from JW90 identified in Example 1. Table 17 summarizes markers included in the adjacent region comprising AMP0091501 located at 83.76 cM among the markers associated with sugarcane smut resistance derived from Iriomote 8 identified in Example 3. In Tables 16 and 17, the column indicating the nucleotide sequence information of markers shows the nucleotide sequences of contig sequences when the contig sequences can be led from a pair of read data obtained via analysis using a next-generation sequencer, or a pair of read sequences (the read sequence 1 and the read sequence 2) when the contig sequence cannot be led. Specifically, the markers shown in Tables 16 and 17 can be defined by the nucleotide sequences of the contig sequences or the nucleotide sequences of the read sequence 1 and the read sequence 2.

Among the markers shown in Table 16, concerning the markers having the identical nucleotide sequence with the markers included in the adjacent region including AMP0179276 shown in Table 17, the column indicating “Presence or absence of DNA marker derived from Iriomote 8 (): Iriomote 8 marker ID” shows “Present” and ID of the marker derived from Iriomote 8 having the identical nucleotide sequence. Similarly, among the markers shown in Table 17, concerning the markers having the identical nucleotide sequence with the markers included in the adjacent region including AMP0121265 shown in Table 16, the column indicating “Presence or absence of DNA marker derived from JW90 (): JW90 marker ID” shows “Present” and ID of the marker derived from JW90 having the identical nucleotide sequence.

TABLE 16

| | | | | Presence of absence |
|---|---|---|---|---|
| | | | | of DNA marker |
| | | | | derived from Iriomote |
| Marker ID | Origin | Nucleotide sequence information | SEQ ID NO: | 8 (): Marker ID |
| AMP0129420 | JW90 | ACAGGGAAGTGAAAGAGAAGTGGCCATTGAAA | SEQ ID NO: | — |
| | | AACCTAATATTGTTACGGAAGTGCCTCGCGTCG | 243 | |
| | | GCGTCGAAGCTCGTCCAAAGCCTCATGAGCTG | | |
| | | GTCTGGTATGACTGGCGCATTCAGCTCGA | | |
| AMP0010186 | JW90 | CAAGCGCAGGAAAAGGAAACGCTGCCCGCAGT | SEQ ID NO: | — |
| | | TCCTCACCCGGCTCCCCCGTTGTCGCCCTCAG | 244 | |
| | | CCGCTTCCGCCATCCTCGCTGTCAC | | |
| AMP0010846 | JW90 | CAATAAAATGGGAACATGATGGAAGGACAGGT | SEQ ID NO: | — |
| | | TACCCGATGCATTATGAGCATACAGAACGGCCT | 245 | |
| | | ACAAAACACCGATTGGAATGTCACCATATCAAC | | |
| | | TTATCTATGGAAGA | | |
| AMP0015478 | JW90 | CAGACAGATAGCCACGAGACGAGGACTCACAG | SEQ ID NO: | — |
| | | AGCTTTCGGTGAGCCTCCATAGTCCAGAGATA | 246 | |
| | | GTCTCTGTTGGTTCTTTTTCTTTCCTTTTTCGAC | | |
| | | GAGATCTTTTTTTCTTGGTCCCACACGAACTGG | | |
| | | CGTTGTGCTTCTATTATGCTGTCAC | | |
| AMP0019992 | JW90 | CATCATGGTGCGAATCTTCGAGTTCAGACCCCT | SEQ ID NO: | — |
| | | CATAAAACAAGCTTGCTTCTTTCGATCTGAGTT | 247 | |
| | | GATTTGGTCAGCTGCATACTATGATAAATGATT | | |
| | | GAACCTACCCACATATTGCATC | | |
| AMP0020554 | JW90 | CATGAAAGCTCACAAACCCGCTCGAGCTCTGG | SEQ ID NO: | Present |
| | | GTCGATGGCTCATCCCATTTGCCTCGAGAATG | 248 | (AMP0016471) |
| | | CCCCTCCTCCGCTGCGCAGGTGCCGCCTCTCC | | |
| | | TTACATCACGCGTCACCATGCCATGCCCGC | | |
| AMP0022987 | JW90 | Read 1 (5′→3′): | SEQ ID NOs: | — |
| | | CTAATGCTTGGTAGCTCATACATGAACCTTATA | 249, 250 | |
| | | GTCGTCCTGCATTTAACAACTGTCAAGGACAGG | | |
| | | TTCGAGTGCACATGTATAATGCTATCAACGTTT | | |
| | | T (SEQ ID NO: 249) | | |
| | | Read 2 (5′→3′): | | |
| | | TAGCCACATAACTTTCCCACAATGTAAAGGTAT | | |
| | | TTTCAATAAAGAATCTAGGAGTACTAATTCTATG | | |
| | | TTTTCAATCTATACAATCATTGGCATATTGATG | | |
| | | (SEQ ID NO: 250) | | |
| AMP0130766 | JW90 | CTCGAAAATCTCTAACAAACTCATTCCAAGTAAT | SEQ ID NO: | — |
| | | AGGAGGAGCATTGTTAGGACGTGCAGCTTGAT | 251 | |
| | | ACGACTCCCACCAAGTCTGTGTTGTCCCCTGTA | | |
| | | ACTGACCAGTTGCATATAACACCTTTTCCATAT | | |
| | | CATTGCATTGAGCGATGTTCA | | |
| AMP0030465 | JW90 | Read 1 (5′→3′): | SEQ ID NOs: | — |
| | | CTCGAGCGATGTCCGATAGGAACAGGGGAAGT | 252, 253 | |
| | | TGTCGAATGATGACCCGGTCGTCGGTGGCCCC | | |
| | | ACCCAACTGACTAGCCAAGCGGAAGTCAGCGA | | |
| | | GCCA (SEQ ID NO: 252) | | |
| | | Read 2 (5′→3′): | | |
| | | TCGGAGGATCGAAGCCCTTCACCCGAGCCACC | | |
| | | AGGCCCTCGGGTCTTTAGCAAAGCCATCCGCA | | |
| | | AAACGTTACTCCCAGCTCGGTTCCTCCCCCATG | | |
| | | ACT (SEQ ID NO: 253) | | |
| AMP0031132 | JW90 | Read 1 (5′→3′): | SEQ ID NOs: | — |
| | | CTCGAGCGGTGTCAGAGAGGAACAGGGGGAG | 254, 255 | |
| | | CTGCCGGATGATGTTCCGGTCGTCGGTGGCCC | | |
| | | CGCCCAGCTGACACGCCAAGCGGAAATCGGC | | |
| | | GAGCCA (SEQ ID NO: 254) | | |
| | | Read 2 (5′→3′): | | |
| | | TCGGAGGATCGTAGCCCTTCACCCGAGCCTCC | | |
| | | GGGCCCTCGGGTCTTCAGTAAGGCTATTCGTG | | |
| | | AAATGATACTCCCGGCGCGGTTTCGCCCCCCC | | |
| | | CCCA (SEQ ID NO: 265) | | |
| AMP0033330 | JW90 | Read 1 (5′→3′): | SEQ ID NOs: | — |
| | | CTCGAGCGGTGTCGAAGAGGAACAGAGGGAG | 256, 257 | |
| | | CTGTCGGATGATGACCTGGTCGTCGGTGGCCC | | |
| | | CACCCAGCTGACATGCCAAGCGGAAATCGGCG | | |
| | | AGCCA (SEQ ID NO: 256) | | |
| | | Read 2 (5′→3′): | | |
| | | TCGGAGGATCGCAGCCCATCACCCGAGCCTCC | | |
| | | AGGCCCTCGGGTCTTCAGTAAGGCTATCCGCG | | |
| | | AAACTATACTCCCGGCGCAGTTTCGCCCTCCC | | |
| | | ACGA (SEQ ID NO: 257) | | |
| AMP0036149 | JW90 | Read 1 (5′→3′): | SEQ ID NOs: | — |
| | | CTCGAGCGGTGTCGGAGAGGAACAGGGGGAG | 258, 259 | |
| | | CTGTCGGATGATGACCCGGTCGTCGGTGGCCC | | |
| | | CACCCAGCTGACACGCCAAGCGGAAATCGGCG | | |
| | | AGCCA (SEQ ID NO: 258) | | |
| | | Read 2 (5′→3′): | | |
| | | TCGGAGGATCGTAGCCCTTCACCCGAGCCTCC | | |
| | | GGGCCCTCGGGTCTTCAGTAAGGCTATCCGCG | | |
| | | AAATAATACTCCCGGCGCGGTTTCGCCCTCCC | | |
| | | ACGA (SEQ ID NO: 259) | | |
| AMP0036465 | JW90 | Read 1 (5′→3′): | SEQ ID NOs: | — |
| | | CTCGAGCGGTGTCGGAGAGGAACAGGGGGAG | 260, 261 | |
| | | CTGTCGGATGATGACCCGGTCGTCGGTGGCCC | | |
| | | CGCCCAGCTGACATGCCAAGCGGAAATCGGCG | | |
| | | AGCCA (SEQ ID NO: 260) | | |
| | | Read 2 (5′→3′): | | |
| | | TCGGAGGATCGAAGCCCTTCACCCGAGCCTCC | | |
| | | GGGCCCTCGGGTCTTCAGCAAGGCTATCCGCG | | |
| | | AAACGATACTCCTGGCGCGGTTTCGCCCTCCC | | |
| | | ACGA (SEQ ID NO: 261) | | |
| AMP0039231 | JW90 | Read 1 (5′→3′): | SEQ ID NOs: | — |
| | | CTCGAGCGGTGTTAGAGAGGAACAGGGGGAG | 262, 263 | |
| | | CTGTCGGATGATGACCCGGTCGTCGGTGGCCC | | |
| | | CACCCAGCTGACATGCCAAGCGGAAATCGGCA | | |
| | | AGCCA (SEQ ID NO: 262) | | |
| | | Read 2 (5′→3′): | | |
| | | TCGGAGGATCGTAGCCCTTCACCCGAGCCTCC | | |
| | | GGGCCCTCGGGTCTTCAGTAAGGCTATCCGCG | | |
| | | AAACGATACTCCCGGCGCGGTTTCGCCCTCCC | | |
| | | ACGA (SEQ ID NO: 263) | | |
| AMP0040432 | JW90 | Read 1 (5′→3′): | SEQ ID NOs: | — |
| | | CTCGGGCGGTATCGGAGAGGAATAGGGGGAG | 264, 265: | |
| | | TTGTCGGATGATGACCCGGTCGTCGGTGGCCG | | |
| | | CGCCTAGCTAACACGCCAAGCAGAAATCGGCG | | |
| | | AGCCA (SEQ ID NO: 264) | | |
| | | Read 2 (5′→3′): | | |
| | | TCAGAAGATCGTAGCCCTTCACCCGAGCCTCC | | |
| | | GGGCTCTCGGGTCTTTAGTAAGGCTATCCGCG | | |
| | | AGACGGTGCTCCCGGCTCGGTTTCGCCCTCCC | | |
| | | ACGA (SEQ ID NO: 265) | | |
| AMP0041203 | JW90 | Read 1 (5′→3′): | SEQ ID NOs: | — |
| | | CTCGGGCGGTGTCGGAGAGGAACAGGGGGAG | 266, 267 | |
| | | TTATCGAATGATGACCCGGTCGTCGGTGGCCC | | |
| | | CGCCCAGCTGACACGCCAAGCGGAAATCGGTG | | |
| | | AGCCA (SEQ ID NO: 266) | | |
| | | Read 2 (5′→3′): | | |
| | | TCGGAAGATCGTAGTCCTTCACCCGAGCCTCC | | |
| | | GGGCCCTCTGGTCTTCTGTAAAGCTATCCGTG | | |
| | | AAATGGTGCTCCCGGCTCAGTTTCGCCCTCCC | | |
| | | ACGA (SEQ ID NO: 267) | | |
| AMP0041520 | JW90 | Read 1 (5′→3′); | SEQ ID NOs: | — |
| | | CTCGGGCGGTGTCGGAGAGGAACAGGGGGAG | 268, 269 | |
| | | TTGTCGAATGATAACCCGGTCGTCGGTGGCCC | | |
| | | CACCCAGCTGACATGCCAAGCGGAAATCAGGA | | |
| | | AGCCA (SEQ ID NO: 268) | | |
| | | Read 2 (5′→3′): | | |
| | | TCGAAAGATCGTAGTCCTTCACCCGAGCCTCC | | |
| | | GGGCCCTCGGGTCTTCAGTAAGGCTATCCGTG | | |
| | | AAACGGTGCTCCCATCTCGGTTTCGCCCTCCC | | |
| | | ACGA (SEQ ID NO: 269) | | |
| AMP0045000 | JW90 | CTGAGTCTAGTAATAAGACTTTGATCAAGCTTA | SEQ ID NO: | Present |
| | | TCAAGAAGAAAATTGAGGAAAATTCAAGGAGGT | 270 | (AMP0036426) |
| | | GGCATGAAGTTTTGTCTGAAGCTCTATGGGCAC | | |
| | | ATCGTATTTCAAAACATGGTGCTACCAAAGTTA | | |
| | | TTCCTTTTAAGCTAGTATATGACCAAGAGGCCG | | |
| | | TGTTA | | |
| AMP0057239 | JW90 | GAAGCTACTGTTGGTTGGGAGCTAATCGGAAT | SEQ ID NO: | Present |
| | | GACTATTTATCATTTTTTGAAAATAAAAAGGGAT | 271 | (AMP0046626) |
| | | GACTGATACTTATGCACA | | |
| AMP0059273 | JW90 | GACAATGCATCACGCCGATGTAGCCGAGATCA | SEQ ID NO: | — |
| | | TCCCTTCGCTGCCCCTGGAGATCAGGTGGCCA | 272 | |
| | | CCGTTCCGTCTCCGCCATTAC | | |
| AMP0062853 | JW90 | GACCACGCGGGCGACATCGACACCAGCAAGA | SEQ ID NO: | Present |
| | | GCACGAGCGGGATTCTCTTCTTCCTCGGCAGG | 273 | (AMP0052709) |
| | | TGCCTCGTTAGCTGGCAGTCGGTCAAGCAGCA | | |
| | | GGTGGTGGCCCTGTCCAGCTGCGAGGCCGAG | | |
| | | TACATAGCGGCTTCCACCGCTTCGACTTAGGC | | |
| | | GC | | |
| AMP0064313 | JW90 | GACCTGGCAGGCGACGTGAATGACTGAAAGAG | SEQ ID NO: | — |
| | | CACCAGCGGGCTGATCTTCTTCCTGGCAGGAG | 274 | |
| | | GCCCGATTGCTTGGCAGTCGGCAAAACAGAGG | | |
| | | GTGGTTGC | | |
| AMP0132654 | JW90 | Read 1 (5′→3′): | SEQ ID NOs: | — |
| | | GAGATGAGAGGAGCGAGGAAAGGAGAGGAAG | 275, 276 | |
| | | GGAGGTGAGGTGTTGTAGTGTGGATACAGCTA | | |
| | | ACAGCTTACATGGAGGTGGAGTATGGGGCCTG | | |
| | | GTTTA (SEQ ID NO: 275) | | |
| | | Read 2 (5′→3′): | | |
| | | GCGCAGGAAAGAGGTCGTGGACAAATCCAGG | | |
| | | GCGTGTTTAGTTGGGCTGATTTTGGACGTCCAA | | |
| | | AATCTGCACAGTGTAAGATTCCATACTGTAGCA | | |
| | | TTT (SEQ ID NO: 276) | | |
| AMP0070602 | JW90 | Read 1 (5′→3′): | SEQ ID NOs: | — |
| | | GAGCGAGACGAGAGACAAACAAAAAGGTGGTG | 277, 278 | |
| | | ACGATAAGAGGCGCCACGCACTAAACCCGCAC | | |
| | | CATGGTCATGGAGTAGCGTGTAAGGAACAAGT | | |
| | | AGCA (SEQ ID NO: 277) | | |
| | | Read 2 (5′→3′): | | |
| | | GCGAGAGGTGGTGGTACACGCCTACCTAGGTG | | |
| | | GTGGGCTCGTCGTCGTCAGACACCAGGCGTCA | | |
| | | CAGGCCGTCGTCCGTTTCTGCCGCCCCATGCT | | |
| | | CGCT (SEQ ID NO: 278) | | |
| AMP0133317 | JW90 | Read 1 (5′→3′): | SEQ ID NOs: | — |
| | | GCACGACATGGTATGGCAGGGGACAAAGACG | 279, 280 | |
| | | GAGATGGGGTGCGCCGTGGGGGATGGCTTCA | | |
| | | GCGACATGCCAGAGTGGGCCACGGCGCGACA | | |
| | | GAGTGGG (SEQ ID NO: 279) | | |
| | | Read 2 (5′→3′): | | |
| | | GCTGCTCCCTTCTCCGGCGCGAGGCTCCTCGT | | |
| | | CGTGGCCTTCTCTAGCGCGGTGGCACCGCATC | | |
| | | CGCTCCCATCCCGCGCTGCTCCACACTCCGCG | | |
| | | CGCG (SEQ ID NO: 280) | | |
| AMP0090028 | JW90 | Read 1 (5′→3′): | SEQ ID NOs: | — |
| | | GCCACCGGCCTCGTGGTCGTGAGACCGAGGA | 281, 282 | |
| | | GACCGGTACCTGGGATGGGCTGGTAGTCCTCA | | |
| | | TCTGTGCTGTCAGAAGTCACATCCTCAGGTATA | | |
| | | TCAG (SEQ ID NO: 281) | | |
| | | Read 2 (5′→3′): | | |
| | | GTGAGCGCCCAGACGTTCCTGAGACAGCAGCT | | |
| | | GAGCAGGATGCCAGAGTTGACGAGCTTGCTGA | | |
| | | GGCAGAGCTTGCGGACCTTGAGGCTCAGACTG | | |
| | | CTGA (SEQ ID NO: 282) | | |
| AMP0100461 | JW90 | GCGGTTGTGTGTTGTGGCCGGGCCATTGGCG | SEQ ID NO: | — |
| | | GATGAGCGAGCGAGTGCAACCAGAGGCGAAC | 283 | |
| | | GTACCCAACCCAGCGACAGCAAGTCAGCAACA | | |
| | | TATATTCTCTCACTCGATCACGTCCCCACGGGC | | |
| | | CTA | | |
| AMP0134140 | JW90 | GCTAAGAACATTCGGTGTCTGCAATGTGAGAA | SEQ ID NO: | — |
| | | GTGAACACTCTAGTAACTTAAGAAATGCTATCA | 284 | |
| | | ACAAAATGAGCCATCTTGTTCATCTAGACATTG | | |
| | | CGGCTCTAGGGGAGAGCGAAGTGTTGCAGCTA | | |
| | | GAGGGACTTCATTTGCCTCCA | | |
| AMP0103131 | JW90 | Read 1 (5′→3′): | SEQ ID NOs: | — |
| | | GGCAGAGGGAGCCAAGCAACTAATCAAACTCA | 285, 286 | |
| | | AAGCGCCTCCTACCAACCGAAAAGGCGAGAAA | | |
| | | AAGCTAAGCCAAGGTGGGGATCGGAAGAATTA | | |
| | | TCCA (SEQ ID NO: 285) | | |
| | | Read 2 (5′→3′): | | |
| | | TAGCAAGTGCGGCTGCTATCTTGAGAGGTTGG | | |
| | | CGCTGTGGTGCTAGGCTTTTTGCCGTGCTGGG | | |
| | | TGCCTGGGTGGTGGAGATGGATGGATGTATAT | | |
| | | ATAG (SEQ ID NO: 286) | | |
| AMP0105230 | JW90 | GTATGTCACCTTTGAGGGTTTTGACTTTTAATG | SEQ ID NO: | — |
| | | AATTTGCAACTATGGCACCCAATTTTTGCAGCT | 287 | |
| | | GTAGTGTTGCTTCCATCATCAGTCAGGTCTACA | | |
| | | CCTACCAAATAATTCTAGTGTTCCATGGCTCCA | | |
| | | ATGAAATGTGGATTGAAAGTTAATCTTAATTGCA | | |
| | | TTA | | |
| AMP0107179 | JW90 | GTGAGCGCCCAGATGTTCCTGAGACAGCAGCT | SEQ ID NO: | — |
| | | GAGCAGGACGCTACAGTTGACGAGCTTGCTGA | 288 | |
| | | GGCAGAGCTCGCAGACCTTGAGACTCAGACTG | | |
| | | CTGACATACCTGAGGATGTCACTTCAGATAGCA | | |
| | | CAGATGAGGACTACCAGCCCATTCCCAGGTA | | |
| AMP0107330 | JW90 | GTGAGCGCCCAGATGTTCCTGAGACAGCAGCT | SEQ ID NO: | — |
| | | GAGCAGGACGCTACAGTTGACGAGCTTGCTGA | 289 | |
| | | GGCAGAGCTCGCGGATCTTGAGGCTCAGACTG | | |
| | | CTGACATACCTGAGGATGCCACTTCTGATAGCA | | |
| | | CAGATGAGGACTACCAGCCCATTCCCAGGTA | | |
| AMP0108751 | JW90 | Read 1 (5′→3′): | SEQ ID NOs: | — |
| | | GTGGGGCGTGTGTCTCACCCAACGAAGTAGTG | 290, 291 | |
| | | GCCAAGTAAGGTAGCCAGCGGTGGGCGAGCT | | |
| | | CCTTATTTGATGACGTGGTCCAGAAAATGGTTC | | |
| | | TCTT (SEQ ID NO: 290) | | |
| | | Read 2 (5′→3′): | | |
| | | TCATGTCCAAGTACTCGCAAGCTGATGCTTGG | | |
| | | GGGCTACAACCACTGGGGTCTCCTGAGCGCAA | | |
| | | ATTGTCAGGATCGCGCGCTGATTCTACCACGC | | |
| | | GGCC (SEQ ID NO: 291) | | |
| AMP0111891 | JW90 | TAACCCACCAAAGATAGCCTTGCATGCTCCTTA | SEQ ID NO: | Present |
| | | GTGTCGCTTATTTTGGTTTAAGACGGGTAAGTC | 292 | (AMP0091501) |
| | | TAGCTGAGTACCTTCTCGTACTCAGGGCGTTGT | | |
| | | TCTCATTGTTGTTGCAGATGGTTAGATGTACTA | | |
| | | TGGATATTGCGTCA | | |
| AMP0121265 | JW90 | TAGCCCACTAAAAGAAAGCCTTGCATAACCCTT | SEQ ID NO: | — |
| | | GATGTCACTTTATTTTGGTTTAAGACAGATAAGT | 293 | |
| | | CTAGCTGAGTACCTTCTCGTACTTAGGGCGTTG | | |
| | | TTCCCATTGTTGTTGTAGATGATTAGATGTACTA | | |
| | | CGGCTATTGCGTCA | | |
| AMP0135444 | JW90 | TCAAATGAAGGCCAAGCGTCAAGCAGTGAGCA | SEQ ID NO: | — |
| | | TTCTTTCAATTTCTTGGCAACCCA | 294 | |

TABLE 17

| | | | | Presence of absence |
|---|---|---|---|---|
| | | | | of JW90DNA marker |
| Marker ID | Origin | Nucleotide sequence information | SEQ ID NO: | (): Marker ID |
| AMP0007142 | Iriomote 8 | CAAAGTCTAGTAGGTACTCTCGAGACCCTCAT | SEQ ID NO: | — |
| | | ACGCTCTGATGGCGGCGAACATAGCACTCAT | 295 | |
| | | TGCCTGGTTGTCGCTAG | | |
| AMP0008511 | Iriomote 8 | Read 1 (5′→3′): | SEQ ID NOs: | — |
| | | CAAGCACTGGGTTGATTCTGGCAACTACGGC | 296, 297 | |
| | | ATCAAATTTGTTCCTCTGGGACCGGGCTTCTT | | |
| | | GCTCCAGCACATCGAACTGTGCCTTGACCCT | | |
| | | CTGATG (SEQ ID NO: 296) | | |
| | | Read 2 (5′→3′): | | |
| | | GCGAGCGAACAAACTGACCGAGGAGCTTAAC | | |
| | | GGTAAGTATCCACTGGTCGGGTTTTCTGCTGT | | |
| | | AGCGATTGTCTTGCCTGATGGAACTTCAAAAT | | |
| | | GGATG (SEQ ID NO: 297) | | |
| AMP0016471 | Iriomote 8 | CATGAAAGCTCACAAACCCGCTCGAGCTCTG | SEQ ID NO: | Present |
| | | GGTCGATGGCTCATCCCATTTGCCTCGAGAA | 298 | (AMP0020554) |
| | | TGCCCCTCCTCCGCTGCGCAGGTGCCGCCTC | | |
| | | TCCTTACATCACGCGTCACCATGCCATGCCC | | |
| | | GC | | |
| AMP0017797 | Iriomote 8 | Read 1 (5′→3′): | SEQ ID NOs: | — |
| | | CTAAAGGAAGAAACCAACACGACAAAAAACAG | 299, 300 | |
| | | AAAAGGTACGTACCAGCGTGGAAGTTCTCAAG | | |
| | | GGTGGAGAATTCCTCGGCCTGCCGAGCCACC | | |
| | | TGCTC (SEQ ID NO: 299) | | |
| | | Read 2 (5′→3′): | | |
| | | GCGGAGCACCTTGGTGTCGGAGCAGAACGC | | |
| | | CTTGGAGTTGGCTTGGAATGCCCTGGAGTCA | | |
| | | AAGCGAAATGCCTGGTCAGAGGCGGACCAGG | | |
| | | AGGTGCTC (SEQ ID NO: 300) | | |
| AMP0033151 | Iriomote 8 | Read 1 (5′→3′): | SEQ ID NOs: | — |
| | | CTCGGGCGGTGTCGGAGAGGAACAGGGGGA | 301, 302 | |
| | | GTTGTCGAATGATAACCCGGTCGTCGGTGGC | | |
| | | CCCACCCAGCTGACATGCCAAGCGGAAATCG | | |
| | | GCAAGCCA (SEQ ID NO: 301) | | |
| | | Read 2 (5′→3′): | | |
| | | TCGAAAGATCGTAGTCCTTCACCCGAGCCTCC | | |
| | | GGGCCCTCGGGTCTTCAGTAAGGCTATCCGT | | |
| | | GAAACGGTGCTCCCGGCTCGGTTTCGCCCTC | | |
| | | CCACGA (SEQ ID NO: 302) | | |
| AMP0036426 | Iriomote 8 | CTGAGTCTAGTAATAAGACTTTGATCAAGCTT | SEQ ID NO: | Present |
| | | ATCAAGAAGAAAATTGAGGAAAATTCAAGGAG | 303 | (AMP0045000) |
| | | GTGGCATGAAGTTTTGTCTGAAGCTCTATGGG | | |
| | | CACATCGTATTTCAAAACATGGTGCTACCAAA | | |
| | | GTTATTCCTTTTAAGCTAGTATATGACCAAGA | | |
| | | GGCCGTGTTA | | |
| AMP0038963 | Iriomote 8 | Read 1 (5′→3′): | SEQ ID NOs: | — |
| | | CTGGAAGGGTGATTTAGTCTCTGATCGGAAGG | 304, 305 | |
| | | CCTAGCTAAGGAGGAACAACGCTCGCTTGCG | | |
| | | ACTCTGACCTGCCTCTCCGACCGGAAGGCCT | | |
| | | GGCCAA (SEQ ID NO: 304) | | |
| | | Read 2 (5′→3′): | | |
| | | TCGGAGTCAGAATCGAGCGTCGTTCCTCCTT | | |
| | | GGCTAGGCCTTTTGGTTGGAGAGGCGGGTCA | | |
| | | GAGTTAGAAGCGAGCATCGTTCCTCCTTGGC | | |
| | | CAGGCCT (SEQ ID NO: 305) | | |
| AMP0039429 | Iriomote 8 | CTGGCACCTCCTCCAAACTCTTTCCTTCTCTC | SEQ ID NO: | — |
| | | CTCTCTCTATTTCTAAAGACTAGATCCTAATAA | 306 | |
| | | GGACTAATCTTCTCTCGATA | | |
| AMP0090720 | Iriomote 8 | GAAGCTACTGTTGGTTGGGAGCTAATCGGAA | SEQ ID NO: | Present |
| | | TGACTATTTATCATTTTTTGAAAATAAAAAGGG | 307 | (AMP0057239) |
| | | ATGACTGATACTTATGCACA | | |
| AMP0047005 | Iriomote 8 | Read 1 (5′→3′): | SEQ ID NOs: | — |
| | | GAAGGGTGGTAGGTGGGCATCGAACGGACCT | 308, 309 | |
| | | GGTACATGGGCTTGGGAAAGCCATAGAAGTC | | |
| | | GTAGATGGTGTCGTTGCTGCCGCGGATAACG | | |
| | | TTGACCG (SEQ ID NO: 308) | | |
| | | Read 2 (5′→3′): | | |
| | | GCGGGCAGACCCAAACCAGAGACGCGGCAC | | |
| | | GCGACGCGGGAGGGCGAGGAGCAGCGGCG | | |
| | | GCGCGGCGACATCGCGAGGCCAGGGCCAGA | | |
| | | CGCAACGGCCG (SEQ ID NO: 309) | | |
| AMP0048243 | Iriomote 8 | GACACACGACACGAGAGTTTGGCCATGGCAA | SEQ ID NO: | — |
| | | CGGTCTCCTCGCTCGCTCGCTCCTCACTGGC | 310 | |
| | | GGCCGTCACACCTGGAGAGTGACCGACTGG | | |
| | | CAGTAGCTGTAGCAGCCCTCAGCCTCACGCG | | |
| | | ACGCAGCGCAGGCCTCTGGATGC | | |
| AMP0052709 | Iriomote 8 | GACCACGCGGGCGACATCGACACCAGCAAG | SEQ ID NO: | Present |
| | | AGCACGAGCGGGATTCTCTTCTTCCTCGGCA | 311 | (AMP0062853) |
| | | GGTGCCTCGTTAGCTGGCAGTCGGTCAAGCA | | |
| | | GCAGGTGGTGGCCCTGTCCAGCTGCGAGGC | | |
| | | CGAGTACATAGCGGCTTCCACCGCTTCGACT | | |
| | | TAGGCGC | | |
| AMP0054926 | Iriomote 8 | Read 1 (5′→3′): | SEQ ID NOs: | — |
| | | GACGCGGCGATCAGGCCGGAAGGAGTCGTG | 312, 313 | |
| | | GCGCCGCCGTCCGTGAGCTTACCTGATTGATT | | |
| | | TGATTGATTGTTGCATTGGTTAATTATTATTAGT | | |
| | | TACT (SEQ ID NO: 312) | | |
| | | Read 2 (5′→3′): | | |
| | | GAGAATGCCGGTGAGCAGGATCGCCGTGGG | | |
| | | CGCTCCGGGCGAGCTGTCCCACCCCGACAC | | |
| | | CGCCAAGGCCGCCGTCGCCGAGTTCATCTCC | | |
| | | ATGCTCATC (SEQ ID NO: 313) | | |
| AMP0059174 | Iriomote 8 | GAGCGGCGGCGACGGCGGAAATGGAGAGGG | SEQ ID NO: | — |
| | | CGGTGGGGGGAAAGCGGCCTAGGGTTCGAG | 314 | |
| | | GGGAGAGGGAATGGATACGATTGAGGGGTG | | |
| | | AGGGGCGCGGGGATGGGGCGA | | |
| AMP0059536 | Iriomote 8 | GAGGACAGCGACCAGAGCTGGAGGAGGATC | SEQ ID NO: | — |
| | | GATCGGCGGAGGGTGGTCTGTTATGTTCCGT | 315 | |
| | | GACCAGGCGTGACTCCGTGTCGCATATGATT | | |
| | | AGGGAAAGGATATGAACCGTGAGCTCCGTGG | | |
| | | CGCTGAAGTTGCGCCGGTTAGCTGGTCACTG | | |
| | | A | | |
| AMP0091501 | Iriomote 8 | TAACCCACCAAAGATAGCCTTGCATGCTCCTT | SEQ ID NO: | Present |
| | | AGTGTCGCTTATTTTGGTTTAAGACGGGTAAG | 316 | (AMP0111891) |
| | | TCTAGCTGAGTACCTTCTCGTACTCAGGGCGT | | |
| | | TGTTCTCATTGTTGTTGCAGATGGTTAGATGT | | |
| | | ACTATGGATATTGCGTCA | | |
| AMP0101156 | Iriomote 8 | TAGCCCACTAAAATAAAACCTTGCATACTCCT | SEQ ID NO: | — |
| | | TGGTGTCACTTTATTTCTGTTTAAGGCGGGTA | 317 | |
| | | AGTCTAGCTGAGTACCCTCTCGTACTCAGGG | | |
| | | CTTTGTTCCTATTGTTGTTGCAGATGGCCAGA | | |
| | | TGTACTATGGTTATTGCATCA | | |
| AMP0107476 | Iriomote 8 | TCGGAACGTCAAGGCCAAGTACATGAGCTCT | SEQ ID NO: | — |
| | | GGAGCACTCAGAAGCCAGACATCGATCAACT | 318 | |
| | | CATGTTGAGCCATCAGCGAGCCGTGTCGATC | | |
| | | CATCAAACAAGCCGTCCGA | | |

As shown in Table 16, the adjacent region comprising AMP0121265 located at 0 cM among the markers associated with sugarcane smut resistance derived from JW90 was found to comprise 36 markers. As shown in Table 17, the adjacent region comprising AMP0091501 located at 83.76 cM among the markers associated with sugarcane smut resistance derived from Iriomote 8 was found to comprise 18 markers. Among them, 5 markers were found to comprise the identical nucleotide sequence. On the basis of such results, the region comprising the 36 markers shown in Table 16 and the region comprising the 18 markers shown in Table 17 can be regarded as QTLs that are highly correlated with sugarcane smut resistance. In particular, both JW90 and Iriomote 8 have the QTL of sugarcane smut resistance identified in this example. Accordingly, such QTL is considered to be present in an extensive range of sugarcane varieties without particular limitation.

In particular, this example demonstrates that the 36 markers shown in Table 16 and the 18 markers shown in Table 17 can be used as particularly excellent markers associated with sugarcane smut resistance.

Among the 36 markers shown in Table 16 and the 18 markers shown in Table 17, in addition, 5 markers that were found to have the identical nucleotide sequence (i.e., AMP0016471 (SEQ ID NO: 298)=AMP0020554 (SEQ ID NO: 248), AMP0036426 (SEQ ID NO: 303)=AMP0045000 (SEQ ID NO: 270), AMP0046626 (SEQ ID NO: 307)=AMP0057239 (SEQ ID NO: 271), AMP0052709 (SEQ ID NO: 311)=AMP0062853 (SEQ ID NO: 273), and AMP0091501 (SEQ ID NO: 316)=AMP0111891 (SEQ ID NO: 292)) were found to be usable as the most excellent markers associated with sugarcane smut resistance.

All publications, patents, and patent applications cited herein are incorporated herein by reference in their entirety.

1. A method for producing a sugarcane line having improved smut resistance, the method comprising:
a step of breeding parent plants to obtain a progeny plant, wherein at least one of said parent plants is a sugarcane plant;
a step of extracting a genomic DNA of the progeny plant;
a step of determining the presence or absence of a marker associated with sugarcane smut resistance in the obtained genomic DNA;
a step of selecting a progeny plant having the marker associated with sugarcane smut resistance, and
a step of breeding only the selected progeny plant;
wherein the marker associated with sugarcane smut resistance comprises any nucleotide sequence selected from the group consisting of the nucleotide sequences of SEQ ID NOs: 144 to 151, or at least 15 consecutive nucleotides of SEQ ID NOs: 144 to 151.

2. The method for producing a sugarcane line according to claim 1, wherein the step of determination involves the use of a DNA chip comprising a probe corresponding to the marker associated with sugarcane smut resistance.

3. The method for producing a sugarcane line according to claim 1, wherein the progeny plant is a seed or young seedling and the genomic DNA is extracted from the seed or young seedling.

4. The method for producing a sugarcane line according to claim 1, wherein the nucleic acid region comprises the nucleotide sequence of SEQ ID NO:
1 or 2, or at least 15 consecutive nucleotides of SEQ ID NO: 149, 150 or 151.

5. The method for producing a sugarcane line according to claim 1, wherein the at least one of said parent plants is a smut-resistant strain of sugarcane.

6. The method for producing a sugarcane line according to claim 5, wherein the smut-resistant strain of sugarcane is selected from the group consisting of JW90, Iriomote 15, and Iriomote 8.
